SEQUENCE LISTING (171 sequences)

SEQ ID NO: 1 - protein, 248 aa, Artificial Sequence
  Synthetic Construct
QVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKR

SEQ ID NO: 2 - protein, 240 aa, Artificial Sequence
  Synthetic Construct
EVQLVESGGGLVQPKGSLKLSCAASGFTFNTYAMNWVRQAPGKGLEWVARIRNKSNNYATYYAASVKDRFTISRDDSQSMLYLQMNNLKTEDTAMYYCVSLGEFAYWGQGTLVTVSAGGGGSGGGGSGGGGSEIVLTQSPTTMAASPGEKVTITCSATSSINSNYLHWYQQRPGFSPKLLIYRTSNLASGVPARFSGSGSGTSYSLTIGTMEAEDVATYYCQQGSTLPFTFGSGTKLEIK

SEQ ID NO: 3 - protein, 240 aa, Artificial Sequence
  Synthetic Construct
EVQLVESGGGLVQPKGSLKLSCAASGFTFNTYAMNWVRQAPGKGLEWIARIRNKSNNYATYYAASVKDRFTISRDDSQSMLYLQMNNLKTEDTAMYYCVSLGEFAYWGQGTLVTVSAGGGGSGGGGSGGGGSEIVLTQSPTTMAASPGEKVTITCSATSSINSNYLHWYQQKPGFSPKLLIYRTSNLASGVPPRFSGSGSGTSYSLTIGTMEAEDVATYYCQQGSTLPFTFGSGTKLEIK

SEQ ID NO: 4 - protein, 246 aa, Artificial Sequence
  Synthetic Construct
EVQLVESGGGLVQPGRSLRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSGGGGSGGGGSGGGGSDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIK

SEQ ID NO: 5 - protein, 251 aa, Artificial Sequence
  Synthetic Construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSAGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKR

SEQ ID NO: 6 - protein, 234 aa, Artificial Sequence
  Synthetic Construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIK

SEQ ID NO: 7 - protein, 255 aa, Artificial Sequence
  Synthetic Construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSGGGGSGGGGSSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 8 - protein, 55 aa, Artificial Sequence
  Synthetic Construct
TMKHLWFFLLLVAAPRWVLSSWMEWDREINNYTSLIHSLIEESQNQQEKNEQELL

SEQ ID NO: 9 - protein, 5 aa, Artificial Sequence
  Synthetic Construct
GYGMH

SEQ ID NO: 10 - protein, 17 aa, Artificial Sequence
  Synthetic Construct
LISYDGSNKYYADSVKG

SEQ ID NO: 11 - protein, 16 aa, Artificial Sequence
  Synthetic Construct
GRNDFWSGYYTAGMDV

SEQ ID NO: 12 - protein, 11 aa, Artificial Sequence
  Synthetic Construct
QASQGIRKYLN

SEQ ID NO: 13 - protein, 7 aa, Artificial Sequence
  Synthetic Construct
DASNLET

SEQ ID NO: 14 - protein, 9 aa, Artificial Sequence
  Synthetic Construct
QQYDDFPFT

SEQ ID NO: 15 - protein, 5 aa, Artificial Sequence
  Synthetic Construct
TYAMN

SEQ ID NO: 16 - protein, 19 aa, Artificial Sequence
  Synthetic Construct
RIRNKSNNYATYYAASVKD

SEQ ID NO: 17 - protein, 6 aa, Artificial Sequence
  Synthetic Construct
LGEFAY

SEQ ID NO: 18 - protein, 12 aa, Artificial Sequence
  Synthetic Construct
SATSSINSNYLH

SEQ ID NO: 19 - protein, 7 aa, Artificial Sequence
  Synthetic Construct
RTSNLAS

SEQ ID NO: 20 - protein, 9 aa, Artificial Sequence
  Synthetic Construct
QQGSTLPFT

SEQ ID NO: 21 - protein, 5 aa, Artificial Sequence
  Synthetic Construct
NYGMA

SEQ ID NO: 22 - protein, 17 aa, Artificial Sequence
  Synthetic Construct
TISYDGSITYYRDSVKG

SEQ ID NO: 23 - protein, 11 aa, Artificial Sequence
  Synthetic Construct
EEQYSSWYFDF

SEQ ID NO: 24 - protein, 15 aa, Artificial Sequence
  Synthetic Construct
RASQSVSISSHDLMQ

SEQ ID NO: 25 - protein, 7 aa, Artificial Sequence
  Synthetic Construct
DAFNLAS

SEQ ID NO: 26 - protein, 9 aa, Artificial Sequence
  Synthetic Construct
QQSKDDPYT

SEQ ID NO: 27 - protein, 5 aa, Artificial Sequence
  Synthetic Construct
SYVIH

SEQ ID NO: 28 - protein, 17 aa, Artificial Sequence
  Synthetic Construct
YINPYNDGTDYDEKFKG

SEQ ID NO: 29 - protein, 13 aa, Artificial Sequence
  Synthetic Construct
EKDNYATGAWFAY

SEQ ID NO: 30 - protein, 17 aa, Artificial Sequence
  Synthetic Construct
KSSQSLLYSTNQKNYLA

SEQ ID NO: 31 - protein, 7 aa, Artificial Sequence
  Synthetic Construct
WASTRES

SEQ ID NO: 32 - protein, 8 aa, Artificial Sequence
  Synthetic Construct
QQYYSYRT

SEQ ID NO: 33 - protein, 5 aa, Artificial Sequence
  Synthetic Construct
GYYWS

SEQ ID NO: 34 - protein, 16 aa, Artificial Sequence
  Synthetic Construct
EINHSGSTNYNPSLKS

SEQ ID NO: 35 - protein, 7 aa, Artificial Sequence
  Synthetic Construct
VINWFDP

SEQ ID NO: 36 - protein, 11 aa, Artificial Sequence
  Synthetic Construct
RASQDISSWLA

SEQ ID NO: 37 - protein, 7 aa, Artificial Sequence
  Synthetic Construct
AASSLQS

SEQ ID NO: 38 - protein, 9 aa, Artificial Sequence
  Synthetic Construct
QQANSFPYT

SEQ ID NO: 39 - protein, 5 aa, Artificial Sequence
  Synthetic Construct
NYYWT

SEQ ID NO: 40 - protein, 16 aa, Artificial Sequence
  Synthetic Construct
YISDRESATYNPSLNS

SEQ ID NO: 41 - protein, 24 aa, Artificial Sequence
  Synthetic Construct
ARRGQRIYGVVSFGEFFYYYSMDV

SEQ ID NO: 42 - protein, 11 aa, Artificial Sequence
  Synthetic Construct
GRQALGSRAVQ

SEQ ID NO: 43 - protein, 7 aa, Artificial Sequence
  Synthetic Construct
NNQDRPS

SEQ ID NO: 44 - protein, 12 aa, Artificial Sequence
  Synthetic Construct
HMWDSRSGFSWS

SEQ ID NO: 45 - protein, 15 aa, Artificial Sequence
  Synthetic Construct
GGGGSGGGGSGGGGS

SEQ ID NO: 46 - protein, 5 aa, Artificial Sequence
  Synthetic Construct
  VARIANT (1)..(5): Can be present in repeats of any integer up to and including 8
GGGGS

SEQ ID NO: 47 - protein, 30 aa, Artificial Sequence
  Synthetic Construct
GGGGSGGGGSGGGGSGGGGSGGGGSGGGGS

SEQ ID NO: 48 - protein, 15 aa, Artificial Sequence
  Synthetic Construct
  VARIANT (1)..(15): Can be present in repeats of any integer up to and including 3
GSTSGSGKPGSGEGS

SEQ ID NO: 49 - protein, 1 aa, Artificial Sequence
  Synthetic Construct
  VARIANT 1: Can be present in repeats of any integer
G

SEQ ID NO: 50 - protein, 2 aa, Artificial Sequence
  Synthetic Construct
  VARIANT (1)..(2): Can be present in repeats of any integer
GS

SEQ ID NO: 51 - protein, 5 aa, Artificial Sequence
  Synthetic Construct
  VARIANT (1)..(5): Can be present in repeats of any integer up to and including 8
GSGGS

SEQ ID NO: 52 - protein, 4 aa, Artificial Sequence
  Synthetic Construct
  VARIANT (1)..(4): Can be present in repeats of any integer up to and including 10
GGGS

SEQ ID NO: 53 - protein, 15 aa, Artificial Sequence
  Synthetic Construct
EPKSSDKTHTSPPSP

SEQ ID NO: 54 - protein, 15 aa, Artificial Sequence
  Synthetic Construct
EPKSSDKGHGGPPGP

SEQ ID NO: 55 - protein, 12 aa, Artificial Sequence
  Synthetic Construct
ERKSSVESPPSP

SEQ ID NO: 56 - protein, 12 aa, Artificial Sequence
  Synthetic Construct
ERKSGVEGPPGP

SEQ ID NO: 57 - protein, 12 aa, Artificial Sequence
  Synthetic Construct
ESKYGPPSPPSP

SEQ ID NO: 58 - protein, 12 aa, Artificial Sequence
  Synthetic Construct
ESKYGPPGPPGP

SEQ ID NO: 59 - protein, 12 aa, Artificial Sequence
  Synthetic Construct
GGGGSGGGGSGS

SEQ ID NO: 60 - protein, 9 aa, Artificial Sequence
  Synthetic Construct
GGGGSGGGS

SEQ ID NO: 61 - protein, 254 aa, Artificial Sequence
  Synthetic Construct
EVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSSGGGGSGGGGSGGGGSSYELTQETGVSVALGRTVTITCRGDSLRSHYASWYQKKPGQAPILLFYGKNNRPSGVPDRFSGASGNRASLTISGAQAEDDAEYYCSSRDKSGSRLSVFGGGTKLTVL

SEQ ID NO: 62 - protein, 252 aa, Artificial Sequence
  Synthetic Construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 63 - protein, 5 aa, Artificial Sequence
  Synthetic Construct
NAWMT

SEQ ID NO: 64 - protein, 19 aa, Artificial Sequence
  Synthetic Construct
RITGPGEGWSVDYAAPVEG

SEQ ID NO: 65 - protein, 20 aa, Artificial Sequence
  Synthetic Construct
TGKYYDFWSGYPPGEEYFQD

SEQ ID NO: 66 - protein, 11 aa, Artificial Sequence
  Synthetic Construct
RGDSLRSHYAS

SEQ ID NO: 67 - protein, 7 aa, Artificial Sequence
  Synthetic Construct
GKNNRPS

SEQ ID NO: 68 - protein, 12 aa, Artificial Sequence
  Synthetic Construct
SSRDKSGSRLSV

SEQ ID NO: 69 - protein, 5 aa, Artificial Sequence
  Synthetic Construct
DSYWS

SEQ ID NO: 70 - protein, 16 aa, Artificial Sequence
  Synthetic Construct
YVHKSGDTNYSPSLKS

SEQ ID NO: 71 - protein, 24 aa, Artificial Sequence
  Synthetic Construct
TLHGRRIYGIVAFNEWFTYFYMDV

SEQ ID NO: 72 - protein, 11 aa, Artificial Sequence
  Synthetic Construct
GEKSLGSRAVQ

SEQ ID NO: 73 - protein, 7 aa, Artificial Sequence
  Synthetic Construct
NNQDRPS

SEQ ID NO: 74 - protein, 12 aa, Artificial Sequence
  Synthetic Construct
HIWDSRVPTKWV

SEQ ID NO: 75 - protein, 125 aa, Artificial Sequence
  Synthetic Construct
QVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSS

SEQ ID NO: 76 - protein, 108 aa, Artificial Sequence
  Synthetic Construct
DIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKR

SEQ ID NO: 77 - protein, 117 aa, Artificial Sequence
  Synthetic Construct
EVQLVESGGGLVQPKGSLKLSCAASGFTFNTYAMNWVRQAPGKGLEWVARIRNKSNNYATYYAASVKDRFTISRDDSQSMLYLQMNNLKTEDTAMYYCVSLGEFAYWGQGTLVTVSA

SEQ ID NO: 78 - protein, 108 aa, Artificial Sequence
  Synthetic Construct
EIVLTQSPTTMAASPGEKVTITCSATSSINSNYLHWYQQRPGFSPKLLIYRTSNLASGVPARFSGSGSGTSYSLTIGTMEAEDVATYYCQQGSTLPFTFGSGTKLEIK

SEQ ID NO: 79 - protein, 117 aa, Artificial Sequence
  Synthetic Construct
EVQLVESGGGLVQPKGSLKLSCAASGFTFNTYAMNWVRQAPGKGLEWIARIRNKSNNYATYYAASVKDRFTISRDDSQSMLYLQMNNLKTEDTAMYYCVSLGEFAYWGQGTLVTVSA

SEQ ID NO: 80 - protein, 108 aa, Artificial Sequence
  Synthetic Construct
EIVLTQSPTTMAASPGEKVTITCSATSSINSNYLHWYQQKPGFSPKLLIYRTSNLASGVPPRFSGSGSGTSYSLTIGTMEAEDVATYYCQQGSTLPFTFGSGTKLEIK

SEQ ID NO: 81 - protein, 120 aa, Artificial Sequence
  Synthetic Construct
EVQLVESGGGLVQPGRSLRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSS

SEQ ID NO: 82 - protein, 111 aa, Artificial Sequence
  Synthetic Construct
DIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIK

SEQ ID NO: 83 - protein, 122 aa, Artificial Sequence
  Synthetic Construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 84 - protein, 113 aa, Artificial Sequence
  Synthetic Construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKR

SEQ ID NO: 85 - protein, 112 aa, Artificial Sequence
  Synthetic Construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVT

SEQ ID NO: 86 - protein, 107 aa, Artificial Sequence [sequence omitted]

SEQ ID NO: 87 - protein, 132 aa, Artificial Sequence
  Synthetic Construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 88 - protein, 108 aa, Artificial Sequence
  Synthetic Construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 89 - protein, 131 aa, Artificial Sequence
  Synthetic Construct
EVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSS

SEQ ID NO: 90 - protein, 108 aa, Artificial Sequence
  Synthetic Construct
SYELTQETGVSVALGRTVTITCRGDSLRSHYASWYQKKPGQAPILLFYGKNNRPSGVPDRFSGASGNRASLTISGAQAEDDAEYYCSSRDKSGSRLSVFGGGTKLTVL

SEQ ID NO: 91 - protein, 129 aa, Artificial Sequence
  Synthetic Construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVT

SEQ ID NO: 92 - protein, 105 aa, Artificial Sequence
  Synthetic Construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 93 - protein, 7 aa, Artificial Sequence
  Synthetic Construct
TSGVSVG

SEQ ID NO: 94 - protein, 16 aa, Artificial Sequence
  Synthetic Construct
SINWNDDKCYSPSLKS

SEQ ID NO: 95 - protein, 17 aa, Artificial Sequence
  Synthetic Construct
DMPPHDSGPQSFDASDV

SEQ ID NO: 96 - protein, 11 aa, Artificial Sequence
  Synthetic Construct
SGDNLGDKYAC

SEQ ID NO: 97 - protein, 7 aa, Artificial Sequence
  Synthetic Construct
GDNKRPS

SEQ ID NO: 98 - protein, 9 aa, Artificial Sequence
  Synthetic Construct
QAWDTSTAV

SEQ ID NO: 99 - protein, 127 aa, Artificial Sequence
  Synthetic Construct
QVTLKESGPTLVKPTQTLTLTCTLSGFSLSTSGVSVGWIRQPPGKALEWLASINWNDDKCYSPSLKSRLTITKDTPKNQVVLAMSNMDPADTATYSCALDMPPHDSGPQSFDASDVWGPGTMVTVSS

SEQ ID NO: 100 - protein, 106 aa, Artificial Sequence
  Synthetic Construct
SYELMQLPSVSVSPGQTASITCSGDNLGDKYACWYQQKPGRSPVLVIYGDNKRPSGIPERFSGSNSGNTATLTISGTQAMDEADYYCQAWDTSTAVFGTGTKLTVL

SEQ ID NO: 101 - protein, 5 aa, Artificial Sequence
  Synthetic Construct
HDYWS

SEQ ID NO: 102 - protein, 16 aa, Artificial Sequence
  Synthetic Construct
FIFFDGSTNYNPSLNG

SEQ ID NO: 103 - protein, 20 aa, Artificial Sequence
  Synthetic Construct
LKGAWLLSEPPYFSSDGMDV

SEQ ID NO: 104 - protein, 13 aa, Artificial Sequence
  Synthetic Construct
SGSSSDIGSNTVN

SEQ ID NO: 105 - protein, 7 aa, Artificial Sequence
  Synthetic Construct
SNNQRPS

SEQ ID NO: 106 - protein, 11 aa, Artificial Sequence
  Synthetic Construct
AAWDESLNGVV

SEQ ID NO: 107 - protein, 128 aa, Artificial Sequence
  Synthetic Construct
QVQLQESGPGLVKPSETLSLTCTVSGGSIGHDYWSWIRQPPGEGLEWIGFIFFDGSTNYNPSLNGRVTISLDTSKNQLSLRLTSVTAADTAVYFCARLKGAWLLSEPPYFSSDGMDVWGQGTTVTVPS

SEQ ID NO: 108 - protein, 106 aa, Artificial Sequence
  Synthetic Construct
NFMLTQPPSASGTPGQRVSISCSGSSSDIGSNTVNWYQQLPGTAPKLLIYSNNQRPSGVPDRFSGFKSGTSASLVISGLQSEDEADYYCAAWDESLNGVVFGGGPR

SEQ ID NO: 109 - protein, 5 aa, Artificial Sequence
  Synthetic Construct
GYYWS

SEQ ID NO: 110 - protein, 16 aa, Artificial Sequence
  Synthetic Construct
EINHRGSTTYNPSLDG

SEQ ID NO: 111 - protein, 8 aa, Artificial Sequence
  Synthetic Construct
TVAGTSDY

SEQ ID NO: 112 - protein, 11 aa, Artificial Sequence
  Synthetic Construct
KASRDVDDDVN

SEQ ID NO: 113 - protein, 7 aa, Artificial Sequence
  Synthetic Construct
DATTLVP

SEQ ID NO: 114 - protein, 9 aa, Artificial Sequence
  Synthetic Construct
LQHDNFPLT

SEQ ID NO: 115 - protein, 116 aa, Artificial Sequence
  Synthetic Construct
QVQLQQWGAGLLKSWGTLSLTCAVSGASFSGYYWSWIRQPPGKGLEWIGEINHRGSTTYNPSLDGRVTISLDTSTNQISLKLTSMTAADTAVYYCARTVAGTSDYWGQGTLVTVSS

SEQ ID NO: 116 - protein, 107 aa, Artificial Sequence
  Synthetic Construct
KTTLTQSPAFMSATPGDKVSISCKASRDVDDDVNWYQQRPGEAPIFIIEDATTLVPGISPRFSGSGYGTDFTLTINNIDSEDAAYYFCLQHDNFPLTFGGGTKVEIK

SEQ ID NO: 117 - protein, 7 aa, Artificial Sequence
  Synthetic Construct
TTGEGVG

SEQ ID NO: 118 - protein, 16 aa, Artificial Sequence
  Synthetic Construct
LIYWDDDKRYSPSLKS

SEQ ID NO: 119 - protein, 16 aa, Artificial Sequence
  Synthetic Construct
EQYYYDTSGQPYYFDF

SEQ ID NO: 120 - protein, 11 aa, Artificial Sequence
  Synthetic Construct
RASQDIRKNLN

SEQ ID NO: 121 - protein, 7 aa, Artificial Sequence
  Synthetic Construct
DASDLET

SEQ ID NO: 122 - protein, 9 aa, Artificial Sequence
  Synthetic Construct
QQSDYLPLT

SEQ ID NO: 123 - protein, 126 aa, Artificial Sequence
  Synthetic Construct
QVTLKESGPTLVKPTQTLTLTCTFSGFSLRTTGEGVGWVRQPPGKALEWLALIYWDDDKRYSPSLKSRLTITKDTSKKQVVLTMTNVDPADTATYYCTHEQYYYDTSGQPYYFDFWGQGTLVTVSS

SEQ ID NO: 124 - protein, 107 aa, Artificial Sequence
  Synthetic Construct
NIQVTQSPSSLSASVGDRVTMTCRASQDIRKNLNWYQQKPGKAPKVLIYDASDLETGIPSRFSGSGSGTDFILTISSLQPEDIATYYCQQSDYLPLTFGGGTKVDIK

SEQ ID NO: 125 - protein, 5 aa, Artificial Sequence
  Synthetic Construct
NYWIG

SEQ ID NO: 126 - protein, 17 aa, Artificial Sequence
  Synthetic Construct
DIYPGGNYIRNNEKFKD

SEQ ID NO: 127 - protein, 13 aa, Artificial Sequence
  Synthetic Construct
SFGSNYVFAWFTY

SEQ ID NO: 128 - protein, 16 aa, Artificial Sequence
  Synthetic Construct
RSSQRLLSSYGHTYLH

SEQ ID NO: 129 - protein, 7 aa, Artificial Sequence
  Synthetic Construct
EVSNRFS

SEQ ID NO: 130 - protein, 9 aa, Artificial Sequence
  Synthetic Construct
SQSTHVPLT

SEQ ID NO: 131 - protein, 122 aa, Artificial Sequence
  Synthetic Construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 132 - protein, 112 aa, Artificial Sequence
  Synthetic Construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 133 - protein, 249 aa, Artificial Sequence
  Synthetic Construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSGGGGSGGGGSGGGGSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 134 - protein, 249 aa, Artificial Sequence
  Synthetic Construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKGGGGSGGGGSGGGGSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 135 - protein, 248 aa, Artificial Sequence
  Synthetic Construct
DIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSS

SEQ ID NO: 136 - protein, 313 aa, Artificial Sequence
  Synthetic Construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSGGGGSGGGGSSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLPGTPLGDTTHTSGYPYDVPDYALEGSGTTSGTTRLLSGHTCFTLTGLLGTLVTMGLLT

SEQ ID NO: 137 - protein, 306 aa, Artificial Sequence
  Synthetic Construct
SYELMQLPSVSVSPGQTASITCSGDNLGDKYACWYQQKPGRSPVLVIYGDNKRPSGIPERFSGSNSGNTATLTISGTQAMDEADYYCQAWDTSTAVFGTGTKLTVLGGGGSGGGGSGGGGSQVTLKESGPTLVKPTQTLTLTCTLSGFSLSTSGVSVGWIRQPPGKALEWLASINWNDDKCYSPSLKSRLTITKDTPKNQVVLAMSNMDPADTATYSCALDMPPHDSGPQSFDASDVWGPGTMVTVSSPGTPLGDTTHTSGYPYDVPDYALEGSGTTSGTTRLLSGHTCFTLTGLLGTLVTMGLLT

SEQ ID NO: 138 - protein, 307 aa, Artificial Sequence
  Synthetic Construct
QVQLQESGPGLVKPSETLSLTCTVSGGSIGHDYWSWIRQPPGEGLEWIGFIFFDGSTNYNPSLNGRVTISLDTSKNQLSLRLTSVTAADTAVYFCARLKGAWLLSEPPYFSSDGMDVWGQGTTVTVPSGGGGSGGGGSGGGGSNFMLTQPPSASGTPGQRVSISCSGSSSDIGSNTVNWYQQLPGTAPKLLIYSNNQRPSGVPDRFSGFKSGTSASLVISGLQSEDEADYYCAAWDESLNGVVFGGGPRPGTPLGDTTHTSGYPYDVPDYALEGSGTTSGTTRLLSGHTCFTLTGLLGTLVTMGLLT

SEQ ID NO: 139 - protein, 307 aa, Artificial Sequence
  Synthetic Construct
NFMLTQPPSASGTPGQRVSISCSGSSSDIGSNTVNWYQQLPGTAPKLLIYSNNQRPSGVPDRFSGFKSGTSASLVISGLQSEDEADYYCAAWDESLNGVVFGGGPRGGGGSGGGGSGGGGSQVQLQESGPGLVKPSETLSLTCTVSGGSIGHDYWSWIRQPPGEGLEWIGFIFFDGSTNYNPSLNGRVTISLDTSKNQLSLRLTSVTAADTAVYFCARLKGAWLLSEPPYFSSDGMDVWGQGTTVTVPSPGTPLGDTTHTSGYPYDVPDYALEGSGTTSGTTRLLSGHTCFTLTGLLGTLVTMGLLT

SEQ ID NO: 140 - protein, 566 aa, Artificial Sequence
  Synthetic Construct
QVQLQQWGAGLLKSWGTLSLTCAVSGASFSGYYWSWIRQPPGKGLEWIGEINHRGSTTYNPSLDGRVTISLDTSTNQISLKLTSMTAADTAVYYCARTVAGTSDYWGQGTLVTVSSGSASAPTGGGGSGGGGSGGGGSKTTLTQSPAFMSATPGDKVSISCKASRDVDDDVNWYQQRPGEAPIFIIEDATTLVPGISPRFSGSGYGTDFTLTINNIDSEDAAYYFCLQHDNFPLTFGGGTKVEIKPGTPLGDTTHTSGYPYDVPDYALEGSGTTSGTTRLLSGHTCFTLTGLLGTLVTMGLLTGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSG

SEQ ID NO: 141 - protein, 303 aa, Artificial Sequence
  Synthetic Construct
KTTLTQSPAFMSATPGDKVSISCKASRDVDDDVNWYQQRPGEAPIFIIEDATTLVPGISPRFSGSGYGTDFTLTINNIDSEDAAYYFCLQHDNFPLTFGGGTKVEIKGGGGSGGGGSGGGGSQVQLQQWGAGLLKSWGTLSLTCAVSGASFSGYYWSWIRQPPGKGLEWIGEINHRGSTTYNPSLDGRVTISLDTSTNQISLKLTSMTAADTAVYYCARTVAGTSDYWGQGTLVTVSSGSASAPTPGTPLGDTTHTSGYPYDVPDYALEGSGTTSGTTRLLSGHTCFTLTGLLGTLVTMGLLT

SEQ ID NO: 142 - protein, 306 aa, Artificial Sequence
  Synthetic Construct
QVTLKESGPTLVKPTQTLTLTCTFSGFSLRTTGEGVGWVRQPPGKALEWLALIYWDDDKRYSPSLKSRLTITKDTSKKQVVLTMTNVDPADTATYYCTHEQYYYDTSGQPYYFDFWGQGTLVTVSSGGGGSGGGGSGGGGSNIQVTQSPSSLSASVGDRVTMTCRASQDIRKNLNWYQQKPGKAPKVLIYDASDLETGIPSRFSGSGSGTDFILTISSLQPEDIATYYCQQSDYLPLTFGGGTKVDIKPGTPLGDTTHTSGYPYDVPDYALEGSGTTSGTTRLLSGHTCFTLTGLLGTLVTMGLLT

SEQ ID NO: 143 - protein, 306 aa, Artificial Sequence
  Synthetic Construct
NIQVTQSPSSLSASVGDRVTMTCRASQDIRKNLNWYQQKPGKAPKVLIYDASDLETGIPSRFSGSGSGTDFILTISSLQPEDIATYYCQQSDYLPLTFGGGTKVDIKGGGGSGGGGSGGGGSQVTLKESGPTLVKPTQTLTLTCTFSGFSLRTTGEGVGWVRQPPGKALEWLALIYWDDDKRYSPSLKSRLTITKDTSKKQVVLTMTNVDPADTATYYCTHEQYYYDTSGQPYYFDFWGQGTLVTVSSPGTPLGDTTHTSGYPYDVPDYALEGSGTTSGTTRLLSGHTCFTLTGLLGTLVTMGLLT

SEQ ID NO: 144 - protein, 307 aa, Artificial Sequence
  Synthetic Construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSGGGGSGGGGSGGGGSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKPGTPLGDTTHTSGYPYDVPDYALEGSGTTSGTTRLLSGHTCFTLTGLLGTLVTMGLLT

SEQ ID NO: 145 - protein, 307 aa, Artificial Sequence
  Synthetic Construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKGGGGSGGGGSGGGGSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSPGTPLGDTTHTSGYPYDVPDYALEGSGTTSGTTRLLSGHTCFTLTGLLGTLVTMGLLT

SEQ ID NO: 146 - protein, 306 aa, Artificial Sequence
  Synthetic Construct
QVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRPGTPLGDTTHTSGYPYDVPDYALEGSGTTSGTTRLLSGHTCFTLTGLLGTLVTMGLLT

SEQ ID NO: 147 - protein, 306 aa, Artificial Sequence
  Synthetic Construct
DIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSPGTPLGDTTHTSGYPYDVPDYALEGSGTTSGTTRLLSGHTCFTLTGLLGTLVTMGLLT

SEQ ID NO: 148 - protein, 21 aa, Artificial Sequence
  Synthetic Construct
MALPVTALLLPLALLLHAARP

SEQ ID NO: 149 - protein, 34 aa, Artificial Sequence
  Synthetic Construct
GSGTTSGTTRLLSGHTCFTLTGLLGTLVTMGLLT

SEQ ID NO: 150 - protein, 13 aa, Artificial Sequence
  Synthetic Construct
PGTPLGDTTHTSG

SEQ ID NO: 151 - protein, 11 aa, Artificial Sequence
  Synthetic Construct
YPYDVPDYALE

SEQ ID NO: 152 - protein, 24 aa, Artificial Sequence
  Synthetic Construct
PGTPLGDTTHTSGYPYDVPDYALE

SEQ ID NO: 153 - protein, 263 aa, Artificial Sequence
  Synthetic Construct
GSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSG

SEQ ID NO: 154 - protein, 5 aa, Artificial Sequence
  Synthetic Construct
MYGFN

SEQ ID NO: 155 - protein, 17 aa, Artificial Sequence
  Synthetic Construct
GIIPIFGTSNYAQKFRG

SEQ ID NO: 156 - protein, 22 aa, Artificial Sequence
  Synthetic Construct
DFGPDWEDGDSYDGSGRGFFDF

SEQ ID NO: 157 - protein, 12 aa, Artificial Sequence
  Synthetic Construct
RASQSVSSGSLA

SEQ ID NO: 158 - protein, 7 aa, Artificial Sequence
  Synthetic Construct
GASTRAT

SEQ ID NO: 159 - protein, 9 aa, Artificial Sequence
  Synthetic Construct
QQYGTSPYT

SEQ ID NO: 160 - protein, 132 aa, Artificial Sequence
  Synthetic Construct
RVQLLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSS

SEQ ID NO: 161 - protein, 107 aa, Artificial Sequence
  Synthetic Construct
VLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKR

SEQ ID NO: 162 - protein, 255 aa, Artificial Sequence
  Synthetic Construct
RVQLLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSSAGGGGSGGGGSGGGGSVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKR

SEQ ID NO: 163 - protein, 334 aa, Artificial Sequence
  Synthetic Construct
TMKHLWFFLLLVAAPRWVLSSRVQLLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSSAGGGGSGGGGSGGGGSVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRPGTPLGDTTHTSGYPYDVPDYALEGSGTTSGTTRLLSGHTCFTLTGLLGTLVTMGLLT

SEQ ID NO: 164 - protein, 21 aa, Artificial Sequence
  Synthetic Construct
TMKHLWFFLLLVAAPRWVLSS

SEQ ID NO: 165 - DNA, 10 bp, Artificial Sequence
  Synthetic Construct
  misc_feature 3, 4, 5, 6, 7, 8: n = A or T
ccnnnnnngg

SEQ ID NO: 166 - protein, 69 aa, Artificial Sequence
  Synthetic Construct
TTTPAPRPPTPAPTIASQPLSLRPEACRPAAGGAVHTRGLDFACDIYIWAPLAGTCGVLLLSLVITLYC

SEQ ID NO: 167 - protein, 341 aa, Artificial Sequence
  Synthetic Construct
MALPVTALLLPLALLLHAARPQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSAGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTTTPAPRPPTPAPTIASQPLSLRPEACRPAAGGAVHTRGLDFACDIYIWAPLAGTCGVLLLSLVITLYC

SEQ ID NO: 168 - protein, 338 aa, Artificial Sequence
  Synthetic Construct
MALPVTALLLPLALLLHAARPQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYC

SEQ ID NO: 169 - protein, 324 aa, Artificial Sequence
  Synthetic Construct
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYC

SEQ ID NO: 170 - protein, 344 aa, Artificial Sequence
  Synthetic Construct
MALPVTALLLPLALLLHAARPEVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSSGGGGSGGGGSGGGGSSYELTQETGVSVALGRTVTITCRGDSLRSHYASWYQKKPGQAPILLFYGKNNRPSGVPDRFSGASGNRASLTISGAQAEDDAEYYCSSRDKSGSRLSVFGGGTKLTVLTTTPAPRPPTPAPTIASQPLSLRPEACRPAAGGAVHTRGLDFACDIYIWAPLAGTCGVLLLSLVITLYC

SEQ ID NO: 171 - protein, 145 aa, Artificial Sequence
  Synthetic Construct
MALPVTALLLPLALLLHAARPTMKHLWFFLLLVAAPRWVLSSWMEWDREINNYTSLIHSLIEESQNQQEKNEQELLTTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2024/0050474 A1
(43) Pub. Date: Feb. 15, 2024

(54) ENGINEERED CELLS AND USES THEREOF

(71) Applicants: NANJING LEGEND BIOTECH CO., LTD., Jiangning District (CN); NANJING GENSCRIPT BIOTECH CO., LTD., Nanjing (CN)

(72) Inventors: Ming Zeng, Irving, TX (US); Weiming Wang, Nanjing Jiangsu (CN)

(21) Appl. No.: 18/264,612

(22) PCT Filed: Feb. 8, 2022
(86) PCT No.: PCT/CN2022/075476

§ 371 Date: Aug. 8, 2023

(30) Foreign Application Priority Data
Feb. 9, 2021 (WO) PCT/CN2021/076390

(51) Int. Cl. A61K 35/17; C07K 16/10; A61P 31/18; A61K 39/00; C07K 16/28

(52) U.S. Cl. CPC A61K 35/17; C07K 16/1045; A61P 31/18; A61K 39/4611; A61K 39/4631; A61K 39/464838; C07K 16/2866; A61K 39/464429; A61K 2239/13; A61K 2239/21

(57) ABSTRACT

Provided are engineered cells (such as stem cells or T cells) that have a surface molecule comprising a membrane-tethered binding moiety that binds to a T cell surface antigen (such as CCR5, CD4 or CXCR4) or a HIV antigen, or a membrane tethered inhibitory moiety that inhibits the membrane fusion of HIV (such as C34). Also provided are methods of making and using these engineered cells.

### CROSS REFERENCE TO RELATED APPLICATIONS

This application claims priority benefit of International Patent Application No, PCT/CN2021/076390 filed on Feb. 9, 2021, the contents of which are incorporated herein by reference in their entirely.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422003240SEQLIST.TXT, date recorded: Feb. 8, 2022, size: 133 KB).

### FIELD OF THE INVENTION

The invention relates to engineered cells comprising a surface molecule useful for treating infectious diseases such as HIV.

### BACKGROUND OF THE INVENTION

The chemokine (C-C motif) receptor 5 (CCR5) serves as an HIV-1 co-receptor and is essential for cell infection with CCR5-tropic viruses. Loss of functional receptor protects against HIV infection. See Gupta et al. Nature volume 568, pages 244-248 (2019). CCR5 gene-edited hematopoietic stem cells and progenitor cell (HSPC) transplantation is a promising strategy for HIV remission. However, only a fraction of HSPCs can be edited ex vivo to provide protection against infection prior to autologous transplantation. It was projected that by a mathematical model that a threshold of 73%-90% protected HSPCs in the transplant is sufficient to overcome transplantation-dependent loss of Simian immunodeficiency virus (SHIV) immunity. See Cardozo et al. bioRxiv 629717. However, Xu et al showed that donor-derived, sorted HSPCs edited with the use of CRISPR-Cas9 only resulted in CCR5 insertion or deletion (indel) efficiency of 17.8%. See Xu et al. N Engl J Med. 2019 Sep. 26; 381(13):1240-1247

The disclosures of all publications, patents, patent applications and published patent applications referred to herein are hereby incorporated herein by reference in their entirety.

### BRIEF SUMMARY OF THE INVENTION

The present application in one aspect provides an engineered cell comprising a nucleic acid encoding a surface molecule comprising: a) a binding moiety, wherein the binding moiety i) specifically binds to a T cell surface antigen and prevents the binding of the T cell surface antigen to its cognitive ligand on HIV, or ii) specifically binds to a HIV antigen competitively with 10-1074, 10E8, or PGT121, or binds to the same epitope as that of 10-1074, 10E8, or PGT121 and prevents HIV from infecting the engineered cell; and b) a membrane domain that tethers the molecule to the membrane or facilitates the tethering of the surface molecule to the membrane. In some embodiments, the surface molecule does not comprise an intracellular signaling domain. In some embodiments, the T cell surface antigen is selected from the group consisting of CCR5, CD4, and CXCR4.

In some embodiments, according to any of the engineered cells described above, the T cell surface antigen is CCR5.

The present application in another aspect provides an engineered cell comprising a nucleic acid encoding a surface molecule comprising: a) a binding moiety that specifically binds to CCR5 and prevents binding of CCR5 to an HIV protein, and b) a membrane domain that tethers the molecule to the membrane or facilitates the tethering of the surface molecule to the membrane, and the surface molecule does not comprise an intracellular signaling domain. In some embodiments, the membrane domain is derived from, or is a transmembrane domain from CD4, CD8, CD28, 4-1BB, or PD-1.

In some embodiments according to any of the engineered cells described above, the binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816.

In some embodiments, the binding moiety comprises an antibody moiety comprising a heavy chain variable region (V_{H}) and a light chain variable region (V_{L}), the V_{H} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14. In some embodiments, the antibody moiety comprises a) the V_{H} comprising the amino acid sequence set forth in SEQ ID NO: 75 or a variant comprising an amino acid sequence having at least about 80% sequence identity, and b) the V_{L} comprising the amino acid sequence of SEQ ID NO: 76 or a variant comprising an amino acid sequence having at least about 80% sequence identity.

In some embodiments, the binding moiety comprises an antibody moiety comprising a heavy chain variable region (V_{H}) and a light chain variable region (V_{L}), the Vu comprises the HC-CDR1 comprising the amino acid sequence of SEQ ID NO: 125, the HC-CDR2 comprising the amino acid sequence of SEQ ID NO: 126, and the HC-CDR3 comprising the amino acid sequence of SEQ ID NO: 127, and the V_{L} comprises the LC-CDR1 comprising the amino acid sequence of SEQ ID NO: 128, the LC-CDR2 comprising the amino acid sequence of SEQ ID NO: 129, and the LC-CDR3 comprising the amino acid sequence of SEQ ID NO: 130. In some embodiments, the antibody moiety comprises a) the Vu comprising the amino acid sequence set forth in SEQ ID NO: 131 or a variant comprising an amino acid sequence having at least about 80% sequence identity, and b) the V_{L} comprising the amino acid sequence of SEQ ID NO: 132 or a variant comprising an amino acid sequence having at least about 80% sequence identity.

In some embodiments, the binding moiety comprises an antibody moiety comprising a heavy chain variable region (V_{H}) and a light chain variable region (V_{L}), the Vu comprises the HC-CDR1 comprising the amino acid sequence of SEQ ID NO: 15, the HC-CDR2 comprising the amino acid sequence of SEQ ID NO: 16, and the HC-CDR3 comprising the amino acid sequence of SEQ ID NO: 17, and the V_{L} comprises the LC-CDR1 comprising the amino acid sequence of SEQ ID NO: 18, the LC-CDR2 comprising the amino acid sequence of SEQ ID NO: 19, and the LC-CDR3 comprising the amino acid sequence of SEQ ID NO: 20. In some embodiments, the antibody moiety comprises a) the Vu comprising the amino acid sequence set forth in SEQ ID NO: 77 or 79, or a variant comprising an amino acid sequence having at least about 80% sequence identity, and b) the V_{L} comprising the amino acid sequence of SEQ ID NO: 78 or 80, or a variant comprising an amino acid sequence having at least about 80% sequence identity.

In some embodiments, the antibody moiety comprises an amino acid sequence set forth in any of SEQ ID NOs: 1-3 and 133-135, or a variant comprising an amino acid sequence having at least about 80% sequence identity.

In some embodiments, the T cell surface antigen is CD4. In some embodiments, the binding moiety specifically binds to CD4 competitively with C2-05, C2-11, or C2-13, or specifically binds to the same epitope as that of C2-05, C2-11, or C2-13.

In some embodiments, the binding moiety comprises an antibody moiety comprising a heavy chain variable region (V_{H}) and a light chain variable region (V_{L}), wherein the Vu comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38. In some embodiments, the antibody moiety comprises a) the Vu comprising the amino acid sequence set forth in SEQ ID NO: 85, or a variant comprising an amino acid sequence having at least about 80% sequence identity, and b) the V_{L} comprising the amino acid sequence of SEQ ID NO: 86, or a variant comprising an amino acid sequence having at least about 80% sequence identity.

In some embodiments, the binding moiety comprises an antibody moiety comprising a heavy chain variable region (V_{H}) and a light chain variable region (V_{L}), the Vu comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26. In some embodiments, the antibody moiety comprises a) the Vu comprising the amino acid sequence set forth in SEQ ID NO: 81, or a variant comprising an amino acid sequence having at least about 80% sequence identity, and b) the V_{L} comprising the amino acid sequence of SEQ ID NO: 82, or a variant comprising an amino acid sequence having at least about 80% sequence identity.

In some embodiments, the binding moiety comprises the amino acid sequences of any of SEQ ID NOs: 4-6, or a variant comprising an amino acid sequence having at least about 80% sequence identity.

In some embodiments, the surface molecule comprises a binding moiety that specifically binds to HIV competitively with 10-1074, 10E8, or PGT121 specifically binds to the same epitope as that of 10-1074, 10E8, or PGT121.

In some embodiments, the surface molecule comprises a binding moiety that specifically binds to HIV competitively with 10-1074, or specifically binds to the same epitope as that of 10-1074. In some embodiments, the binding moiety comprises an antibody moiety comprising a heavy chain variable region (V_{H}) and a light chain variable region (V_{L}), the Vu comprises the HC-CDR1 comprising the amino acid sequence of SEQ ID NO: 39, the HC-CDR2 comprising the amino acid sequence of SEQ ID NO: 40, and the HC-CDR3 comprising the amino acid sequence of SEQ ID NO: 41, and the V_{L} comprises the LC-CDR1 comprising the amino acid sequence of SEQ ID NO: 42, the LC-CDR2 comprising the amino acid sequence of SEQ ID NO: 43, and the LC-CDR3 comprising the amino acid sequence of SEQ ID NO: 44. In some embodiments, the antibody moiety comprises a) the Vu comprising the amino acid sequence set forth in SEQ ID NO: 87, or a variant comprising an amino acid sequence having at least about 80% sequence identity, and b) the V_{L} comprising the amino acid sequence of SEQ ID NO: 88, or a variant comprising an amino acid sequence having at least about 80% sequence identity.

In some embodiments, the surface molecule comprises a binding moiety that specifically binds to HIV competitively with 10E8, or specifically binds to the same epitope as that of 10E8. In some embodiments, the binding moiety comprises an antibody moiety comprising a heavy chain variable region (V_{H}) and a light chain variable region (V_{L}), the Vu comprises the HC-CDR1 comprising the amino acid sequence of SEQ ID NO: 63, the HC-CDR2 comprising the amino acid sequence of SEQ ID NO: 64, and the HC-CDR3 comprising the amino acid sequence of SEQ ID NO: 65, and the V_{L} comprises the LC-CDR1 comprising the amino acid sequence of SEQ ID NO: 66, the LC-CDR2 comprising the amino acid sequence of SEQ ID NO: 67, and the LC-CDR3 comprising the amino acid sequence of SEQ ID NO: 68. In some embodiments, the antibody moiety comprises a) the Vu comprising the amino acid sequence set forth in SEQ ID NO: 89, or a variant comprising an amino acid sequence having at least about 80% sequence identity, and b) the V_{L} comprising the amino acid sequence of SEQ ID NO: 90, or a variant comprising an amino acid sequence having at least about 80% sequence identity.

In some embodiments, the surface molecule comprises a binding moiety that specifically binds to HIV competitively with PGT121, or specifically binds to the same epitope as that of PGT121. In some embodiments, the binding moiety comprises an antibody moiety comprising a heavy chain variable region (V_{H}) and a light chain variable region (V_{L}), the Vu comprises the HC-CDR1 comprising the amino acid sequence of SEQ ID NO: 69, the HC-CDR2 comprising the amino acid sequence of SEQ ID NO: 70, and the HC-CDR3 comprising the amino acid sequence of SEQ ID NO: 71, and the V_{L} comprises the LC-CDR1 comprising the amino acid sequence of SEQ ID NO: 72, the LC-CDR2 comprising the amino acid sequence of SEQ ID NO: 73, and the LC-CDR3 comprising the amino acid sequence of SEQ ID NO: 74. In some embodiments, the antibody moiety comprises a) the Vu comprising the amino acid sequence set forth in SEQ ID NO: 91, or a variant comprising an amino acid sequence having at least about 80% sequence identity, and b) the V_{L} comprising the amino acid sequence of SEQ ID NO: 92, or a variant comprising an amino acid sequence having at least about 80% sequence identity.

In some embodiments, the binding moiety comprises the amino acid sequences of SEQ ID NO: 7, 61, or 62, or a variant comprising an amino acid sequence having at least about 80% sequence identity.

In some embodiments according to any of the engineered cells described above, the V_{H} and the V_{L} are fused via a linker. In some embodiments, the linker between the V_{H} and the V_{L} is a peptide linker. In some embodiments, the linker between the V_{H} and the V_{L} comprises the amino acid sequence of SEQ ID NO: 45.

In some embodiments according to any of the engineered cells described above, the binding moiety is a sdAb, a scFv, a Fab′, a (Fab′)₂, an Fv, or a peptide ligand. In some embodiments, the binding moiety is a scFv.

In some embodiments according to any of the engineered cells described above, the engineered cell is a stem cell.

In some embodiments according to any of the engineered cells described above, the engineered cell is an immune cell. In some embodiments, the engineered cell is a T cell or a Natural Killer cell. In some embodiments, the T cell is gamma delta T cell.

The present application in another aspect provides an engineered cell comprising a nucleic acid encoding a surface molecule comprising: a) an inhibitory moiety that inhibits membrane fusion of HIV, the inhibitory moiety comprises the amino acid sequence of C34 or a functional portion thereof, and b) a membrane domain that tethers the molecule to the membrane or facilitates the tethering of the surface molecule to the membrane, and the engineered cell is a stem cell.

In some embodiments according to any of the engineered cells described above, the membrane domain comprises a Glycosylphosphatidylinositol (GPI) attachment signal sequence. In some embodiments, the GPI attachment signal sequence comprises an amino acid sequence set forth in SEQ ID NO: 149.

In some embodiments according to any of the engineered cells described above, the binding moiety or the inhibitory moiety is fused to N-terminus of the membrane domain via a linker. In some embodiments, the linker between a) the binding moiety or inhibitory moiety and b) the membrane domain comprises a peptide linker. In some embodiments, the peptide linker is selected from the group consisting of SEQ ID NOs: 45-52, 150 and 152.

In some embodiments according to any of the engineered cells described above, the surface molecule comprises a second binding moiety that specifically binds to a second antigen. In some embodiments, the binding moiety and the second binding moiety are linked in tandem. In some embodiments, the surface molecule comprises a) an anti-CCR5 antibody moiety or anti-CD4 antibody moiety that specifically binds to CCR5 or CD4, and b) an anti-HIV antibody moiety that specifically binds to a HIV antigen or an inhibitory moiety that inhibits membrane fusion of HIV.

In some embodiments according to any of the engineered cells described above, engineered cell comprises a second surface molecule, the second surface molecule comprising a second binding moiety that specifically binds to a second antigen. In some embodiments, the engineered cell comprises a) an anti-CCR5 antibody moiety or anti-CD4 antibody moiety that specifically binds to CCR5 or CD4, and b) an anti-HIV surface molecule comprising an antibody moiety that specifically binds to an HIV antigen or an inhibitory moiety that inhibits membrane fusion of HIV.

In some embodiments according to any of the engineered cells described above, the surface molecule comprises an amino acid sequence set forth in any of SEQ ID NOs: 136, and 144-147.

In some embodiments according to any of the engineered cells described above, the surface molecule further comprises a signal peptide at the N-terminus of the molecule that promotes the tethering of the surface molecule to the membrane. In some embodiments, the signal peptide is a CD8a signal peptide.

In some embodiments according to any of the engineered cells described above, the engineered cell expresses a chimeric antigen receptor. In some embodiments, the chimeric antigen receptor comprises an anti-HIV antibody moiety.

The present application in another aspect provides a plurality of the engineered cells such as any of the engineered cells described above, wherein upon a) mixture with a plurality of cells not expressing the surface molecule and are susceptible to HIV infection and b) contact of the cellular composition with an HIV, the percentage of cells not infected with the HIV is higher (such as at least about 10% higher) than the percentage of engineered cells. In some embodiments, the number of the plurality of cells not comprising a nucleic acid expressing the surface molecule is between about 80% to about 120% of number of the plurality of engineered cells. In some embodiments, the plurality of cells not comprising a nucleic acid expressing the surface molecule express CCR5, CXCR4 and/or CD4 and are susceptible to HIV.

The present application in another aspect provides a surface molecule comprising: a) a binding moiety that specifically binds to a T cell surface antigen and prevents the binding of the T cell surface antigen to its cognitive ligand on HIV, a binding moiety that specifically binds to a HIV antigen competitively with an anti-HIV antibody, or binds to the same epitope as that of the anti-HIV antibody and prevents HIV from infecting the engineered cell, or an inhibitory moiety that inhibits membrane fusion of HIV; and b) a membrane domain that can tether the molecule to a cell membrane or facilitate the tethering of the surface molecule to the cell membrane after being expressed in a cell, wherein upon being expressed by a cell, the cell confers herd immunity against HIV. In some embodiments, the cell expresses CCR5, CD4 or CXCR4. In some embodiments, the cell is a TZM-b1 cell. In some embodiments, the cell does not express CCR5, CD4 or CXCR4.

The present application in another aspect provides an engineered cell expressing the surface molecule described above. In some embodiments, the cell is a stem cell (such as a hematopoietic stem cell (HSC)). In some embodiments, the cell is an immune cell. In some embodiments, the cell is a T cell.

The present application in another aspect provides a pharmaceutical composition comprising any of the engineered cell or the plurality of engineered cells described above.

The present application in another aspect provides a method of preparing any of the engineered cells described above, comprising introducing/transducing one or more nucleic acids encoding the surface molecule into a cell, thereby obtaining the engineered cell. In some embodiments, the method further comprises selecting the engineered cell based on its expression of the surface molecule.

The present application in another aspect provides a method of enriching any of the engineered cell or the plurality of engineered cells described above, comprises selecting the engineered cell or cells based upon the binding moiety or the inhibitory moiety. In some embodiments, the engineered cell or cells are stem cells. In some embodiments, the engineered cells are T cells (such as gamma delta T cells). In some embodiments, the engineered cells are NK cells.

The present application in another aspect provides a method of treating an individual infected with HIV, comprising administering to the individual an effective amount of the pharmaceutical composition described above. In some embodiments, the engineered cells are autologous to the individual. In some embodiments, the engineered cells are allogeneic to the individual. In some embodiments, at least about 5% of the T cells in the individual express the surface molecule after administration of the pharmaceutical composition. In some embodiments, at least about 20% of the T cells in the individual are resistant to HIV infection after administration of the pharmaceutical composition. In some embodiments, the method further comprises administering a second therapy.

### BRIEF DESCRIPTION OF THE DRAWINGS

FIG. 1 depicts the blocking effects of cells expressing Glycosylphosphatidylinositol (GPI)-anchored various antibodies against HIV pseudovirus.

FIG. 2A depicts the GFP expression of TZM-b1 cells transduced with GPI-scFv constructs. FIG. 2B depicts the blocking effects of TZM-b1 cells transduced with GPI-scFv constructs against AD8 pseudovirus.

FIG. 3 depicts blocking effects of TZM-b1 cells transduced with various GPI-scFv constructs and TZM-b1 cells not transduced with GPI-scFv constructs in mixture against HIV pseudovirus.

FIG. 4 depicts blocking effects of TZM-b1 cells transduced with various GPI-scFv and TZM-b1 cells not transduced with a GPI-scFv construct in mixture against AD8 pseudovirus.

FIG. 5 depicts blocking effects of TZM-b1 cells transduced with a GPI-anti-CCR5 scFv constructs and TZM-b1 cells not transduced with a GPI-scFv construct in mixture against HIV pseudovirus under different virus concentrations.

FIGS. 6A-6B depicts blocking effects of TZM-b1 cells transduced with an exemplary GPI-scFv construct and TZM-b1 cells not transduced with a GPI-scFv construct in mixture against HIV pseudovirus.

FIG. 7 depicts percentage of CAR positive primary CD4 T cells expressing a CAR-scFv construct (CAR-C1-13 or CAR-10E8) before and after enrichment.

FIG. 8 depicts blocking effects of primary CD4 T cells expressing a CAR-scFv construct (CAR-CD4-11, CAR-CD4-13, or CAR-CCR5-13) and primary CD4 T cells without a CAR-scFv construct in mixture against HIV pseudovirus.

FIG. 9 depicts blocking effects of a mixture of two different types of cells (see Table 4 for details) against HIV pseudovirus.

### DETAILED DESCRIPTION OF THE INVENTION

The present application provides engineered cells (such as stem cells, such as immune cells) comprising a nucleic acid encoding a surface molecule comprising: a) a binding moiety that i) specifically binds to a T cell surface antigen and prevents the binding of the T cell surface antigen to its cognitive ligand on HIV or ii) specifically binds to a HIV antigen and prevents the binding of the HIV antigen to the engineered cell, or b) an inhibitory moiety that inhibits membrane fusion of HIV, the surface molecule further comprises a membrane domain (e.g., a GPI attachment sequence) that tethers the binding moiety or inhibitory moiety to the cell membrane or facilitates the tethering of the molecule to the membrane. By expressing the described surface molecule, the engineered cell achieves resistance to HIV infection. In some embodiments, the binding moiety specifically binds to CCR5, CD4, or CXCR4. In some embodiments, the binding moiety binds to a HIV antigen competitively with 10-1074, 10E8, or PGT121, or binds to the same epitope as that of 10-1074, 10E8, or PGT121. In some embodiments, the inhibitory moiety comprises the amino acid sequence of C34 or a functional portion thereof. In some embodiments, the surface molecule does not comprise an intracellular domain.

Also provided herein are cellular compositions comprising a plurality of the engineered cell described herein, pharmaceutical compositions comprising the engineered cells, methods of preparing the engineered cells, methods of treating an individual infected with HIV by administering the engineered cells.

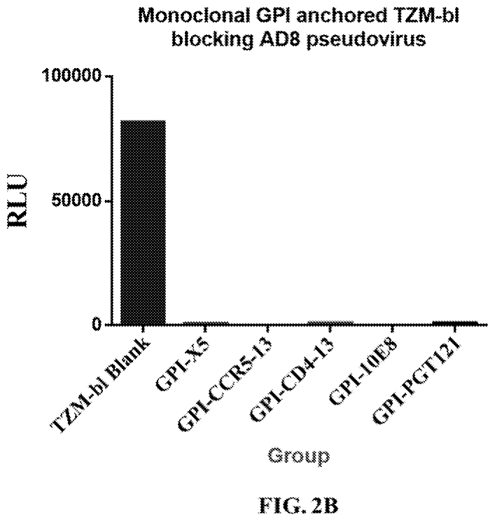

The present application in one aspect provides a solution to the long-lasting challenges in gene-editing based stem cell therapy for patients infected with HIV. For example, bi-allelic CCR5 knockout HSCs are HIV-resistant and can be a promising therapy. However, they are difficult to screen or enrich due to the lack of a selection marker. Selection marker cannot be integrated in ribonucleoprotein (RNP) under currently available genome-editing tools (such as CRISPR-Cas9). Moreover, high concentrations of RNP may lead to high off-target site cleavage. By expressing a surface molecule that confers HIV resistance on the engineered cells (such as stem cells, such as hematopoietic stem cells (HSCs)), a highly pure HIV-resistant cell population (e.g., a HSC population with more than 90% purity, see Example 2 and FIG. 2) can be obtained via enrichment based upon the surface molecule. This approach provides a feasible solution to the challenges for gene-editing based stem cell therapy, and great therapeutic benefits to treating HIV patients.

Moreover, by engineering the cells described in this application, we surprisingly found that engineered cells with certain surface molecules confer a cell-level herd immunity phenomenon. The engineered cells not only exhibit the resistance against HIV infection, but also confer immunity to other cells that are originally susceptible to HIV infection. Specifically, Examples 2-5 show that various engineered cells expressing exemplary surface molecules (such surface molecules that have a) an anti-CCR5, anti-CD4, or anti-HIV antibody moiety, or inhibitory moiety that inhibits membrane fusion of HIV and b) a membrane domain) confer herd immunity against HIV.

### Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term antibody includes conventional four-chain antibodies, and single-domain antibodies, such as heavy-chain only antibodies or fragments thereof, e.g., V_{H} H.

A full-length four-chain antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani, 1997, J. Mol. Biol., 273:927-948; Chothia 1985, J. Mol Biol., 186: 651-663; Chothia 1987, J. Mol. Biol., 196: 901-917; Chothia 1989, Nature, 342:877-883; Kabat 1987, Sequences of Proteins of Immunological Interest, Fourth Edition. US Govt. Printing Off. No. 165-492; Kabat 1991, Sequences of Proteins of Immunological Interest, Fifth Edition. NIH Publication No. 91-3242). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as 1gG1 (γ1 heavy chain), 1gG2 (γ2 heavy chain), 1gG3 (γ3 heavy chain), 1gG4 (γ4 heavy chain), 1gA1 (α1 heavy chain), or 1gA2 (α2 heavy chain).

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H} Hs” (Variable domain of the heavy chain of the Heavy chain antibody). Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H} H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The term “antibody moiety” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as 1gG1 (γ1 heavy chain), 1gG2 (γ2 heavy chain), 1gG3 (γ3 heavy chain), 1gG4 (γ4 heavy chain), 1gA1 (α1 heavy chain), or 1gA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains, which enables the scFv to form the desired structure for antigen binding. For a review of scFv, see Plûckthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs as defined by each of the above-cited references, are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein. Unless otherwise defined, the CDR sequences provided herein are based on Kabat definition.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

“Framework” or “FR” residues are those variable-domain residues other than the CDR residues as herein defined.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino acid sequence, which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R. Liss, 77 (1985); Boerner et al., J. Immunol. 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA 103:3557-3562 (2006) regarding human antibodies generated via a human B-cell hybridoma technology.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (MA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “specificity” refers to selective recognition of an antigen binding protein (such as a chimeric receptor or an antibody construct) for a particular epitope of an antigen. Natural antibodies, for example, are monospecific. The term “multispecific” as used herein denotes that an antigen binding protein has two or more antigen-binding sites of which at least two bind different antigens or epitopes. “Bispecific” as used herein denotes that an antigen binding protein has two different antigen-binding specificities. The term “monospecific” as used herein denotes an antigen binding protein that has one or more binding sites each of which bind the same antigen or epitope.

The term “valent” as used herein denotes the presence of a specified number of binding sites in an antigen binding protein. A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

“Chimeric antigen receptor” or “CAR” as used herein refers to genetically engineered receptors, which graft one or more antigen specificity onto cells, such as T cells. CARs are also known as “artificial T-cell receptors,” “chimeric T cell receptors,” or “chimeric immune receptors.” In some embodiments, the CAR comprises an extracellular variable domain of an antibody specific for a tumor antigen, and an intracellular signaling domain of a T cell receptor and/or other receptors, such as one or more costimulatory domains. “CAR-T” refers to a T cell that expresses a CAR.

“T cell receptor” or “TCR” as used herein refers to endogenous or recombinant T cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR specifically binds a tumor antigen. “TCR-T” refers to a T cell that expresses a recombinant TCR.

“Chimeric T cell receptor” or “cTCR” as used herein refers to an engineered receptor comprising an extracellular antigen-binding domain that binds to a specific antigen, a transmembrane domain of a first subunit of the TCR complex or a portion thereof, and an intracellular signaling domain of a second subunit of the TCR complex or a portion thereof, wherein the first or second subunit of the TCR complex is a TCRα chain, TCRβ chain, TCRγ chain, TCRδ chain, CD3ε, CD3δ, or CD3γ. The transmembrane domain and the intracellular signaling domain of a cTCR may be derived from the same subunit of the TCR complex, or from different subunits of the TCR complex. The intracellular domain may be the full-length intracellular signaling domain or a portion of the intracellular domain of a naturally occurring TCR subunit. In some embodiments, the cTCR comprises the extracellular domain of the TCR subunit or a portion thereof. In some embodiments, the cTCR does not comprise the extracellular domain of the TCR subunit. An “eTCR” refers to a cTCR comprising an extracellular domain of CD3c.

“Percent (%) amino acid sequence identity” with respect to a polypeptide sequence are defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full-length of the sequences being compared. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

The term “express” refers to translation of a nucleic acid into a protein. Proteins may be expressed and remain intracellular, become a component of the cell surface membrane, or be secreted into extracellular matrix or medium.

The term “host cell” refers to a cell that can support the replication or expression of the expression vector. Host cells may be prokaryotic cells such as E. coli, or eukaryotic cells, such as yeast, insect cells, amphibian cells, or mammalian cells.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid.

The term “in vivo” refers to inside the body of the organism from which the cell is obtained. “Ex vivo” or “in vitro” means outside the body of the organism from which the cell is obtained.

The term “cell” includes the primary subject cell and its progeny.

“Activation”, as used herein in relation to a cell expressing CD3, refers to the state of the cell that has been sufficiently stimulated to induce a detectable increase in downstream effector functions of the CD3 signaling pathway, including, without limitation, cellular proliferation and cytokine production.

As used herein, the term “autologous” refers to any material derived from the same individual to whom it is later to be re-introduced into the individual.

“Allogeneic” refers to a graft derived from a different individual of the same species.

The term “domain” when referring to a portion of a protein is meant to include structurally and/or functionally related portions of one or more polypeptides that make up the protein. For example, a transmembrane domain of an immune cell receptor may refer to the portions of each polypeptide chain of the receptor that span the membrane. A domain may also refer to related portions of a single polypeptide chain. For example, a transmembrane domain of a monomeric receptor may refer to portions of the single polypeptide chain of the receptor that span the membrane. A domain may also include only a single portion of a polypeptide.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “inducible promoter” refers to a promoter whose activity can be regulated by adding or removing one or more specific signals. For example, an inducible promoter may activate transcription of an operably linked nucleic acid under a specific set of conditions, e.g., in the presence of an inducing agent or conditions that activates the promoter and/or relieves repression of the promoter.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume in cancer). The methods of the invention contemplate any one or more of these aspects of treatment.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

Administration “in combination with” one or more further agents includes simultaneous and sequential administration in any order.

The term “simultaneously” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 15 minutes, such as no more than about any of 10, 5, or 1 minute.

The term “sequentially” is used herein to refer to administration of two or more therapeutic agents where the administration of one or more therapeutic agent(s) continues after discontinuing the administration of one or more other agent(s). For example, administration of the two or more agents are administered with a time separation of more than about 15 minutes, such as about any of 20, 30, 40, 50, or 60 minutes, 1 day, 2 days, 3 days, 1 week, 2 weeks, or 1 month, or longer.

A “subject” or an “individual” for purposes of treatment refers to any animal classified as a mammal, including humans, domestic and farm animals, and zoo, sports, or pet animals, such as dogs, horses, cats, cows, etc.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “an,” and “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### Engineered Cell Comprising a Nucleic Acid Encoding a Surface Molecule

The present application in one aspect provides an engineered cell comprising a nucleic acid encoding a surface molecule comprising a) a binding moiety that i) specifically binds to a T cell surface antigen and prevents the binding of the T cell surface antigen to its cognitive ligand on HIV, or ii) specifically binds to a HIV antigen, and b) a membrane domain that tethers the molecule to the membrane or facilitates the tethering of the molecule to the membrane. In some embodiments, the surface molecule does not comprise an intracellular signaling domain. In some embodiments, the T cell surface antigen is selected from the group consisting of CCR5, CD4, and CXCR4. In some embodiments, the membrane domain comprises a GPI attachment signal sequence (e.g., SEQ ID NO: 149).

In some embodiments, there is provided an engineered stem cell (e.g., hematopoietic stem cell (HSC)) comprising a) a binding moiety, wherein the binding moiety specifically binds to a T cell surface antigen and prevents the binding of the T cell surface antigen to its cognitive ligand on HIV, wherein the T cell surface antigen is selected from the group consisting of CCR5, CD4, and CXCR4, and b) a membrane domain that tethers the molecule to the membrane or facilitates the tethering of the molecule to the membrane. In some embodiments, the surface molecule does not comprise an intracellular signaling domain.

In some embodiments, there is provided an engineered stem cell (e.g., hematopoietic stem cell (HSC)) comprising a) a binding moiety, wherein the binding moiety specifically binds to CCR5, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., hematopoietic stem cell (HSC)) comprising a) a binding moiety, wherein the binding moiety specifically binds to CCR5, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14. In some embodiments, the antibody moiety comprises a) the VH comprising the amino acid sequence set forth in SEQ ID NO: 75 or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 76 or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequence of SEQ ID NO: 1, or a variant comprising an amino acid sequence having at least about 80% (such as at least about any one of 80%, 85%, 90%, 95%, 96%, 97%, 98%, or 99%) sequence identity. In some embodiments, the binding moiety comprises an antibody moiety comprising a heavy chain variable region (VH) and a light chain variable region (VL), wherein the VH comprises the HC-CDR1 comprising the amino acid sequence of SEQ ID NO: 125, the HC-CDR2 comprising the amino acid sequence of SEQ ID NO: 126, and the HC-CDR3 comprising the amino acid sequence of SEQ ID NO: 127, and the VL comprises the LC-CDR1 comprising the amino acid sequence of SEQ ID NO: 128, the LC-CDR2 comprising the amino acid sequence of SEQ ID NO: 129, and the LC-CDR3 comprising the amino acid sequence of SEQ ID NO: 130. In some embodiments, the antibody moiety comprises a) the VH comprising the amino acid sequence set forth in SEQ ID NO: 131 or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 132 or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequence of SEQ ID NO: 133 or 134, or a variant comprising an amino acid sequence having at least about 80% (such as at least about any one of 80%, 85%, 90%, 95%, 96%, 97%, 98%, or 99%) sequence identity. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 15, the HC-CDR2 comprising the amino acid sequence of SEQ ID NO: 16, and the HC-CDR3 comprising the amino acid sequence of SEQ ID NO: 17, and the V_{L} comprises the LC-CDR1 comprising the amino acid sequence of SEQ ID NO: 18, the LC-CDR2 comprising the amino acid sequence of SEQ ID NO: 19, and the LC-CDR3 comprising the amino acid sequence of SEQ ID NO: 20. In some embodiments, the antibody moiety comprises a) the VH comprising the amino acid sequence set forth in SEQ ID NO: 77 or 79, or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 78 or 80, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequence of SEQ ID NO: 2 or 3, or a variant comprising an amino acid sequence having at least about 80% (such as at least about any one of 80%, 85%, 90%, 95%, 96%, 97%, 98%, or 99%) sequence identity.

In some embodiments, there is provided an engineered stem cell (e.g., hematopoietic stem cell (HSC)) comprising a) a binding moiety comprising an scFv that specifically binds to CCR5, wherein the scFv comprises the anyone of the amino acid sequence of SEQ ID NO: 1-3 and 133-135, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety comprising an scFv that specifically binds to CCR5, wherein the scFv comprises the amino acid sequence of SEQ ID NO: 1-3 and 133-135, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered stem cell (e.g., a hematopoietic stem cell (a HSC)) comprising a) a binding moiety, wherein the binding moiety specifically binds to CD4, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety, wherein the binding moiety specifically binds to CD4, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety specifically binds to CD4 competitively with C2-05, C2-11, or C2-13, or specifically binds to the same epitope as that of C2-05, C2-11, or C2-13. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38. In some embodiments, the antibody moiety comprises a) the VH comprising the amino acid sequence set forth in SEQ ID NO: 85, or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 86, or a variant comprising an amino acid sequence having at least about 80% (such as at least about any one of 80%, 85%, 90%, 95%, 96%, 97%, 98%, or 99%) sequence identity. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26. In some embodiments, the antibody moiety comprises a) the VH comprising the amino acid sequence set forth in SEQ ID NO: 81, or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 82, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequences of any of SEQ ID NOs: 4-6, or a variant comprising an amino acid sequence having at least about 80% (such as at least about any one of 80%, 85%, 90%, 95%, 96%, 97%, 98%, or 99%) sequence identity.

In some embodiments, there is provided an engineered stem cell (e.g., a hematopoietic stem cell (a HSC)) comprising a) a binding moiety comprising an scFv that specifically binds to CD4, wherein the scFv comprises the amino acid sequence of any of SEQ ID NOs: 4-6, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety comprising an scFv that specifically binds to CD4, wherein the scFv comprises the amino acid sequence of any of SEQ ID NOs: 4-6, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered stem cell (e.g., a hematopoietic stem cell (a HSC)) comprising a) a binding moiety, wherein the binding moiety specifically binds to a HIV antigen competitively with 10-1074, 10E8, or PGT121, or binds to the same epitope as that of 10-1074, 10E8, or PGT121 and prevents the binding of the HIV antigen to the engineered cell, and b) a membrane domain that tethers the molecule to the membrane or facilitates the tethering of the molecule to the membrane. In some embodiments, the surface molecule does not comprise an intracellular signaling domain.

In some embodiments, there is provided an engineered stem cell (e.g., a hematopoietic stem cell (a HSC)) comprising a) a binding moiety, wherein the binding moiety specifically binds to HIV competitively with 10-1074, or specifically binds to the same epitope as that of 10-1074, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety, wherein the binding moiety specifically binds to HIV competitively with 10-1074, or specifically binds to the same epitope as that of 10-1074, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 39, the HC-CDR2 comprising the amino acid sequence of SEQ ID NO: 40, and the HC-CDR3 comprising the amino acid sequence of SEQ ID NO: 41, and the V_{L} comprises the LC-CDR1 comprising the amino acid sequence of SEQ ID NO: 42, the LC-CDR2 comprising the amino acid sequence of SEQ ID NO: 43, and the LC-CDR3 comprising the amino acid sequence of SEQ ID NO: 44. In some embodiments, the antibody moiety comprises a) the VH comprising the amino acid sequence set forth in SEQ ID NO: 87, or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 88, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequences of SEQ ID NO: 7, or a variant comprising an amino acid sequence having at least about 80% (such as at least about any one of 80%, 85%, 90%, 95%, 96%, 97%, 98%, or 99%) sequence identity.

In some embodiments, there is provided an engineered stem cell (e.g., a hematopoietic stem cell (a HSC)) comprising a) a binding moiety, wherein the binding moiety specifically binds to HIV competitively with 10E8, or specifically binds to the same epitope as that of 10E8, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety, wherein the binding moiety specifically binds to HIV competitively with 10E8, or specifically binds to the same epitope as that of 10E8, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 63, the HC-CDR2 comprising the amino acid sequence of SEQ ID NO: 64, and the HC-CDR3 comprising the amino acid sequence of SEQ ID NO: 65, and the V_{L} comprises the LC-CDR1 comprising the amino acid sequence of SEQ ID NO: 66, the LC-CDR2 comprising the amino acid sequence of SEQ ID NO: 67, and the LC-CDR3 comprising the amino acid sequence of SEQ ID NO: 68. In some embodiments, the antibody moiety comprises a) the VH comprising the amino acid sequence set forth in SEQ ID NO: 89, or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 90, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequences of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, there is provided an engineered stem cell (e.g., a hematopoietic stem cell (a HSC)) comprising a) a binding moiety comprising a scFv comprising the amino acid sequences of SEQ ID NO: 61, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety comprising a scFv comprising the amino acid sequences of SEQ ID NO: 61, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered stem cell (e.g., a hematopoietic stem cell (a HSC)) comprising a) a binding moiety, wherein the binding moiety specifically binds to HIV competitively with PGT121, or specifically binds to the same epitope as that of PGT121, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety, wherein the binding moiety specifically binds to HIV competitively with PGT121, or specifically binds to the same epitope as that of PGT121, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 69, the HC-CDR2 comprising the amino acid sequence of SEQ ID NO: 70, and the HC-CDR3 comprising the amino acid sequence of SEQ ID NO: 71, and the V_{L} comprises the LC-CDR1 comprising the amino acid sequence of SEQ ID NO: 72, the LC-CDR2 comprising the amino acid sequence of SEQ ID NO: 73, and the LC-CDR3 comprising the amino acid sequence of SEQ ID NO: 74. In some embodiments, the antibody moiety comprises a) the VH comprising the amino acid sequence set forth in SEQ ID NO: 91, or a variant comprising an amino acid sequence having at least about 80% (such as at least about any one of 80%, 85%, 90%, 95%, 96%, 97%, 98%, or 99%) sequence identity, and b) the VL comprising the amino acid sequence of SEQ ID NO: 92, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequences of SEQ ID NO: 62, or a variant comprising an amino acid sequence having at least about 80% (such as at least about any one of 80%, 85%, 90%, 95%, 96%, 97%, 98%, or 99%) sequence identity.

In some embodiments, there is provided an engineered stem cell (e.g., a hematopoietic stem cell (a HSC)) comprising a) a binding moiety comprising a scFv comprising the amino acid sequences of SEQ ID NO: 62, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) a binding moiety comprising a scFv comprising the amino acid sequences of SEQ ID NO: 62, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered stem cell (e.g., a hematopoietic stem cell (a HSC)) comprising a) an inhibitory moiety that inhibits membrane fusion of HIV, and b) a membrane domain that tethers the molecule to the membrane or facilitates the tethering of the molecule to the membrane. In some embodiments, the inhibitory moiety is selected from the group consisting of C34, HP32, SC35EK, sifuvirtide, T20, and T2634, or functional portions thereof. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, there is provided an engineered stem cell (e.g., a hematopoietic stem cell (a HSC)) comprising a) an inhibitory moiety comprising the amino acid sequence of C34 (SEQ ID NO: 8) or a function portion thereof, and b) a membrane domain comprising a GPI attachment signal sequence (e.g., SEQ ID NO: 149), wherein the surface molecule does not comprise an intracellular signaling domain. In some embodiments, there is provided an engineered stem cell (e.g., a hematopoietic stem cell (a HSC)) comprising a) an inhibitory moiety comprising the amino acid sequence of C34 (SEQ ID NO: 8) or a function portion thereof, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety, wherein the binding moiety specifically binds to a T cell surface antigen and prevents the binding of the T cell surface antigen to its cognitive ligand on HIV, wherein the T cell surface antigen is selected from the group consisting of CCR5, CD4, and CXCR4, and b) a membrane domain that tethers the molecule to the membrane or facilitates the tethering of the molecule to the membrane. In some embodiments, the surface molecule does not comprise an intracellular signaling domain.

In some embodiments, there is provided an engineered immune cell (e.g., a T cell) comprising a) a binding moiety, wherein the binding moiety specifically binds to CCR5, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety, wherein the binding moiety specifically binds to CCR5, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14. In some embodiments, the binding moiety comprises the amino acid sequence of SEQ ID NO: 1, or a variant comprising an amino acid sequence having at least about 80% (such as at least about any one of 80%, 85%, 90%, 95%, 96%, 97%, 98%, or 99%) sequence identity. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 15, the HC-CDR2 comprising the amino acid sequence of SEQ ID NO: 16, and the HC-CDR3 comprising the amino acid sequence of SEQ ID NO: 17, and the V_{L} comprises the LC-CDR1 comprising the amino acid sequence of SEQ ID NO: 18, the LC-CDR2 comprising the amino acid sequence of SEQ ID NO: 19, and the LC-CDR3 comprising the amino acid sequence of SEQ ID NO: 20. In some embodiments, the binding moiety comprises the amino acid sequence of SEQ ID NO: 2, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequence of SEQ ID NO: 3, or a variant comprising an amino acid sequence having at least about 80% (such as at least about any one of 80%, 85%, 90%, 95%, 96%, 97%, 98%, or 99%) sequence identity.

In some embodiments, there is provided an engineered immune cell (e.g., a T cell) comprising a) a binding moiety comprising an scFv that specifically binds to CCR5, wherein the scFv comprises the amino acid sequence of SEQ ID NO: 1, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising an scFv that specifically binds to CCR5, wherein the scFv comprises the amino acid sequence of SEQ ID NO: 1, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety comprising an scFv that specifically binds to CCR5, wherein the scFv comprises the amino acid sequence of SEQ ID NO: 2, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising an scFv that specifically binds to CCR5, wherein the scFv comprises the amino acid sequence of SEQ ID NO: 2, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety comprising an scFv that specifically binds to CCR5, wherein the scFv comprises the amino acid sequence of SEQ ID NO: 3, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising an scFv that specifically binds to CCR5, wherein the scFv comprises the amino acid sequence of SEQ ID NO: 3, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety, wherein the binding moiety specifically binds to CD4, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety, wherein the binding moiety specifically binds to CD4, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety specifically binds to CD4 competitively with C2-05, C2-11, or C2-13, or specifically binds to the same epitope as that of C2-05, C2-11, or C2-13. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38. In some embodiments, the binding moiety comprises the amino acid sequences of SEQ ID NO: 6, or a variant comprising an amino acid sequence having at least about 80% (such as at least about any one of 80%, 85%, 90%, 95%, 96%, 97%, 98%, or 99%) sequence identity. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26. In some embodiments, the binding moiety comprises the amino acid sequences of SEQ ID NO: 4, or a variant comprising an amino acid sequence having at least about 80% (such as at least about any one of 80%, 85%, 90%, 95%, 96%, 97%, 98%, or 99%) sequence identity.

In some embodiments, there is provided an engineered immune cell (e.g., a T cell) comprising a) a binding moiety comprising an scFv that specifically binds to CD4, wherein the scFv comprises the amino acid sequence of SEQ ID NO: 4, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising an scFv that specifically binds to CD4, wherein the scFv comprises the amino acid sequence of SEQ ID NO: 4, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety comprising an scFv that specifically binds to CD4, wherein the scFv comprises the amino acid sequence of SEQ ID NO: 6, or a variant comprising an amino acid sequence having at least about 80% (such as at least about any one of 80%, 85%, 90%, 95%, 96%, 97%, 98%, or 99%) sequence identity, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising an scFv that specifically binds to CD6, wherein the scFv comprises the amino acid sequence of SEQ ID NO: 4, or a variant comprising an amino acid sequence having at least about 80% (such as at least about any one of 80%, 85%, 90%, 95%, 96%, 97%, 98%, or 99%) sequence identity, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety, wherein the binding moiety specifically binds to a HIV antigen competitively with 10-1074, 10E8, or PGT121, or binds to the same epitope as that of 10-1074, 10E8, or PGT121 and prevents the binding of the HIV antigen to the engineered cell, and b) a membrane domain that tethers the molecule to the membrane or facilitates the tethering of the molecule to the membrane. In some embodiments, the surface molecule does not comprise an intracellular signaling domain.

In some embodiments, there is provided an engineered immune cell (e.g., a T cell) comprising a) a binding moiety, wherein the binding moiety specifically binds to HIV competitively with 10-1074, or specifically binds to the same epitope as that of 10-1074, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety, wherein the binding moiety specifically binds to HIV competitively with 10-1074, or specifically binds to the same epitope as that of 10-1074, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 39, the HC-CDR2 comprising the amino acid sequence of SEQ ID NO: 40, and the HC-CDR3 comprising the amino acid sequence of SEQ ID NO: 41, and the V_{L} comprises the LC-CDR1 comprising the amino acid sequence of SEQ ID NO: 42, the LC-CDR2 comprising the amino acid sequence of SEQ ID NO: 43, and the LC-CDR3 comprising the amino acid sequence of SEQ ID NO: 44. In some embodiments, the binding moiety comprises the amino acid sequences of SEQ ID NO: 7, or a variant comprising an amino acid sequence having at least about 80% sequence identity.

In some embodiments, there is provided an engineered immune cell (e.g., a T cell) comprising a) a binding moiety, wherein the binding moiety specifically binds to HIV competitively with 10E8, or specifically binds to the same epitope as that of 10E8, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety, wherein the binding moiety specifically binds to HIV competitively with 10E8, or specifically binds to the same epitope as that of 10E8, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 63, the HC-CDR2 comprising the amino acid sequence of SEQ ID NO: 64, and the HC-CDR3 comprising the amino acid sequence of SEQ ID NO: 65, and the V_{L} comprises the LC-CDR1 comprising the amino acid sequence of SEQ ID NO: 66, the LC-CDR2 comprising the amino acid sequence of SEQ ID NO: 67, and the LC-CDR3 comprising the amino acid sequence of SEQ ID NO: 68. In some embodiments, the binding moiety comprises the amino acid sequences of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, there is provided an engineered immune cell (e.g., a T cell) comprising a) a binding moiety comprising a scFv comprising the amino acid sequences of SEQ ID NO: 61, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising a scFv comprising the amino acid sequences of SEQ ID NO: 61, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) a binding moiety, wherein the binding moiety specifically binds to HIV competitively with PGT121, or specifically binds to the same epitope as that of PGT121, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety, wherein the binding moiety specifically binds to HIV competitively with PGT121, or specifically binds to the same epitope as that of PGT121, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 69, the HC-CDR2 comprising the amino acid sequence of SEQ ID NO: 70, and the HC-CDR3 comprising the amino acid sequence of SEQ ID NO: 71, and the V_{L} comprises the LC-CDR1 comprising the amino acid sequence of SEQ ID NO: 72, the LC-CDR2 comprising the amino acid sequence of SEQ ID NO: 73, and the LC-CDR3 comprising the amino acid sequence of SEQ ID NO: 74. In some embodiments, the binding moiety comprises the amino acid sequences of SEQ ID NO: 62, or a variant comprising an amino acid sequence having at least about 80% (such as at least about any one of 80%, 85%, 90%, 95%, 96%, 97%, 98%, or 99%) sequence identity.

In some embodiments, there is provided an engineered immune cell (e.g., a T cell) comprising a) a binding moiety comprising a scFv comprising the amino acid sequences of SEQ ID NO: 62, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) a binding moiety comprising a scFv comprising the amino acid sequences of SEQ ID NO: 62, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, there is provided an engineered immune cell (e.g., a T cell) comprising a) an inhibitory moiety that inhibits membrane fusion of HIV, and b) a membrane domain that tethers the molecule to the membrane or facilitates the tethering of the molecule to the membrane. In some embodiments, the inhibitory moiety is selected from the group consisting of C34, HP32, SC35EK, sifuvirtide, T20, and T2634, or functional portions thereof. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, there is provided an engineered immune cell (e.g., a T cell) comprising a) an inhibitory moiety comprising the amino acid sequence of C34 (SEQ ID NO: 8) or a function portion thereof, and b) a membrane domain comprising a GPI attachment signal sequence (e.g., SEQ ID NO: 149), wherein the surface molecule does not comprise an intracellular signaling domain. In some embodiments, there is provided an engineered immune cell (e.g., a T cell) comprising a) an inhibitory moiety comprising the amino acid sequence of C34 (SEQ ID NO: 8) or a function portion thereof, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain.

In some embodiments, the V_{H} and the V_{L} described herein are fused via a linker. In some embodiments, the linker between the V_{H} and the V_{L} is a peptide linker. In some embodiments, the linker between the V_{H} and the V_{L} comprises the amino acid sequence of SEQ ID NO: 45. In some embodiments, the binding moiety is a sdAb, a scFv, a Fab′, a (Fab′)₂, an Fv, or a peptide ligand. In some embodiments, the binding moiety is a scFv.

In some embodiments, the binding moiety or the inhibitory moiety is fused to N-terminus of the membrane domain via a linker. In some embodiments, the linker between a) the binding moiety or inhibitory moiety and b) the membrane domain comprises a peptide linker. In some embodiments, the linker is selected from the group consisting of SEQ ID NOs: 45-52, 150 and 152.

In some embodiments, the engineered cell is a stem cell. In some embodiments, the stem cell is an embryonic stem cell (ESC). In some embodiments, the stem cell is hematopoietic stem cell (HSC). In some embodiments, the stem cell is a mesenchymal stem cell. In some embodiments, the stem cell is an induced pluripotent stem cell (iPSC).

In some embodiments, the engineered cell is an immune cell. In some embodiments, the immune cells is a T cells. In some embodiments, the immune cell is a B cell. In some embodiments, the immune cell is a Natural killer cell (NK cell).

In some embodiments, the engineered cell expresses a chimeric antigen receptor. In some embodiments, the chimeric antigen receptor comprises an anti-HIV antibody moiety (such as any of the anti-HIV antibody moieties described herein).

In some embodiments, the surface molecule comprises a second binding moiety that specifically binds to a second antigen. In some embodiments, the binding moiety and the second binding moiety are linked in tandem. In some embodiments, the surface molecule comprises a) an anti-CCR5 antibody moiety or anti-CD4 antibody moiety that specifically binds to CCR5 or CD4, and b) an anti-HIV antibody moiety that specifically binds to a HIV antigen or an inhibitory moiety that inhibits membrane fusion of HIV.

In some embodiments, the engineered cell comprises a second surface molecule, wherein the second surface molecule comprising a second binding moiety that specifically binds to a second antigen. In some embodiments, the engineered cell comprises a) an anti-CCR5 antibody moiety or anti-CD4 antibody moiety that specifically binds to CCR5 or CD4, and b) an anti-HIV surface molecule comprising an antibody moiety that specifically binds to an HIV antigen or an inhibitory moiety that inhibits membrane fusion of HIV.

The present application in another aspect provides an engineered cell expressing a surface molecule and exhibiting herd immunity against HIV. Herd immunity against HIV described herein refers to the phenomena that a cell expressing a certain surface molecule that prevents or inhibits the infection of HIV (such as any of the surface molecules described herein) is not only resistant to HIV infection itself, but also confers the anti-HIV immunity to another cell that is originally susceptible to HIV infection. Herd immunity against HIV can be tested by mixing a plurality of cells expressing the surface molecule with a plurality of cells not expressing the surface molecule and are susceptible to HIV infection at a certain ratio (such as 4:1, 3:1, 2:1, 1:1, 1:2, 1:3, 1:4), and then incubating the mixture of the cells with HIV. Herd immunity exists when the number of cells resistant to HIV infection after incubation is higher than (such as at least about 5%, 10%, 15%, or 20% higher than) the number of cells expressing the surface molecule. See e.g., Examples 2-5 for exemplary illustration of methods that can be used to test herd immunity effect. In some embodiments, the cell is a stem cell (such as a hematopoietic stem cell (HSC)). In some embodiments, the cell is an immune cell. In some embodiments, the cell is a T cell.

In some embodiments, there is provided a plurality of the engineered cell (such as a plurality of any of the engineered cell described herein), wherein upon a) mixture with a plurality of cells not expressing the surface molecule and are susceptible to HIV infection and b) contact of the cellular composition with an HIV, the percentage of cells not infected with the HIV is higher (such as at least about 10% higher) than the percentage of engineered cells. In some embodiments, the number of the plurality of cells not comprising a nucleic acid expressing the surface molecules between about 80% to about 120% (such as about 80%, 85%, 90%, 95%, 100%, 105%, 110%, 115% or 120%) of number of the plurality of engineered cells. In some embodiments, the plurality of cells not comprising a nucleic acid expressing the surface molecule express CCR5, CXCR4 and/or CD4 and are susceptible to HIV.

In some embodiments, there is provided a population of stem cells (e.g., hematopoietic stem cells) comprising a plurality of engineered stem cells (e.g., engineered hematopoietic stem cells) such as any of those described above, wherein purity of the engineered stem cell in the population of the stem cells is at least about 70%, 75%, 80%, 85%, 90%, 95%, 96%, 97%, 98%, or 99%. In some embodiments, the engineered stem cells express a surface molecule comprising an anti-CCR5 antibody moiety (e.g., an anti-CCR5 scFv). In some embodiments, the engineered stem cells express a surface molecule comprising an anti-CD4 antibody moiety (e.g., an anti-CD4 scFv). In some embodiments, the engineered stem cells express a surface molecule comprising an anti-HIV antibody moiety (e.g., an anti-HIV scFv, e.g., SEQ ID NO: 7, 61 or 62). In some embodiments, the engineered stem cells express a surface molecule comprising an inhibitory moiety (e.g., a C34 peptide, e.g., SEQ ID NO: 8).

### Surface Molecule

The present application also provides various surface molecules described herein. In some embodiments, the surface molecule comprises a) a binding moiety that prevents the binding of the HIV antigen to the engineered cell (such as any of the binding moieties described herein) and b) a membrane domain that tethers the molecule to the membrane or facilitates the tethering of the molecule to the membrane (such as any of the membrane domains described here, such as a GPI attachment signal sequence). In some embodiments, the surface molecule comprises a binding moiety that specifically binds to a T cell surface antigen (e.g., CCR5, CD4 or CXCR4) and prevents the binding of the T cell surface antigen to its cognitive ligand on HIV. In some embodiments, the surface molecule comprises a binding moiety that specifically binds a HIV antigen competitively with an anti-HIV antibody (such as 10-1074, 10E8, or PGT121), or binds to the same epitope as that of the anti-HIV antibody (such as 10-1074, 10E8, or PGT121) and prevents the binding of the HIV antigen to the engineered cell. In some embodiments, the surface molecule comprises an inhibitory moiety that inhibits membrane fusion of HIV and a membrane domain that tethers the molecule to the membrane or facilitates the tethering of the molecule to the membrane (such as a GPI attachment signal sequence). In some embodiments, the inhibitory moiety comprises the amino acid sequence of C34 (e.g., SEQ ID NO: 8) or a functional portion thereof.

In some embodiments, after the surface molecule is expressed by a cell, the cell confers herd immunity against HIV. In some embodiments, the cell expresses CCR5, CD4 or CXCR4. In some embodiments, the cell is a TZM-b1 cell.

In some embodiments, the binding moiety or the inhibitory moiety is fused to N-terminus of the membrane domain. In some embodiments, the binding moiety or the inhibitory moiety is fused to N-terminus of the membrane domain via a linker (such as any of the linkers described in the “linker” section). In some embodiments, the linker between a) the binding moiety or inhibitory moiety and b) the membrane domain comprises a peptide linker. In some embodiments, the linker is selected from the group consisting of SEQ ID NOs: 45-52, 150 and 152. In some embodiments, the binding moiety or the inhibitory moiety is fused to N-terminus of the membrane domain without a linker.

In some embodiments, the surface molecule does not comprise an intracellular signaling domain.

In some embodiments, the surface molecule further comprises a signal peptide at the N-terminus of the molecule that promotes the tethering of the surface molecule to the membrane. In some embodiments, the signal peptide is a CD8a signal peptide (e.g., SEQ ID NO: 148).

### Binding Moiety

The binding moieties described in this application can be either a) a binding moiety that specifically binds to a T cell surface antigen and prevents the binding of the T cell surface antigen to its cognitive ligand on HIV, or b) a binding moiety that specifically binds to a HIV antigen and prevents the binding of the HIV antigen to the engineered cell. In some embodiments, the binding moiety specifically binds to a T cell surface antigen, wherein the T cell surface antigen is selected from the group consisting of CCR5, CD4, and CXCR4. In some embodiments, the binding moiety that specifically binds to a HIV antigen competitively with 10-1074, 10E8, or PGT121, or binds to the same epitope as that of 10-1074, 10E8, or PGT121.

In some embodiments, the binding moiety is an antibody moiety that has a heavy chain variable region (V_{H}) and a light chain variable region (V_{L}), wherein the V_{H} comprises a HC-CDR1, a HC-CDR2, and a HC-CDR3, and the V_{L} comprises a LC-CDR1, a LC-CDR2, and a LC-CDR3. In some embodiments, the V_{H} and the V_{L} are fused via a linker (such as any of the linkers described in the “linker” section). In some embodiments, the linker between the V_{H} and the V_{L} is a peptide linker. In some embodiments, the linker between the V_{H} and the V_{L} comprises the amino acid sequence of SEQ ID NO: 45.

In some embodiments, the binding moiety is a sdAb, a scFv, a Fab′, a (Fab′)₂, an Fv, or a peptide ligand.

In some embodiments, the binding moiety is a scFv comprising a heavy chain variable region (V_{H}) and a light chain variable region (V_{L}). In some embodiments, the V_{H} is fused to the N-terminus of the V_{L}. In some embodiments, the V_{H} is fused to the C-terminus of the V_{L}. In some embodiments, the V_{H} and the V_{L} are fused via a linker (such as any of the linkers described in the “linker” section). In some embodiments, the linker between the V_{H} and the V_{L} is a peptide linker. In some embodiments, the linker between the V_{H} and the V_{L} comprises the amino acid sequence of SEQ ID NO: 45.

Exemplary binding moieties are described below.

### CCR5

In some embodiments, the T cell surface antigen is CCR5. In some embodiments, the binding moiety comprises an anti-CCR5 antibody moiety. In some embodiments, the binding moiety specifically binds to CCR5 competitively with C1-11, C1-12, C1-13, C1-14, C1-814, or C1-816, or specifically binds to the same epitope as that of C1-11, C1-12, C1-13, C1-14, C1-814, or C1-816.

In some embodiments, the binding moiety specifically binds to CCR5 competitively with C1-13, or specifically binds to the same epitope as that of C1-13. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14. In some embodiments, the binding moiety comprises the amino acid sequence of SEQ ID NO: 1, or a variant comprising an amino acid sequence having at least about 80% (such as at least about any one of 80%, 85%, 90%, 95%, 96%, 97%, 98%, or 99%) sequence identity.

In some embodiments, the binding moiety specifically binds to CCR5 competitively with C1-814, or specifically binds to the same epitope as that of C1-814. In some embodiments, the binding moiety specifically binds to CCR5 competitively with C1-816, or specifically binds to the same epitope as that of C1-816. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 15, the HC-CDR2 comprising the amino acid sequence of SEQ ID NO: 16, and the HC-CDR3 comprising the amino acid sequence of SEQ ID NO: 17, and the V_{L} comprises the LC-CDR1 comprising the amino acid sequence of SEQ ID NO: 18, the LC-CDR2 comprising the amino acid sequence of SEQ ID NO: 19, and the LC-CDR3 comprising the amino acid sequence of SEQ ID NO: 20. In some embodiments, the binding moiety comprises the amino acid sequence of SEQ ID NO: 2, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequence of SEQ ID NO: 2, or a variant comprising an amino acid sequence having at least about 80% (such as at least about any one of 80%, 85%, 90%, 95%, 96%, 97%, 98%, or 99%) sequence identity. In some embodiments, the binding moiety comprises the amino acid sequence of SEQ ID NO: 3, or a variant comprising an amino acid sequence having at least about 80% (such as at least about any one of 80%, 85%, 90%, 95%, 96%, 97%, 98%, or 99%) sequence identity.

### CD4

In some embodiments, the T cell surface antigen is CD4. In some embodiments, the binding moiety comprises an anti-CD4 antibody moiety. In some embodiments, the binding moiety specifically binds to CD4 competitively with C2-05, C2-11, or C2-13, or specifically binds to the same epitope as that of C2-05, C2-11, or C2-13.

In some embodiments, the binding moiety specifically binds to CD4 competitively with C2-05, or specifically binds to the same epitope as that of C2-05. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26. In some embodiments, the binding moiety comprises the amino acid sequence of SEQ ID NO: 4, or a variant comprising an amino acid sequence having at least about 80% (such as at least about any one of 80%, 85%, 90%, 95%, 96%, 97%, 98%, or 99%) sequence identity.

In some embodiments, the binding moiety specifically binds to CD4 competitively with C2-11, or specifically binds to the same epitope as that of C2-11. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 27, the HC-CDR2 comprising the amino acid sequence of SEQ ID NO: 28, and the HC-CDR3 comprising the amino acid sequence of SEQ ID NO: 29, and the V_{L} comprises the LC-CDR1 comprising the amino acid sequence of SEQ ID NO: 30, the LC-CDR2 comprising the amino acid sequence of SEQ ID NO: 31, and the LC-CDR3 comprising the amino acid sequence of SEQ ID NO: 32. In some embodiments, the binding moiety comprises the amino acid sequence of SEQ ID NO: 5, or a variant comprising an amino acid sequence having at least about 80% (such as at least about any one of 80%, 85%, 90%, 95%, 96%, 97%, 98%, or 99%) sequence identity.

In some embodiments, the binding moiety specifically binds to CD4 competitively with C2-13, or specifically binds to the same epitope as that of C2-13. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38. In some embodiments, the binding moiety comprises the amino acid sequence of SEQ ID NO: 6, or a variant comprising an amino acid sequence having at least about 80% (such as at least about any one of 80%, 85%, 90%, 95%, 96%, 97%, 98%, or 99%) sequence identity.

### Anti-HIV Binding Moiety

In some embodiments, the binding moiety specifically binds to a HIV antigen (e.g., an anti-HIV antibody). See e.g., Kwong et al., Immunity. 2018 May 15; 48(5):855-871; Sok et al., Nat Immunol. 2018 November; 19(11):1179-1188; West Jr. et al., Cell. 2014 Feb. 13; 156(4): 633-648 for exemplary anti-HIV antibodies.

In some embodiments, the binding moiety specifically binds to a CD4 targeting site on HIV. In some embodiments, the binding moiety specifically binds to HIV competitively with B12, IOMA, NIH45, NIH46, 12A12, VRC-PG04, VRC-CH31, VRC13.01, VRC16.01, 8ANC131, B2530, CH103, N6, N49-P7, VRC01, CH235.12, NC-Cowl, IOMA, CH235, 3BNC117, or HJ16, or specifically binds to the same epitope as that of B12, IOMA, NIH45, NIH46, 12A12, VRC-PG04, VRC-CH31, VRC13.01, VRC16.01, 8ANC131, B2530, CH103, N6, N49-P7, VRC01, CH235.12, NC-Cowl, IOMA, CH235, 3BNC117, or HJ16. In some embodiments, the binding moiety comprises the HIV binding moiety of B12, IOMA, NIH45, NIH46, 12A12, VRC-PG04, VRC-CH31, VRC13.01, VRC16.01, 8ANC131, B2530, CH103, N6, N49-P7, VRC01, CH235.12, NC-Cowl, IOMA, CH235, 3BNC117, or HJ16, or a functional variant thereof.

In some embodiments, the binding moiety specifically binds to glycan V3 (such as Asn332 glycan-V3) on HIV. In some embodiments, the binding moiety specifically binds to HIV competitively with PGT121, BG18, DH270.6, PDGM12, PGDM21, PCDN-33A, DH270.1, VRC41.01, BF520.1, VRC29.03, PGT128, PGT135, or 2G12, or specifically binds to the same epitope as that of PGT121, BG18, DH270.6, PDGM12, PGDM21, PCDN-33A, DH270.1, VRC41.01, BF520.1, VRC29.03, PGT128, PGT135, or 2G12. In some embodiments, the binding moiety comprises the HIV binding moiety of PGT121, BG18, DH270.6, PDGM12, PGDM21, PCDN-33A, DH270.1, VRC41.01, BF520.1, VRC29.03, PGT128, PGT135, or 2G12, or a functional variant thereof.

In some embodiments, the binding moiety specifically binds to glycan V1/V2 (such as Asn160 glycan-V1/V2) on HIV. In some embodiments, the binding moiety specifically binds to HIV competitively with PGDM1400, VRC26.25, CH01, CH02, CH03, CH-04, PCT64-24E, VRC38.01, PG9, PG16, PGT141, PGT142, PGT143, PGT144, or PGT145, or specifically binds to the same epitope as that of PGDM1400, VRC26.25, CH01, CH02, CH03, CH-04, PCT64-24E, VRC38.01, PG9, PG16, PGT141, PGT142, PGT143, PGT144, or PGT145. In some embodiments, the binding moiety comprises the HIV binding moiety of PGDM1400, VRC26.25, CH01, CH02, CH03, CH-04, PCT64-24E, VRC38.01, PG9, PG16, PGT141, PGT142, PGT143, PGT144, or PGT145, or a functional variant thereof.

In some embodiments, the binding moiety specifically binds to interface or fusion peptide on HIV. In some embodiments, the binding moiety specifically binds to HIV competitively with PGT151, VRC34.01, 35O22, or ACS202, or specifically binds to the same epitope as that of PGT151, VRC34.01, 35O22, or ACS202. In some embodiments, the binding moiety comprises the HIV binding moiety of PGT151, VRC34.01, 35O22, or ACS202, or a functional variant thereof.

In some embodiments, the binding moiety specifically binds to silent face on HIV. In some embodiments, the binding moiety specifically binds to HIV competitively with VRC-PG05, or specifically binds to the same epitope as that of VRC-PG05. In some embodiments, the binding moiety comprises the HIV binding moiety of VRC-PG05, or a functional variant thereof.

In some embodiments, the binding moiety specifically binds to the membrane-proximal external region (MPER) on HIV. In some embodiments, the binding moiety specifically binds to HIV competitively with 4E10, 2F5, Z13, DF511, Z13e1, DH511.11P, or M66.6, or specifically binds to the same epitope as that of 4E10, 2F5, Z13, DF511, Z13e1, DH511.11P, or M66.6. In some embodiments, the binding moiety comprises the HIV binding moiety of 4E10, 2F5, Z13, DF511, Z13e1, DH511.11P, or M66.6, or a functional variant thereof.

In some embodiments, the binding moiety specifically binds to HIV competitively with 8ANC195, 3BC176, or X5, or specifically binds to the same epitope as that of 8ANC195, 3BC176, or X5. In some embodiments, the binding moiety comprises the HIV binding moiety of 8ANC195, 3BC176, or X5, or a functional variant thereof.

In some embodiments, the binding moiety specifically binds to HIV competitively with 10-1074, 10E8, or PGT121 specifically binds to the same epitope as that of 10-1074, 10E8, or PGT121.

In some embodiments, the binding moiety specifically binds to HIV competitively with 10-1074, or specifically binds to the same epitope as that of 10-1074. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 39, the HC-CDR2 comprising the amino acid sequence of SEQ ID NO: 40, and the HC-CDR3 comprising the amino acid sequence of SEQ ID NO: 41, and the V_{L} comprises the LC-CDR1 comprising the amino acid sequence of SEQ ID NO: 42, the LC-CDR2 comprising the amino acid sequence of SEQ ID NO: 43, and the LC-CDR3 comprising the amino acid sequence of SEQ ID NO: 44. In some embodiments, the binding moiety comprises the amino acid sequences of SEQ ID NO: 7, or a variant comprising an amino acid sequence having at least about 80% (such as at least about any one of 80%, 85%, 90%, 95%, 96%, 97%, 98%, or 99%) sequence identity.

In some embodiments, the binding moiety that specifically binds to HIV competitively with 10E8, or specifically binds to the same epitope as that of 10E8. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 63, the HC-CDR2 comprising the amino acid sequence of SEQ ID NO: 64, and the HC-CDR3 comprising the amino acid sequence of SEQ ID NO: 65, and the V_{L} comprises the LC-CDR1 comprising the amino acid sequence of SEQ ID NO: 66, the LC-CDR2 comprising the amino acid sequence of SEQ ID NO: 67, and the LC-CDR3 comprising the amino acid sequence of SEQ ID NO: 68. In some embodiments, the binding moiety comprises the amino acid sequences of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, the binding moiety that specifically binds to HIV competitively with PGT121, or specifically binds to the same epitope as that of PGT121. In some embodiments, the binding moiety comprises an antibody moiety comprising a heavy chain variable region (V_{H}) and a light chain variable region (V_{L}), wherein the V_{H} comprises the HC-CDR1 comprising the amino acid sequence of SEQ ID NO: 69, the HC-CDR2 comprising the amino acid sequence of SEQ ID NO: 70, and the HC-CDR3 comprising the amino acid sequence of SEQ ID NO: 71, and the V_{L} comprises the LC-CDR1 comprising the amino acid sequence of SEQ ID NO: 72, the LC-CDR2 comprising the amino acid sequence of SEQ ID NO: 73, and the LC-CDR3 comprising the amino acid sequence of SEQ ID NO: 74. In some embodiments, the binding moiety comprises the amino acid sequences of SEQ ID NO: 62, or a variant comprising an amino acid sequence having at least about 80% (such as at least about any one of 80%, 85%, 90%, 95%, 96%, 97%, 98%, or 99%) sequence identity.

Inhibitory Moiety that Inhibits Membrane Fusion of HIV

Inhibitory moieties described herein inhibit membrane fusion of HIV. In some embodiments, the inhibitory moiety targets gp41. Exemplary inhibitory moieties include C34, HP32, SC35EK, sifuvirtide, T20, T2634 or functional portions thereof. See e.g., Woodham et al. AIDS Patient Care STDS. 2016 Jul. 1; 30(7): 291-306.

In some embodiments, the inhibitory moiety comprises the amino acid sequence of C34 or a functional portion thereof. In some embodiments, the inhibitory moiety comprises the amino acid sequence of SEQ ID NO: 8.

### Membrane Domain

Membrane domains described in this application include any molecule that is capable of a) tethering the binding moiety or inhibitory moiety described herein to the membrane of a cell or b) facilitating the tethering of the binding moiety or inhibitory moiety to the membrane.

In some embodiments, the membrane domain comprises a GPI attachment signal sequence (e.g., SEQ ID NO: 149). In some embodiments, the GPI attachment signal sequence comprises the amino acid sequence of SEQ ID NO: 149.

In some embodiments, the membrane domain is derived from or is a transmembrane domain from CD4, CD8, CD28, 4-1BB, or PD-1.

In some embodiments, the membrane domain is not fused with an intracellular signaling domain.

### Multispecific Surface Molecule

The present application also provide multispecific surface molecules described herein. In some embodiments, the surface molecule described herein comprises two or more moieties (e.g., two or more binding moieties as described herein, two or more inhibitory moieties described herein, or one or more binding moieties and one or more inhibitory moieties).

In some embodiments, the surface molecule comprises a first binding moiety that specifically binds to a first antigen, and a second binding moiety that specifically binds to a second antigen, wherein both first antigen and second antigen are T cell surface antigens, wherein both binding moieties prevents the binding of the T cell surface antigen to its cognitive ligand on HIV, and wherein both the first binding moiety and the second binding moiety are tethered to cell membrane via a membrane domain. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, the surface molecule comprises a first binding moiety that specifically binds to CCR5, and a second binding moiety that specifically binds to CD4, wherein both the first binding moiety and the second binding moiety are tethered to cell membrane via a membrane domain. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain. In some embodiments, the first binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the first binding moiety comprises the amino acid sequence of any one of SEQ ID NOs: 1-3 and 133-135, or a variant comprising an amino acid sequence having at least about 80% (such as at least about any one of 80%, 85%, 90%, 95%, 96%, 97%, 98%, or 99%) sequence identity. In some embodiments, the second binding moiety specifically binds to CD4 competitively with C2-05, C2-11, or C2-13, or specifically binds to the same epitope as that of C2-05, C2-11, or C2-13. In some embodiments, the second binding moiety comprises the amino acid sequences of SEQ ID NO: 4, or a variant comprising an amino acid sequence having at least about 80% (such as at least about any one of 80%, 85%, 90%, 95%, 96%, 97%, 98%, or 99%) sequence identity.

In some embodiments, the surface molecule comprises a first binding moiety that specifically binds to CCR5, and a second binding moiety that specifically binds to CD4, wherein the surface molecule further comprise a membrane domain comprising a GPI attachment signal sequence (e.g., SEQ ID NO: 149). In some embodiments, the first binding moiety comprises the amino acid sequence of SEQ ID NO: 1, or a variant comprising an amino acid sequence having at least about 80% (such as at least about any one of 80%, 85%, 90%, 95%, 96%, 97%, 98%, or 99%) sequence identity. In some embodiments, the first binding moiety comprises the amino acid sequence of SEQ ID NO: 6, or a variant comprising an amino acid sequence having at least about 80% (such as at least about any one of 80%, 85%, 90%, 95%, 96%, 97%, 98%, or 99%) sequence identity.

In some embodiments, the surface molecule comprises a first binding moiety that specifically binds to CCR5, and a second binding moiety that specifically binds to CXCR4, wherein the surface molecule further comprises a membrane domain that tethers the molecule to the membrane or facilitates the tethering of the molecule to the membrane. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain. In some embodiments, the first binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the first binding moiety comprises the amino acid sequence of any one of SEQ ID NOs: 1-3 and 133-135 (e.g., SEQ ID NO: 1), or a variant comprising an amino acid sequence having at least about 80% (such as at least about any one of 80%, 85%, 90%, 95%, 96%, 97%, 98%, or 99%) sequence identity.

In some embodiments, the surface molecule comprises a first binding moiety that specifically binds to CD4, and a second binding moiety that specifically binds to CXCR4, wherein the surface molecule further comprises a membrane domain that tethers the molecule to the membrane or facilitates the tethering of the molecule to the membrane. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain. In some embodiments, the first binding moiety specifically binds to CD4 competitively with C2-05, C2-11, or C2-13, or specifically binds to the same epitope as that of C2-05, C2-11, or C2-13. In some embodiments, the first binding moiety comprises the amino acid sequences of any of SEQ ID NOs: 4-6 (e.g., SEQ ID NO: 6), or a variant comprising an amino acid sequence having at least about 80% (such as at least about any one of 80%, 85%, 90%, 95%, 96%, 97%, 98%, or 99%) sequence identity.

In some embodiments, the surface molecule comprises a first binding moiety that specifically binds to a T cell surface antigen (e.g., CCR5, CD4, CXCR4) and prevents the binding of the T cell surface antigen to its cognitive ligand on HIV, and a second binding moiety that specifically binds to a HIV antigen and prevents the binding of the HIV antigen to the engineered cell, and wherein the surface molecule further comprises a membrane domain that tethers the molecule to the membrane or facilitates the tethering of the molecule to the membrane. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, the surface molecule comprises a first binding moiety that specifically binds to CCR5, and a second binding moiety that specifically binds to a HIV antigen and prevents the binding of the HIV antigen to the engineered cell, and wherein the surface molecule further comprises a membrane domain that tethers the molecule to the membrane or facilitates the tethering of the molecule to the membrane. In some embodiments, the second binding moiety specifically binds to a HIV antigen competitively with 10-1074, 10E8, or PGT121, or binds to the same epitope as that of 10-1074, 10E8, or PGT121. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain. In some embodiments, the first binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the first binding moiety comprises the amino acid sequence of any one of SEQ ID NOs: 1-3 and 133-135 (e.g., SEQ ID NO: 1), or a variant comprising an amino acid sequence having at least about 80% (such as at least about any one of 80%, 85%, 90%, 95%, 96%, 97%, 98%, or 99%) sequence identity.

In some embodiments, the surface molecule comprises a first binding moiety that specifically binds to CD4, and a second binding moiety that specifically binds to a HIV antigen and prevents the binding of the HIV antigen to the engineered cell, and wherein the surface molecule further comprises a membrane domain that tethers the molecule to the membrane or facilitates the tethering of the molecule to the membrane. In some embodiments, the second binding moiety specifically binds to a HIV antigen competitively with 10-1074, 10E8, or PGT121, or binds to the same epitope as that of 10-1074, 10E8, or PGT121. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain. In some embodiments, the first binding moiety specifically binds to CD4 competitively with C2-05, C2-11, or C2-13, or specifically binds to the same epitope as that of C2-05, C2-11, or C2-13. In some embodiments, the first binding moiety comprises the amino acid sequences of any of SEQ ID NOs: 4-6 (e.g., SEQ ID NO: 6), or a variant comprising an amino acid sequence having at least about 80% (such as at least about any one of 80%, 85%, 90%, 95%, 96%, 97%, 98%, or 99%) sequence identity.

In some embodiments, the surface molecule comprises a binding moiety that specifically binds to a T cell surface antigen (e.g., CCR5, CD4, CXCR4) and prevents the binding of the T cell surface antigen to its cognitive ligand on HIV, and an inhibitory moiety that inhibits membrane fusion of HIV, wherein the surface molecule further comprises a membrane domain that tethers the molecule to the membrane or facilitates the tethering of the molecule to the membrane. In some embodiments, the inhibitory moiety comprises the amino acid sequence of HP32, SC35EK, sifuvirtide, T20, or T2634, or a functional portion thereof. In some embodiments, the inhibitory moiety comprises the amino acid sequence of C34 or a functional portion thereof. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, the surface molecule comprises a binding moiety that specifically binds to CCR5, and an inhibitory moiety that inhibits membrane fusion of HIV comprising the amino acid sequence of C34 or a functional portion thereof (e.g., SEQ ID NO: 8), wherein the surface molecule further comprises a membrane domain that tethers the molecule to the membrane or facilitates the tethering of the molecule to the membrane. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain. In some embodiments, the binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the binding moiety comprises the amino acid sequence of any one of SEQ ID NOs: 1-3 and 133-135 (e.g., SEQ ID NO: 1), or a variant comprising an amino acid sequence having at least about (such as at least about any one of 80%, 85%, 90%, 95%, 96%, 97%, 98%, or 99%) 80% sequence identity.

In some embodiments, the surface molecule comprises a binding moiety that specifically binds to CCR5 comprising the amino acid sequence of SEQ ID NO: 1, and an inhibitory moiety that inhibits membrane fusion of HIV comprising the amino acid sequence of SEQ ID NO: 8, wherein the surface molecule further comprises a membrane domain that tethers the molecule to the membrane or facilitates the tethering of the molecule to the membrane (e.g., a GPI attachment signal sequence), wherein the surface molecule does not have an intracellular domain.

In some embodiments, the surface molecule comprises a binding moiety that specifically binds to a HIV antigen and prevents the binding of the HIV antigen to the engineered cell, and an inhibitory moiety that inhibits membrane fusion of HIV, wherein the surface molecule further comprises a membrane domain that tethers the molecule to the membrane or facilitates the tethering of the molecule to the membrane. In some embodiments, the binding moiety binds to a HIV antigen competitively with 10-1074, 10E8, or PGT121, or binds to the same epitope as that of 10-1074, 10E8, or PGT121 and prevents the binding of the HIV antigen to the engineered cell. In some embodiments, the surface molecule comprises a binding moiety that specifically binds to HIV competitively with 10-1074, or specifically binds to the same epitope as that of 10-1074. In some embodiments, the binding moiety comprises the amino acid sequences of SEQ ID NO: 7, or a variant comprising an amino acid sequence having at least about 80% (such as at least about any one of 80%, 85%, 90%, 95%, 96%, 97%, 98%, or 99%) sequence identity. In some embodiments, the inhibitory moiety comprises the amino acid sequence of HP32, SC35EK, sifuvirtide, T20, or T2634, or a functional portion thereof. In some embodiments, the inhibitory moiety comprises the amino acid sequence of C34 or a functional portion thereof (e.g., SEQ ID NO: 8). In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, the surface molecule comprises a binding moiety that specifically binds to a HIV antigen comprising the amino acid sequences of SEQ ID NO: 7 and an inhibitory moiety comprising the amino acid sequence of SEQ ID NO: 8, wherein the surface molecule further comprises a membrane domain that tethers the molecule to the membrane or facilitates the tethering of the molecule to the membrane (e.g., a GPI attachment signal sequence). In some embodiments, the surface molecule does not comprise an intracellular signaling domain.

In some embodiments, the surface molecule comprises a first binding moiety and a second binding moiety, wherein the first binding moiety and the second binding moiety bind to two distinct epitopes of a same antigen, where the antigen is a) a T cell surface antigen selected from the group consisting of CCR5, CD4, and CXCR4 or b) a HIV antigen, wherein the surface molecule further comprises a membrane domain that tethers the molecule to the membrane or facilitates the tethering of the molecule to the membrane (e.g., a GPI signal attachment sequence).

Engineered Cells Comprising One or More Nucleic Acids that Encode Two or More Distinct Surface Molecules

The present application also provides engineered cells comprising one or more nucleic acids that encode two or more distinct surface molecules. Various embodiments discussed in the “multispecific surface molecule” section are also conceived in the context of engineered cells comprising one or more nucleic acids that encode two or more distinct surface molecules for the same desired effects.

In some embodiments, the engineered cell comprises one or more nucleic acid encoding a) a first surface molecule comprising a first binding moiety that specifically binds to a first antigen fused to a first membrane domain, and b) a second surface molecule comprising a second binding moiety that specifically binds to a second antigen fused to a second membrane domain, wherein the first and the second membrane domain tether the first binding moiety and the second binding moiety to membrane or facilitate the tethering of the two moieties to the membrane, respectively, wherein both first antigen and second antigen are T cell surface antigens, wherein both binding moieties prevents the binding of the T cell surface antigen to its cognitive ligand on HIV, In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain.

In some embodiments, the engineered cell comprises one or more nucleic acid encoding a) a first surface molecule comprising a first binding moiety that specifically binds to CCR5 fused to a first membrane domain, and b) a second surface molecule comprising a second binding moiety that specifically binds to CD4 fused to a second membrane domain, wherein the first and the second membrane domain tether the first binding moiety and the second binding moiety to membrane or facilitate the tethering of the two moieties to the membrane, respectively. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain. In some embodiments, the first binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the first binding moiety comprises the amino acid sequence of any one of SEQ ID NOs: 1-3 and 133-135, or a variant comprising an amino acid sequence having at least about 80% (such as at least about any one of 80%, 85%, 90%, 95%, 96%, 97%, 98%, or 99%) sequence identity. In some embodiments, the second binding moiety specifically binds to CD4 competitively with C2-05, C2-11, or C2-13, or specifically binds to the same epitope as that of C2-05, C2-11, or C2-13. In some embodiments, the second binding moiety comprises the amino acid sequences of any of SEQ ID NOs: 4-6, or a variant comprising an amino acid sequence having at least about 80% (such as at least about any one of 80%, 85%, 90%, 95%, 96%, 97%, 98%, or 99%) sequence identity.

In some embodiments, the engineered cell comprises one or more nucleic acid encoding a) a first surface molecule comprising a first binding moiety that specifically binds to CCR5 fused to a first membrane domain, and a second surface molecule comprising a second binding moiety that specifically binds to CD4 fused to a second membrane domain, wherein the first and the second membrane domain tether the first binding moiety and the second binding moiety to membrane or facilitate the tethering of the two moieties to the membrane, respectively. In some embodiments, the first and/or the second membrane domain comprising a GPI attachment signal sequence (e.g., SEQ ID NO: 149). In some embodiments, the first binding moiety comprises the amino acid sequence of SEQ ID NO: 1 or 6, or a variant comprising an amino acid sequence having at least about 80% (such as at least about any one of 80%, 85%, 90%, 95%, 96%, 97%, 98%, or 99%) sequence identity.

In some embodiments, the engineered cell comprises one or more nucleic acid encoding a) a first surface molecule comprising a first binding moiety that specifically binds to CCR5 fused to a first membrane domain, and b) a second surface molecule comprising a second binding moiety that specifically binds to CXCR4 fused to a second membrane domain, wherein the first and the second membrane domain tether the first binding moiety and the second binding moiety to membrane or facilitate the tethering of the two moieties to the membrane, respectively. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, first and/or the second the surface molecule does not comprise an intracellular signaling domain. In some embodiments, the first binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the first binding moiety comprises the amino acid sequence of any one of SEQ ID NOs: 1-3 and 133-135 (e.g., SEQ ID NO: 1), or a variant comprising an amino acid sequence having at least about 80% (such as at least about any one of 80%, 85%, 90%, 95%, 96%, 97%, 98%, or 99%) sequence identity.

In some embodiments, the engineered cell comprises one or more nucleic acid encoding a) a first surface molecule comprising a first binding moiety that specifically binds to CD4 fused to a first membrane domain, and b) a second surface molecule comprising a second binding moiety that specifically binds to CXCR4 fused to a second membrane domain, wherein the first and the second membrane domain tether the first binding moiety and the second binding moiety to membrane or facilitate the tethering of the two moieties to the membrane, respectively. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain. In some embodiments, the first binding moiety specifically binds to CD4 competitively with C2-05, C2-11, or C2-13, or specifically binds to the same epitope as that of C2-05, C2-11, or C2-13. In some embodiments, the first binding moiety comprises the amino acid sequences of any of SEQ ID NOs: 4-6 (e.g., SEQ ID NO: 6), or a variant comprising an amino acid sequence having at least about 80% (such as at least about any one of 80%, 85%, 90%, 95%, 96%, 97%, 98%, or 99%) sequence identity.

In some embodiments, the engineered cell comprises one or more nucleic acid encoding a) a first surface molecule comprising a first binding moiety that specifically binds to a T cell surface antigen (e.g., CCR5, CD4, CXCR4) and prevents the binding of the T cell surface antigen to its cognitive ligand on HIV, wherein the first binding moiety is fused to a first membrane domain, and b) a second surface molecule comprising a second binding moiety that specifically binds to a HIV antigen and prevents the binding of the HIV antigen to the engineered cell, wherein the second binding domain is fused to a second membrane domain, and wherein the first and the second membrane domain tether the first binding moiety and the second binding moiety to membrane or facilitate the tethering of the two moieties to the membrane, respectively. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain.

In some embodiments, the engineered cell comprises one or more nucleic acid encoding a) a first surface molecule comprising a first binding moiety that specifically binds to CCR5 fused to a first membrane domain, and b) a second surface molecule comprising a second binding moiety that specifically binds to a HIV antigen and prevents the binding of the HIV antigen to the engineered cell, wherein the second binding moiety is fused to a second membrane domain, and wherein the first and the second membrane domain tether the first binding moiety and the second binding moiety to membrane or facilitate the tethering of the two moieties to the membrane, respectively. In some embodiments, the second binding moiety specifically binds to a HIV antigen competitively with 10-1074, 10E8, or PGT121, or binds to the same epitope as that of 10-1074, 10E8, or PGT121. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain. In some embodiments, the first binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the first binding moiety comprises the amino acid sequence of any one of SEQ ID NOs: 1-3 and 133-135 (e.g., SEQ ID NO: 1), or a variant comprising an amino acid sequence having at least about 80% (such as at least about any one of 80%, 85%, 90%, 95%, 96%, 97%, 98%, or 99%) sequence identity.

In some embodiments, the engineered cell comprises one or more nucleic acid encoding a) a first surface molecule comprising a first binding moiety that specifically binds to CD4 fused to a first membrane domain, and b) a second surface molecule comprising a second binding moiety that specifically binds to a HIV antigen and prevents the binding of the HIV antigen to the engineered cell, wherein the second binding moiety is fused to a second membrane domain, and wherein the first and the second membrane domain tether the first binding moiety and the second binding moiety to membrane or facilitate the tethering of the two moieties to the membrane, respectively. In some embodiments, the second binding moiety specifically binds to a HIV antigen competitively with 10-1074, 10E8, or PGT121, or binds to the same epitope as that of 10-1074, 10E8, or PGT121. In some embodiments, the first and/or second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain. In some embodiments, the first binding moiety specifically binds to CD4 competitively with C2-05, C2-11, or C2-13, or specifically binds to the same epitope as that of C2-05, C2-11, or C2-13. In some embodiments, the first binding moiety comprises the amino acid sequences of any of SEQ ID NOs: 4-6 (e.g., SEQ ID NO: 6), or a variant comprising an amino acid sequence having at least about 80% (such as at least about any one of 80%, 85%, 90%, 95%, 96%, 97%, 98%, or 99%) sequence identity.

In some embodiments, the engineered cell comprises one or more nucleic acid encoding a) a first surface molecule comprising a binding moiety that specifically binds to a T cell surface antigen (e.g., CCR5, CD4, CXCR4) and prevents the binding of the T cell surface antigen to its cognitive ligand on HIV, wherein the binding moiety is fused to a first membrane domain, and b) a second surface molecule comprising an inhibitory moiety that inhibits membrane fusion of HIV, wherein the inhibitory moiety is fused to a second membrane domain, wherein the first and the second membrane domain tether the first binding moiety and the second binding moiety to membrane or facilitate the tethering of the two moieties to the membrane, respectively. In some embodiments, the inhibitory moiety comprises the amino acid sequence of HP32, SC35EK, sifuvirtide, T20, or T2634, or a functional portion thereof. In some embodiments, the inhibitory moiety comprises the amino acid sequence of C34 or a functional portion thereof. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the surface molecule does not comprise an intracellular signaling domain.

In some embodiments, the engineered cell comprises one or more nucleic acid encoding a) a first surface molecule comprising a binding moiety that specifically binds to CCR5 fused to a first membrane domain, and b) a second surface molecule comprising an inhibitory moiety that inhibits membrane fusion of HIV comprising the amino acid sequence of C34 or a functional portion thereof (e.g., SEQ ID NO: 8) fused to a second membrane domain, wherein the first and the second membrane domain tether the first binding moiety and the second binding moiety to membrane or facilitate the tethering of the two moieties to the membrane, respectively. In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain. In some embodiments, the binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816. In some embodiments, the binding moiety comprises the amino acid sequence of any one of SEQ ID NOs: 1-3 and 133-135 (e.g., SEQ ID NO: 1), or a variant comprising an amino acid sequence having at least about (such as at least about any one of 80%, 85%, 90%, 95%, 96%, 97%, 98%, or 99%) 80% sequence identity.

In some embodiments, the engineered cell comprises one or more nucleic acid encoding a) a first surface molecule comprising a binding moiety that specifically binds to CCR5 comprising the amino acid sequence of SEQ ID NO: 1 fused to a first membrane domain, and b) a second surface molecule comprising an inhibitory moiety that inhibits membrane fusion of HIV comprising the amino acid sequence of SEQ ID NO: 8 fused to a second membrane domain, wherein the first and the second membrane domain tether the first binding moiety and the second binding moiety to membrane or facilitate the tethering of the two moieties to the membrane, respectively. In some embodiments, the first and/or the second membrane domain is a GPI attachment signal sequence (e.g., SEQ ID NO: 149). In some embodiments, wherein the first and/or the second surface molecule does not have an intracellular domain.

In some embodiments, the engineered cell comprises one or more nucleic acid encoding a) a first surface molecule comprising a binding moiety that specifically binds to a HIV antigen and prevents the binding of the HIV antigen to the engineered cell, wherein the binding moiety is fused to a first membrane domain, and b) a second surface molecule comprising an inhibitory moiety that inhibits membrane fusion of HIV fused to a second membrane domain, wherein the first and the second membrane domain tether the first binding moiety and the second binding moiety to membrane or facilitate the tethering of the two moieties to the membrane, respectively. In some embodiments, the binding moiety binds to a HIV antigen competitively with 10-1074, 10E8, or PGT121, or binds to the same epitope as that of 10-1074, 10E8, or PGT121 and prevents the binding of the HIV antigen to the engineered cell. In some embodiments, the first surface molecule comprises a binding moiety that specifically binds to HIV competitively with 10-1074, or specifically binds to the same epitope as that of 10-1074. In some embodiments, the binding moiety comprises the amino acid sequences of SEQ ID NO: 7, or a variant comprising an amino acid sequence having at least about 80% (such as at least about any one of 80%, 85%, 90%, 95%, 96%, 97%, 98%, or 99%) sequence identity. In some embodiments, the inhibitory moiety comprises the amino acid sequence of HP32, SC35EK, sifuvirtide, T20, or T2634, or a functional portion thereof. In some embodiments, the inhibitory moiety comprises the amino acid sequence of C34 or a functional portion thereof (e.g., SEQ ID NO: 8). In some embodiments, the first and/or the second membrane domain comprises a GPI attachment signal sequence (e.g., SEQ ID NO: 149). In some embodiments, the first and/or the second membrane domain is derived from or is a transmembrane domain from CD4, CD8, CD28, 4-1BB, or PD-1. In some embodiments, the first and/or the second surface molecule does not comprise an intracellular signaling domain.

In some embodiments, the engineered cell comprises one or more nucleic acid encoding a) a first surface molecule comprising a binding moiety that specifically binds to a HIV antigen comprising the amino acid sequences of SEQ ID NO: 7 fused to a first membrane domain, and a second surface molecule comprising an inhibitory moiety comprising the amino acid sequence of SEQ ID NO: 8 fused to a second membrane domain, wherein both the binding moiety and the inhibitory moiety are tethered to cell membrane via the first and the second membrane domains (e.g., a GPI anchor). In some embodiments, the first and/or the second surface molecule does not comprise an intracellular signaling domain.

In some embodiments, the engineered cell comprises one or more nucleic acid encoding a) a first surface molecule comprising a first binding moiety fused to a first membrane domain, and b) a second surface molecule comprising a second binding moiety fused to a second membrane domain, and wherein the first binding moiety and the second binding moiety bind to two distinct epitopes of a same antigen, where the antigen is a) a T cell surface antigen selected from the group consisting of CCR5, CD4, and CXCR4 or b) a HIV antigen, wherein the first and the second membrane domain tether the first binding moiety and the second binding moiety to membrane or facilitate the tethering of the two moieties to the membrane. In some embodiments, the first and/or the second membrane domain comprises a GPI attachment signal sequence (e.g., SEQ ID NO: 149).

### Linker

The length, the degree of flexibility and/or other properties of the linker(s) used in the surface molecules (e.g., the linker between the V_{H} and the V_{L} of the binding moieties, e.g., the linker between the binding/inhibitory moiety and the membrane domain, e.g., the linker that links two moieties (e.g., two binding moieties) in bispecific surface molecules) may have some influence on properties, including but not limited to the affinity, specificity or avidity for one or more particular antigens or epitopes. For example, longer linkers may be selected to ensure that two adjacent domains do not sterically interfere with one another. In some embodiments, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

In some embodiments, the linker is a peptide linker as described below. In some embodiments, the peptide linker has a length of about one to about fifty, about two to about fourth, about three to about thirty, or about four to about twenty amino acids.

In some embodiments, the linker is a GS linker.

In some embodiments, the linker comprises an amino acid sequence of any one of SEQ ID NOs: 45-60. In some embodiments, the linker comprises an amino acid sequence of any one of SEQ ID NOs: 45-52 and 59-60. In some embodiments, the linker comprises an amino acid sequence of any one of SEQ ID NOs: 53-58.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 49), glycine-serine polymers (including, for example, (GS)ₙ (SEQ ID NO: 50), (GSGGS)ₙ (SEQ ID NO: 51), (GGGGS)ₙ (SEQ ID NO: 46), and (GGGS)ₙ (SEQ ID NO: 52, where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure. In some embodiments, the linker is a GS linker. In some embodiments, the linker has an amino acid sequence selected from the group consisting of SEQ ID NOs: 45-52 and 59-60. In some embodiments, the peptide linker comprises the hinge region of an IgG, such as the hinge region of human IgG1. In some embodiments, the linker has a sequence of an amino acid sequence selected from the group consisting of SEQ ID NOs: 53-58.

### Non-Peptide Linkers

Coupling of two moieties may be accomplished by any chemical reaction that will bind the two molecules so long as both components retain their respective activities. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers the can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus may lead to bispecific antibodies with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### Methods of Preparation

Also provided are compositions and methods for preparing the engineered cells described herein.

### Antibody Moieties

In some embodiments, the binding moieties described herein comprise antibody moieties (for example anti-CCR5 antibody moiety, anti-CD4 antibody moiety, anti-CXCR4 antibody moiety, anti-HIV antibody moiety). In some embodiments, the antibody moiety comprises V_{H} and V_{L} domains, or variants thereof, from the monoclonal antibody. In some embodiments, the antibody moiety further comprises CH1 and CL domains, or variants thereof, from the monoclonal antibody. Monoclonal antibodies can be prepared, e.g., using hybridoma methods, such as those described by Kohler and Milstein, Nature, 256:495 (1975) and Sergeeva et al., Blood, 117(16):4262-4272.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest, or a complex comprising at least two molecules, such as a complex comprising a peptide and an MHC protein. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies. Kozbor, J. Immunol., 133:3001 (1984); Brodeur et al. Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (MA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107: 220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, the antibody moiety comprises sequences from a clone selected from an antibody moiety library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibody moiety can be prepared using phage display to screen libraries for antibodies specific to the target antigen (such as a CCR5, CXCR4, and CD4 polypeptides). The library can be a human scFv phage display library having a diversity of at least one×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully synthetic phage display library. In some embodiments, the library is a non-human phage display library.

Phage clones that bind to the target antigen with high affinity can be selected by iterative binding of phage to the target antigen, which is bound to a solid support (such as, for example, beads for solution panning or mammalian cells for cell panning), followed by removal of non-bound phage and by elution of specifically bound phage. In an example of solution panning, the target antigen can be biotinylated for immobilization to a solid support. The biotinylated target antigen is mixed with the phage library and a solid support, such as streptavidin-conjugated Dynabeads M-280, and then target antigen-phage-bead complexes are isolated. The bound phage clones are then eluted and used to infect an appropriate host cell, such as E. coli XL1-Blue, for expression and purification. In an example of cell panning, cells expressing the target antigen (e.g., CD4, CCR5) are mixed with the phage library, after which the cells are collected and the bound clones are eluted and used to infect an appropriate host cell for expression and purification. The panning can be performed for multiple (such as about any of 2, 3, 4, 5, 6 or more) rounds with either solution panning, cell panning, or a combination of both, to enrich for phage clones binding specifically to the target antigen. Enriched phage clones can be tested for specific binding to the target antigen by any methods known in the art, including for example ELISA and FACS.

In some embodiments, the binding moieties bind to the same epitope as a reference antibody. In some embodiments, the binding moieties compete for binding with a reference antibody. Competition assays can be used to determine whether two antibodies moieties bind the same epitope (or compete with each other) by recognizing identical or sterically overlapping epitopes or one antibody competitively inhibits binding of another antibody to the antigen. Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to the same epitope if each blocks binding of the other by 50% or more.

### Human and Humanized Antibody Moieties

The antibody moieties described herein can be human or humanized. Humanized forms of non-human (e.g., murine) antibody moieties are chimeric immunoglobulins, immunoglobulin chains, or fragments thereof (such as Fv, Fab, Fab′, F(ab′)2, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody moiety nor in the imported CDR or framework sequences. In general, the humanized antibody moiety can comprise substantially all of at least one, and typically two, variable domains, in which all or substantially all of the CDR regions correspond to those of a non-human immunoglobulin, and all or substantially all of the FR regions are those of a human immunoglobulin consensus sequence. See, e.g., Jones et al., Nature, 321: 522-525 (1986); Riechmann et al., Nature, 332: 323-329 (1988); Presta, Curr. Op. Struct. Biol., 2:593-596 (1992).

Generally, a humanized antibody moiety has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody moiety. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Antibody Variants

In some embodiments, amino acid sequence variants of the antigen-binding domains (e.g., anti-CCR5 antibody moiety, anti-CD4 antibody moiety, and anti-HIV antibody moieties) provided herein are contemplated. For example, it may be desirable to improve the binding affinity and/or other biological properties of the antigen-binding domain. Amino acid sequence variants of an antigen-binding domain may be prepared by introducing appropriate modifications into the nucleotide sequence encoding the antigen-binding domain, or by peptide synthesis. Such modifications include, for example, deletions from, and/or insertions into and/or substitutions of residues within the amino acid sequences of the antigen-binding domain. Any combination of deletion, insertion, and substitution can be made to arrive at the final construct, provided that the final construct possesses the desired characteristics, e.g., antigen-binding.

In some embodiments, antigen-binding domain variants having one or more amino acid substitutions are provided. Sites of interest for substitutional mutagenesis include the HVRs and FRs of antibody moieties. Amino acid substitutions may be introduced into an antigen-binding domain of interest and the products screened for a desired activity, e.g., retained/improved antigen binding or decreased immunogenicity.

Conservative substitutions are shown in Table 2 below. Variant CORS discussed herein can also contain such conservative substitutions.

TABLE 2
CONSERVATIVE SUBSTITITIONS

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f aromatic: Trp, Tyr, Phe.

Non-conservative substitutions will entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g., binding affinity). Alterations (e.g., substitutions) may be made in HVRs, e.g., to improve antibody moiety affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HC-CDR3 and LC-CDR3 in particular are often targeted.

In some embodiments, substitutions, insertions, or deletions may occur within one or more HVRs so long as such alterations do not substantially reduce the ability of the antibody moiety to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antigen-binding domain that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antigen-binding domain with antigen is affected. Further substitutions may be introduced at the amino acid locations demonstrating functional sensitivity to the initial substitutions. Alternatively, or additionally, a crystal structure of an antigen-antigen-binding domain complex can be determined to identify contact points between the antigen-binding domain and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding domain with an N-terminal methionyl residue. Other insertional variants of the antigen-binding domain include the fusion to the N- or C-terminus of the antigen-binding domain to an enzyme (e.g., for ADEPT) or a polypeptide which increases the serum half-life of the antigen-binding domain.

### Nucleic Acids

Also provided herein are nucleic acids (or a set of nucleic acids) encoding the surface molecules, binding moieties, inhibitory moieties, membrane domains described herein, as well as vectors comprising the nucleic acid(s).

The expression of the surface molecules, binding moieties, inhibitory moieties, membrane domains can be achieved by inserting the nucleic acid(s) into an appropriate expression vector, such that the nucleic acid(s) is operably linked to 5′ and/or 3′ regulatory elements, including for example a promoter (e.g., a lymphocyte-specific promoter) and a 3′ untranslated region (UTR). The vectors can be suitable for replication and integration in host cells. Typical cloning and expression vectors contain transcription and translation terminators, initiation sequences, and promoters useful for regulation of the expression of the desired nucleic acid sequence.

The nucleic acid(s) can be cloned into a number of types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art. Viruses that are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatinine kinase promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, (3-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Exemplary methods to confirm the presence of the nucleic acid(s) in the mammalian cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

For example, in some embodiments, the nucleic acid comprises a first nucleic acid sequence encoding a first surface molecule comprising a binding moiety that specifically binds to a T cell surface antigen (e.g., CCR5, CD4, CXCR4), optionally a second nucleic acid encoding a second surface molecule comprising an inhibitory domain that inhibits membrane fusion of HIV (such as C34), optionally a third nucleic acid encoding a third surface molecule comprising a binding moiety that specifically binds to a HIV antigen. In some embodiments, the first nucleic acid sequence is contained in a first vector, the optional second nucleic acid sequence is contained in a second vector, and the optional third nucleic acid sequence is contained in a third vector. In some embodiments, the first and second nucleic acid sequences are contained in a first vector, and the third nucleic acid sequence is contained in a second vector. In some embodiments, the first and third nucleic acid sequences are contained in a first vector, and the second nucleic acid sequence is contained in a second vector. In some embodiments, the second and third nucleic acid sequences are contained in a first vector, and the first nucleic acid sequence is contained in a second vector. In some embodiments, the first, second, and third nucleic acid sequences are contained in the same vector. In some embodiments, the first, second, and third nucleic acids can be connected to each other via a linker selected from the group consisting of an internal ribosomal entry site (IRES) and a nucleic acid encoding a self-cleaving 2A peptide (such as P2A, T2A, E2A, or F2A).

In some embodiments, the first nucleic acid sequence is under the control of a first promoter, the optional second nucleic acid sequence is under the control of a second promoter, and the optional third nucleic acid sequence is under the control of a third promoter. In some embodiments, some or all of the first, second, and third promoters have the same sequence. In some embodiments, some or all of the first, second, and third promoters have different sequences. In some embodiments, some or all of the first, second, and third, nucleic acid sequences are expressed as a single transcript under the control of a single promoter in a multicistronic vector. In some embodiments, one or more of the promoters are inducible.

In some embodiments, some or all of the first, second, and third nucleic acid sequences have similar (such as substantially or about the same) expression levels in a cell (such as a stem cell, such as an immune cell, such as a T cell). In some embodiments, some of the first, second, and third nucleic acid sequences have expression levels in a cell (such as a stem cell, such as an immune cell, such as a T cell) that differ by no more than 2 times. Expression can be determined at the mRNA or protein level. The level of mRNA expression can be determined by measuring the amount of mRNA transcribed from the nucleic acid using various well-known methods, including Northern blotting, quantitative RT-PCR, microarray analysis and the like. The level of protein expression can be measured by known methods including immunocytochemical staining, enzyme-linked immunosorbent assay (ELISA), western blot analysis, luminescent assays, mass spectrometry, high performance liquid chromatography, high-pressure liquid chromatography-tandem mass spectrometry, and the like.

Methods of introducing and expressing genes into in a cell (such as a stem cell, such as an immune cell, such as a T cell) are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human, cells. Other viral vectors can be derived from lentivirus, poxviruses, herpes simplex virus 1, adenoviruses and adeno-associated viruses, and the like.

Chemical means for introducing a polynucleotide into a host cell (such as immune cell) include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances that may be naturally occurring or synthetic lipids. For example, lipids include the fatty droplets that naturally occur in the cytoplasm as well as the class of compounds that contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The nucleic acids described herein may be transiently or stably incorporated in a cell (such as a stem cell, such as an immune cell, such as a T cell). In some embodiments, the nucleic acid is transiently expressed in the engineered cell. For example, the nucleic acid may be present in the nucleus of the engineered cell in an extrachromosomal array comprising the heterologous gene expression cassette. Nucleic acids may be introduced into the engineered mammalian using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the nucleic acid is a DNA. In some embodiments, the nucleic acid is a RNA. In some embodiments, the nucleic acid is linear. In some embodiments, the nucleic acid is circular.

In some embodiments, the nucleic acid(s) is present in the genome of the engineered cell. For example, the nucleic acid(s) may be integrated into the genome of the cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN′), CRISPR/Cas9, and zinc-finger nucleases. In some embodiments, the nucleic acid(s) is integrated in a specifically designed locus of the genome of the engineered cell. In some embodiments, the nucleic acid(s) is integrated in an integration hotspot of the genome of the engineered cell. In some embodiments, the nucleic acid(s) is integrated in a random locus of the genome of the engineered cell. In the cases that multiple copies of the nucleic acids are present in a single engineered cell, the nucleic acid(s) may be integrated in a plurality of loci of the genome of the engineered cell.

The nucleic acid(s) encoding the surface molecule(s) can be operably linked to a promoter. In some embodiments, the promoter is an endogenous promoter. For example, the nucleic acid(s) encoding the surface molecule(s) may be knocked-in to the genome of the engineered cell downstream of an endogenous promoter using any methods known in the art, such as CRISPR/Cas9 method. In some embodiments, the endogenous promoter is a promoter for an abundant protein, such as beta-actin, CMV, or EF1α. In some embodiments, the endogenous promoter is an inducible promoter, for example, inducible by an endogenous activation signal of the engineered cell. In some embodiments, wherein the engineered cell is a T cell, the promoter is a T cell activation-dependent promoter (such as an IL-2 promoter, an NFAT promoter, or an NFκB promoter).

In some embodiments, the promotor is an autologous promoter.

In some embodiments, the promoter is a heterologous promoter.

In some embodiments, the nucleic acid(s) encoding the surface molecule(s) is operably linked to a constitutive promoter. In some embodiments, the nucleic acid(s) encoding the surface molecule is operably linked to an inducible promoter. In some embodiments, a first promoter (e.g., an inducible promoter) is operably linked to a nucleic acid encoding a first surface molecule comprising an anti-HIV antibody moiety or inhibitory moiety that inhibits membrane fusion of HIV, and a second consecutive promoter (e.g., a consecutive promoter) is operably linked to a nucleic acid encoding a second surface molecule comprising an antibody moiety that specifically binds to a T cell surface antigen (e.g., CD4, CCR5, CXCR4), or vice versa.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken (3-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the promoter in the nucleic acid is a hEF1α promoter.

The inducible promoter can be induced by one or more conditions, such as a physical condition, microenvironment of the engineered cell, or the physiological state of the engineered cell, an inducer (i.e., an inducing agent), or a combination thereof. In some embodiments, the inducing condition does not induce the expression of endogenous genes in the engineered cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most mature system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a HIV antigen. In some embodiments, the polypeptide is expressed by the engineered cell. In some embodiments, the polypeptide is encoded by a nucleic acid in the nucleic acid. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly put into two categories based on their regulations of (1) DNA binding or (2) recruitment of a transcriptional activation domain to a DNA bound protein. For instance, synthetic mammalian blue light controlled transcription system based on melanopsin, which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG (SEQ ID NO: 165) have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015. Any of the immediate early gene promoters or any promoter comprising a serum response element or SEQ ID NO: 65 may be useful as a radiation inducible promoter to drive the expression of the therapeutic protein of the present invention.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, it was shown that HIF-2α restricts HIV transcription via direct binding to the viral promoter. Hypoxia reduced tumor necrosis factor or histone deacetylase inhibitor, Romidepsin, mediated reactivation of HIV and inhibiting HIF signaling-pathways reversed this phenotype. (See Zhuang et al., Commun Biol 3, 376 (2020)).

In some embodiments, the promoter is inducible by the physiological state, such as an endogenous activation signal, of the engineered cell. In some embodiments, wherein the engineered cell is a stem cell (e.g., hematopoietic stem cell), the promoter is a stem cell (e.g., hematopoietic stem cell) activation-dependent promoter. In some embodiments, wherein the engineered cell is a T cell, the promoter is a T cell activation-dependent promoter, which is inducible by the endogenous activation signal of the engineered T cell. In some embodiments, the engineered T cell is activated by an inducer, such as PMA, ionomycin, or phytohaemagglutinin. In some embodiments, the engineered T cell is activated by recognition of HIV antigen via an endogenous T cell receptor, or an engineered receptor (such as recombinant TCR, or CAR). In some embodiments, the engineered T cell is activated by blockade of an immune checkpoint, such as by an immunomodulator expressed by the engineered T cell or by a second engineered cell. In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

Without being bound by any theory or hypothesis, IL-2 expression initiated by the gene transcription from IL-2 promoter is a major activity of T cell activation. Un-specific stimulation of human T cells by Phorbol 12-myristate 13-acetate (PMA), or ionomycin, or phytohaemagglutinin results in IL-2 secretion from stimulated T cells. IL-2 promoter was explored for activation-induced transgene expression in genetically engineered T-cells (Virology Journal 3:97 (2006)). We found that IL-2 promoter is efficient to initiate reporter gene expression in the presence of PMA/PHA-P activation in human T cell lines. T cell receptor stimulation initiates a cascade of intracellular reactions causing an increasing of cytosolic calcium concentrations and resulting in nuclear translation of both NFAT and NFκB. Members of Nuclear Factor of Activated T cells (NFAT) are Ca′ dependent transcription factors mediating immune response in T lymphocytes. NFAT have been shown to be crucial for inducible interleukine-2 (IL-2) expression in activated T cells (Mol Cell Biol. 15(11):6299-310 (1995); Nature Reviews Immunology 5:472-484 (2005)). We found that NFAT promoter is efficient to initiate reporter gene expression in the presence of PMA/PHA-P activation in human T cell lines. Other pathways including nuclear factor kappa B (NFκB) can also be employed to control transgene expression via T cell activation.

### Preparation of Engineered Cells

In some embodiments, the engineered cells are stem cells. In some embodiments, the stem cells are hematopoietic stem cells (e.g., CD34+ and/or CD33− cells). The stem cells may be derived from placental cells, embryonic stem cells, induced pluripotent stem cells, or hematopoietic stem cells. The hematopoietic stem cells can be obtained from bone marrow cells or peripheral blood mononuclear cells (PBMCs).

In some embodiments, the engineered cells are immune cells.

The engineered immune cells may be obtained from peripheral blood, cord blood, bone marrow, tumor infiltrating lymphocytes, lymph node tissue, or thymus tissue. The host cells may include placental cells, embryonic stem cells, induced pluripotent stem cells, or hematopoietic stem cells. The cells may be obtained from humans, monkeys, chimpanzees, dogs, cats, mice, rats, and transgenic species thereof. The cells may be obtained from established cell lines.

In some embodiments, the engineered immune cell is derived from a stem cell. In some embodiments, the stem cell is an embryonic stem cell (ESC). In some embodiments, the stem cell is hematopoietic stem cell (HSC). In some embodiments, the stem cell is a mesenchymal stem cell. In some embodiments, the stem cell is an induced pluripotent stem cell (iPSC).

The engineered cells expressing the surface molecule can be generated by introducing one or more nucleic acids (including for example a lentiviral vector) encoding the surface molecule into the cell. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The nucleic acid can be inserted into a vector and packaged in retroviral particles using techniques known in the art. The recombinant virus can then be isolated and delivered to the engineered cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the nucleic acid sequence(s) encoding the surface molecule can be packaged with protocols known in the art. The resulting lentiviral vectors can be used to transduce a mammalian cell (such as primary human T cells) using methods known in the art.

In some embodiments, the transduced or transfected mammalian cell is propagated ex vivo after introduction of the nucleic acid. In some embodiments, the transduced or transfected mammalian cell is cultured to propagate for at least about any of 1 day, 2 days, 3 days, 4 days, 5 days, 6 days, 7 days, 10 days, 12 days, or 14 days. In some embodiments, the transduced or transfected mammalian cell is cultured for no more than about any of 1 day, 2 days, 3 days, 4 days, 5 days, 6 days, 7 days, 10 days, 12 days, or 14 days. In some embodiments, the transduced or transfected mammalian cell is further evaluated or screened to select the engineered cell.

The introduction of the one or more nucleic acids into the cell (e.g., a stem cell or an immune cell) can be accomplished using techniques known in the art. In some embodiments, the engineered cells (such as engineered stem cell or T cells) are able to self-renew, expand and/or differentiate in vivo, resulting in long-term persistence that can lead to sustained control of a disease.

In some embodiments, prior to genetic modification and/or expanding of the cells, a source of the cells is obtained from a subject. The cells (e.g., stem cells or immune cells) can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of cell lines available in the art may be used. In some embodiments of the present invention, cells can be obtained from a unit of blood or bone marrow collected from a subject using any number of techniques known to the skilled artisan, such as FICOLL™ separation. In some embodiments, cells from the circulating blood or bone marrow of an individual are obtained by apheresis.

Stem cells can be obtained from an adult or neonatal umbilical cord whole leukocyte sample or whole bone marrow cell sample. The hematopoietic stem cells can be derived from a source selected from the group consisting of bone marrow, peripheral blood, and neonatal umbilical cord blood. In some embodiments, the stem cells are human hematopoietic stem cells selected based upon one or more of the following markers: CD34+, CD59+, CD90/Thy1+, CD38low/−, c-Kit−/low, and Lin−. In some embodiments, the stem cells are mouse hematopoietic stem cells based upon one or more of the following markers: CD34low/−, SCA-1+, CD90/Thy1+/low, CD38+, c-Kit+, and Lin−.

Methods of culturing, enriching and expanding stem cells are well appreciated in the field, for example, as described in Bernstein et al., Stem Cell Res Ther. 2012; 3(3): 17, Kôhler et al., Stem Cells, 17: 19-24, and WO2003062369A2, which are incorporated herein by their entireties.

Immune cells (e.g., T cells) can be derived from apheresis products. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, immune cells (such as T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. Longer incubation times may be used to isolate T cells in any situation where there are few T cells as compared to other cell types. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells, which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments, the immune cells (such as T cells) described herein are expanded by contacting with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

Whether prior to or after genetic modification of the cells (e.g., stem cells, e.g., immune cells) to express the surface molecule(s), the cells can be activated and expanded.

### Genetic Modifications

In some embodiments, the engineered cell is modified to block or decrease the expression of CCR5. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In some embodiments, engineered cells with reduced expression of CCR5 are generated using the CRISPR/Cas system. For a review of the CRISPR/Cas system of gene editing, see for example Jian W & Marraffini L A, Annu. Rev. Microbiol. 69, 2015; Hsu P D et al., Cell, 157(6):1262-1278, 2014; and O'Connell M R et al., Nature 516: 263-266, 2014. In some embodiments, Engineered T cells with reduced expression of one or both of the endogenous TCR chains of the T cell are generated, for example using TALEN-based genome editing. In some embodiments, the engineered cells, in particular allogeneic immune cells obtained from donors can be modified to inactivate components of TCR involved in MHC recognition. In some embodiments, the modified immune cells do not cause graft versus host disease.

In some embodiments, the CCR5 gene (or TCR gene) is inactivated using CRISPR/Cas9 gene editing. CRISPR/Cas9 involves two main features: a short guide RNA (gRNA) and a CRISPR-associated endonuclease or Cas protein. The Cas protein is able to bind to the gRNA, which contains an engineered spacer that allows for directed targeting to, and subsequent knockout of, a gene of interest. Once targeted, the Cas protein cleaves the DNA target sequence, resulting in the knockout of the gene.

In some embodiments, the CCR5 gene (or TCR gene) is inactivated using transcription activator-like effector nuclease (TALEN®)-based genome editing. TALEN®-based genome editing involves the use of restriction enzymes that can be engineered for targeting to particular regions of DNA. A transcription activator-like effector (TALE) DNA-binding domain is fused to a DNA cleavage domain. The TALE is responsible for targeting the nuclease to the sequence of interest, and the cleavage domain (nuclease) is responsible for cleaving the DNA, resulting in the removal of that segment of DNA and subsequent knockout of the gene.

In some embodiments, the CCR5 gene (or TCR gene) is inactivated using zinc finger nuclease (ZFN) genome editing methods. Zinc finger nucleases are artificial restriction enzymes that are comprised of a zinc finger DNA-binding domain and a DNA-cleavage domain. ZFN DNA-binding domains can be engineered for targeting to particular regions of DNA. The DNA-cleavage domain is responsible for cleaving the DNA sequence of interest, resulting in the removal of that segment of DNA and subsequent knockout of the gene.

In some embodiments, the expression of the CCR5 gene is reduced by using RNA interference (RNAi) such as small interference RNA (siRNA), microRNA, and short hairpin RNA (shRNA). siRNA molecules are 20-25 nucleotide long oligonucleotide duplexes that are complementary to messenger RNA (mRNA) transcripts from genes of interest. siRNAs target these mRNAs for destruction. Through targeting, siRNAs prevent mRNA transcripts from being translated, thereby preventing the protein from being produced by the cell.

In some embodiments, the expression of the CCR5 gene (or TCR gene) is reduced by using anti-sense oligonucleotides. Antisense oligonucleotides targeting mRNA are generally known in the art and used routinely for downregulating gene expressions. See Watts, J. and Corey, D (2012) J. Pathol. 226(2):365-379.)

### Enrichment of the Engineered Cells

In some embodiments, there is provided a method of enriching a heterogeneous cell population for engineered cells expressing a surface molecule according to any of the engineered cells described herein.

In some embodiments, there is provided a method of enriching a population for engineered cells comprising a nucleic acid encoding a surface molecule that comprises a) a binding moiety (such as any of the binding moieties described herein) or a inhibitory moiety (such as any of the inhibitory moieties described herein) and b) a membrane domain, wherein the method comprises enrichment based upon the binding moiety or the inhibitory moiety. In some embodiments, the binding moiety specifically binds to CCR5, CD4 or CXCR4. In some embodiments the binding moiety specifically binds to a HIV antigen. In some embodiments, the inhibitory moiety comprises a C34 peptide. In some embodiments, the method comprises incubating an agent that specifically binds to the binding moiety or inhibitory moiety with the population of the engineered cells. In some embodiments, the method comprises incubating an agent that specifically binds to the membrane domain. In some embodiments, the agent is an antibody. In some embodiments, at least about 90%, 95%, 96%, 97%, 98%, or 99% purity of cells is achieved via enrichment method described herein.

A population of engineered cells produced according to the methods described herein can be enriched for by positive selection techniques based upon, e.g., the expression of the surface molecule(s). For example, in some embodiments, engineered cells (such as engineered stem cells or T cells) are enriched for by incubation with target antigen-conjugated beads and/or target ligand-conjugated beads for a time period sufficient for positive selection of the desired engineered cells, wherein the beads target the binding domain or inhibitory domain on the surface molecule. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of engineered cells present at low levels in the heterogeneous cell population, use of longer incubation times, such as 24 hours, can increase cell yield. Longer incubation times may be used to isolate engineered cells in any situation where there are few engineered cells as compared to other cell types. The skilled artisan would recognize that multiple rounds of selection can also be used in the context of this invention.

For isolation of a desired population of engineered cells by positive selection, the concentration of cells and the beads can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of engineered cells that may weakly express the surface molecule(s).

In some embodiments, enrichment results in minimal or substantially no exhaustion of the engineered cells (e.g., immune cells). For example, in some embodiments, enrichment results in fewer than about 50% (such as fewer than about any of 45, 40, 35, 30, 25, 20, 15, 10, or 5%) of the engineered cells becoming exhausted. Immune cell exhaustion can be determined by any means known in the art, including any means described herein.

In some embodiments, enrichment results in minimal or substantially no differentiation of the engineered cells (e.g., stem cells, e.g., immune cells). For example, in some embodiments, enrichment results in fewer than about 50% (such as fewer than about any of 45, 40, 35, 30, 25, 20, 15, 10, or 5%) of the engineered cells becoming differentiated. Cell (e.g., stem cells, e.g., immune cells) differentiation can be determined by any methods known in the art, including any methods described herein.

In some embodiments, enrichment results in minimal or substantially no internalization of surface molecule(s) on the engineered cells. For example, in some embodiments, enrichment results in less than about 50% (such as less than about any of 45, 40, 35, 30, 25, 20, 15, 10, or 5%) of the surface molecule(s) on the engineered cells becoming internalized. Internalization of the surface molecule(s) on engineered cells can be determined by any methods known in the art, including any methods described herein.

In some embodiments, enrichment results in increased proliferation of the engineered cells. For example, in some embodiments, enrichment results in an increase of at least about 10% (such as at least about any of 20, 30, 40, 50, 60, 70, 80, 90, 100, 200, 300, 400, 500, 1000% or more) in the number of engineered cells following enrichment.

Thus, in some embodiments, there is provided a method of enriching a heterogeneous cell population for engineered cells expressing a surface molecule comprising: a) contacting the heterogeneous cell population with a molecule comprising a target molecule (such as CCR5, CD4, an HIV antigen) or one or more epitopes contained therein to form complexes comprising the engineered cell bound to the molecule comprising the engineered cell bound to the molecule; and b) separating the complexes from the heterogeneous cell population, thereby generating a cell population enriched for the engineered cells. In some embodiments, the molecule is immobilized, individually, to a solid support. In some embodiments, the solid support is particulate (such as beads). In some embodiments, the solid support is a surface (such as the bottom of a well). In some embodiments, the molecule is labelled, individually, with a tag. In some embodiments, the tag is a fluorescent molecule, an affinity tag, or a magnetic tag. In some embodiments, the method further comprises eluting the engineered cells from the first and/or second molecules and recovering the eluate.

In some embodiments, the engineered immune cells are enriched for CD4+ and/or CD8+ cells, for example through the use of negative enrichment, whereby cell mixtures are purified using two-step purification methods involving both physical (column) and magnetic (MACS magnetic beads) purification steps (Gunzer, M. et al. (2001) J. Immunol. Methods 258(1-2):55-63). In other embodiments, populations of cells can be enriched for CD4+ and/or CD8+ cells through the use of T cell enrichment columns specifically designed for the enrichment of CD4+ or CD8+ cells. In yet other embodiments, cell populations can be enriched for CD4+ cells through the use of commercially available kits. In some embodiments, the commercially available kit is the EASYSEP™ Human CD4+ T Cell Enrichment Kit (Stemcell Technologies). In other embodiments, the commercially available kit is the MAGNISORT™ Mouse CD4+ T cell Enrichment Kit (Thermo Fisher Scientific).

### Pharmaceutical Compositions

Also provided herein are engineered cell compositions (such as pharmaceutical compositions, also referred to herein as formulations) comprising an engineered cell (such as a stem cell, such as a T cell) described herein.

In some embodiments, there is provided an engineered cell composition comprising a homogeneous cell population of engineered cells (such as a stem cell, such as a T cell) of the same cell type, comprising one or more nucleic acids encoding same surface molecule(s), and optionally expressing the same surface molecule(s). In some embodiments, the engineered cell is a stem cell (e.g., hematopoietic stem cell). In some embodiments, the engineered cell is a T cell. In some embodiments, the engineered cell is selected from the group consisting of a cytotoxic T cell, a helper T cell, a natural killer T cell, and a γδT cell. In some embodiments, the engineered cell composition is a pharmaceutical composition.

In some embodiments, there is provided an engineered cell composition comprising a heterogeneous cell population comprising a plurality of engineered cell populations comprising engineered cells of different cell types, which comprise different nucleic acids encoding different surface molecules, and optionally expressing different surface molecules.

In some embodiments, the pharmaceutical composition is suitable for administration to an individual, such as a human individual. In some embodiments, the pharmaceutical composition is suitable for injection. In some embodiments, the pharmaceutical composition is suitable for infusion. In some embodiments, the pharmaceutical composition is substantially free of cell culture medium. In some embodiments, the pharmaceutical composition is substantially free of endotoxins or allergenic proteins. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is free of mycoplasma, microbial agents, and/or communicable disease agents.

The pharmaceutical composition of the present applicant may comprise any number of the engineered cells. In some embodiments, the pharmaceutical composition comprises a single copy of the engineered cell. In some embodiments, the pharmaceutical composition comprises at least about any of 1, 10, 100, 1000, 10⁴, 10⁵, 10⁶, 10⁷, 10⁸ or more copies of the engineered cells. In some embodiments, the pharmaceutical composition comprises a single type of engineered cell. In some embodiments, the pharmaceutical composition comprises at least two types of engineered cells, wherein the different types of engineered cells differ by their cell sources, cell types, expressed therapeutic proteins (e.g., surface molecule(s)), and/or promoters, etc.

At various points during preparation of a composition, it can be necessary or beneficial to cryopreserve a cell. The terms “frozen/freezing” and “cryopreserved/cryopreserving” can be used interchangeably. Freezing includes freeze-drying.

In some embodiments, cells can be harvested from a culture medium, and washed and concentrated into a carrier in a therapeutically effective amount. Exemplary carriers include saline, buffered saline, physiological saline, water, Hanks' solution, Ringer's solution, Nonnosol-R (Abbott Labs), Plasma-Lyte A(R) (Baxter Laboratories, Inc., Morton Grove, IL), glycerol, ethanol, and combinations thereof.

In some embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients, which can range in function from a bulking agent to an additive, which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells, including any values and ranges in between these values.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the nucleic acids encoding one or more surface molecules described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water. The amounts of such additives and aqueous vehicles to be added can be suitably selected according to the form of use of the nucleic acid composition.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

### Excipient

The pharmaceutical compositions of the present application are useful for therapeutic purposes. Thus, different from other compositions comprising engineered cells, such as production cells that express the surface molecule, the pharmaceutical compositions of the present application comprises a pharmaceutically acceptable excipient suitable for administration to an individual.

Suitable pharmaceutically acceptable excipient may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, the pharmaceutically acceptable excipient comprises autologous serum. In some embodiments, the pharmaceutically acceptable excipient comprises human serum. In some embodiments, the pharmaceutically acceptable excipient is non-toxic, biocompatible, non-immunogenic, biodegradable, and can avoid recognition by the host's defense mechanism. The excipient may also contain adjuvants such as preserving stabilizing, wetting, emulsifying agents and the like. In some embodiments, the pharmaceutically acceptable excipient enhances the stability of the engineered cell or the antibody or other therapeutic proteins secreted thereof. In some embodiments, the pharmaceutically acceptable excipient reduces aggregation of the antibody or other therapeutic proteins secreted by the engineered cell. The final form may be sterile and may also be able to pass readily through an injection device such as a hollow needle. The proper viscosity may be achieved and maintained by the proper choice of excipients.

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

In some embodiments, the pharmaceutical composition is suitable for administration to a human. In some embodiments, the pharmaceutical composition is suitable for administration to a human by parenteral administration. Formulations suitable for parenteral administration include aqueous and non-aqueous, isotonic sterile injection solutions, which can contain antioxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a condition requiring only the addition of the sterile liquid excipient methods of treatment, methods of administration, and dosage regimens described herein (i.e., water) for injection, immediately prior to use. In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition is formulated for intravenous administration. In some embodiments, the pharmaceutical composition is formulated for subcutaneous administration. In some embodiments, the pharmaceutical composition is formulated for local administration to a tumor site. In some embodiments, the pharmaceutical composition is formulated for intratumoral injection.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the engineered mammalian immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the engineered cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the engineered cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### Methods of Treating Diseases Using Engineered Cells

The present application further provides methods of administering the engineered cells to treat an infectious disease, for example HIV. The present application thus in some embodiments provides a method for treating an infectious disease in an individual comprising administering to the individual an effective amount of a composition (such as a pharmaceutical composition) comprising engineered cells according to any one of the embodiments described herein. In some embodiments, the viral infection is caused by a virus selected, for example, Human T cell leukemia virus (HTLV) and HIV (Human immunodeficiency virus).

In some embodiments, methods of treating HIV are provided, which comprise administering any of the engineered cells described herein. There are two subtypes of HIV: HIV-1 and HIV-2. HIV-1 is the cause of the global pandemic and is a virus with both high virulence and high infectivity. HIV-2, however, is prevalent only in West Africa and is neither as virulent nor as infectious as HIV-1. The differences in virulence and infectivity between HIV-1 and HIV-2 infections may be rooted in the stronger immune response mounted against viral proteins in HIV-2 infections leading to more efficient control in affected individuals (Leligdowicz, A. et al. (2007) J. Clin. Invest. 117(10):3067-3074). This may also be a controlling reason for the global spread of HIV-1 and the limited geographic prevalence of HIV-2.

Although HIV-2 infections are better controlled than HIV-1 infections, HIV-2-affected individuals still benefit from treatment. In some embodiments, the engineered cells are used for treating HIV-1 infections. In other embodiments, the engineered cells are used for treating HIV-2 infections. In some embodiments, the engineered cells are used for treating HIV-1 and HIV-2 infections.

In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to a T cell surface antigen and prevents the binding of the T cell surface antigen to its cognitive ligand on HIV, wherein the T cell surface antigen is selected from the group consisting of CCR5, CD4, and CXCR4, and b) a membrane domain that tethers the molecule to the membrane or facilitates the tethering of the molecule to the membrane. In some embodiments, the surface molecule does not comprise an intracellular signaling domain.

In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to CCR5, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to CCR5, and b) a membrane domain comprising a GPI attachment signal sequence (e.g., SEQ ID NO: 149), wherein the surface molecule does not comprise an intracellular signaling domain. In some embodiments, the binding moiety is a scFv comprising the amino acid sequence of SEQ ID NO: 1, 2, or 3, or a variant comprising at least about 80% sequence identity.

In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to CD4, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to CD4, and b) a membrane domain comprising a GPI attachment signal sequence (e.g., SEQ ID NO: 149), wherein the surface molecule does not comprise an intracellular signaling domain. In some embodiments, the binding moiety is a scFv comprising the amino acid sequence of SEQ ID NO: 4, 5, or 6, or a variant comprising at least about 80% sequence identity.

In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to CXCR4, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to CXCR4, and b) a membrane domain comprising a GPI attachment signal sequence (e.g., SEQ ID NO: 149), wherein the surface molecule does not comprise an intracellular signaling domain.

In some embodiments, there is provided a method of treating an individual infected with HIV, comprising administering to the individual an effective amount of engineered stem cells (e.g., hematopoietic stem cells) comprising a) a binding moiety, wherein the binding moiety specifically binds to a HIV antigen and prevents the binding of the HIV antigen to the engineered cell, and b) a membrane domain that tethers the molecule to the membrane or facilitates the tethering of the molecule to the membrane. In some embodiments, the surface molecule does not comprise an intracellular signaling domain. In some embodiments, the binding moiety competitively with 10-1074, 10E8, or PGT121, or binds to the same epitope as that of 10-1074, 10E8, or PGT121. In some embodiments, the binding moiety is a scFv comprising the amino acid sequence of SEQ ID NO: 7, 61, or 62, or a variant comprising at least about 80% sequence identity.

In some embodiments, there is provided a method of treating an individual infected with HIV, comprising administering to the individual an effective amount of engineered stem cells (e.g., hematopoietic stem cells) comprising a) an inhibitory moiety that inhibits membrane fusion of HIV, and b) a membrane domain that tethers the molecule to the membrane or facilitates the tethering of the molecule to the membrane. In some embodiments, the inhibitory moiety is selected from the group consisting of C34, HP32, SC35EK, sifuvirtide, T20, and T2634, or functional portions thereof. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, there is provided a method of treating an individual infected with HIV, comprising administering to the individual an effective amount of engineered stem cells (e.g., hematopoietic stem cells) comprising a) an inhibitory moiety comprising the amino acid sequence of C34 (SEQ ID NO: 8) or a function portion thereof, and b) a membrane domain that tethers the molecule to the membrane or facilitates the tethering of the molecule to the membrane. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, there is provided a method of treating an individual infected with HIV, comprising administering to the individual an effective amount of engineered immune cells (e.g., T cells, NK cells) comprising a) a binding moiety, wherein the binding moiety specifically binds to a T cell surface antigen and prevents the binding of the T cell surface antigen to its cognitive ligand on HIV, wherein the T cell surface antigen is selected from the group consisting of CCR5, CD4, and CXCR4, and b) a membrane domain that tethers the molecule to the membrane or facilitates the tethering of the molecule to the membrane. In some embodiments, the surface molecule does not comprise an intracellular signaling domain.

In some embodiments, there is provided a method of treating an individual infected with HIV, comprising administering to the individual an effective amount of engineered immune cells (e.g., T cells, NK cells) comprising a) a binding moiety, wherein the binding moiety specifically binds to CCR5, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, there is provided a method of treating an individual infected with HIV, comprising administering to the individual an effective amount of engineered immune cells (e.g., T cells, NK cells) comprising a) a binding moiety, wherein the binding moiety specifically binds to CCR5, and b) a membrane domain comprising a GPI attachment signal sequence (e.g., SEQ ID NO: 149), wherein the surface molecule does not comprise an intracellular signaling domain. In some embodiments, the binding moiety is a scFv comprising the amino acid sequence of SEQ ID NO: 1, 2, or 3, or a variant comprising at least about 80% sequence identity.

In some embodiments, there is provided a method of treating an individual infected with HIV, comprising administering to the individual an effective amount of engineered immune cells (e.g., T cells, NK cells) comprising a) a binding moiety, wherein the binding moiety specifically binds to CD4, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, there is provided a method of treating an individual infected with HIV, comprising administering to the individual an effective amount of engineered immune cells (e.g., T cells, NK cells) comprising a) a binding moiety, wherein the binding moiety specifically binds to CD4, and b) a membrane domain comprising a GPI attachment signal sequence (e.g., SEQ ID NO: 149), wherein the surface molecule does not comprise an intracellular signaling domain. In some embodiments, the binding moiety is a scFv comprising the amino acid sequence of SEQ ID NO: 4, 5, or 6, or a variant comprising at least about 80% sequence identity.

In some embodiments, there is provided a method of treating an individual infected with HIV, comprising administering to the individual an effective amount of engineered immune cells (e.g., T cells, NK cells) comprising a) a binding moiety, wherein the binding moiety specifically binds to CXCR4, and b) a membrane domain that tethers the molecule to the membrane, wherein the membrane domain is derived from or is a transmembrane domain from CD4, CD8, CD28, 4-1BB, or PD-1, wherein the surface molecule does not comprise an intracellular signaling domain. In some embodiments, there is provided a method of treating an individual infected with HIV, comprising administering to the individual an effective amount of engineered immune cells (e.g., T cells, NK cells) comprising a) a binding moiety, wherein the binding moiety specifically binds to CXCR4, and b) a membrane domain comprising a GPI attachment signal sequence (e.g., SEQ ID NO: 149), wherein the surface molecule does not comprise an intracellular signaling domain.

In some embodiments, there is provided a method of treating an individual infected with HIV, comprising administering to the individual an effective amount of engineered immune cells (e.g., T cells, NK cells) comprising a) a binding moiety, wherein the binding moiety specifically binds to a HIV antigen and prevents the binding of the HIV antigen to the engineered cell, and b) a membrane domain that tethers the molecule to the membrane or facilitates the tethering of the molecule to the membrane. In some embodiments, the surface molecule does not comprise an intracellular signaling domain. In some embodiments, the binding moiety competitively with 10-1074, 10E8, or PGT121, or binds to the same epitope as that of 10-1074, 10E8, or PGT121. In some embodiments, the binding moiety is a scFv comprising the amino acid sequence of SEQ ID NO: 7, 61, or 62, or a variant comprising at least about 80% sequence identity.

In some embodiments, there is provided a method of treating an individual infected with HIV, comprising administering to the individual an effective amount of engineered immune cells (e.g., T cells, NK cells) comprising a) an inhibitory moiety that inhibits membrane fusion of HIV, and b) a membrane domain that tethers the molecule to the membrane or facilitates the tethering of the molecule to the membrane. In some embodiments, the inhibitory moiety is selected from the group consisting of C34, HP32, SC35EK, sifuvirtide, T20, and T2634, or functional portions thereof. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, there is provided a method of treating an individual infected with HIV, comprising administering to the individual an effective amount of engineered immune cells (e.g., T cells, NK cells) comprising a) an inhibitory moiety comprising the amino acid sequence of C34 (SEQ ID NO: 8) or a function portion thereof, and b) a membrane domain that tethers the molecule to the membrane or facilitates the tethering of the molecule to the membrane. In some embodiments, the membrane domain comprises a GPI attachment signal sequence (e.g., SEQ ID NO: 149). In some embodiments, the membrane domain is derived from or is a transmembrane domain from CD4, CD8, CD28, 4-1BB, or PD-1. In some embodiments, the surface molecule does not comprise an intracellular signaling domain.

In some embodiments, the engineered cells are autologous to the individual.

In some embodiments, the engineered cells are allogeneic to the individual.

In some embodiments, at least about 5% (such as at least about 5%, 6%, 7%, 8%. 9%, 10%, 12%, 14%, 16%, 18%, or 20%) of the T cells in the individual express the surface molecule after administration of the engineered cells. In some embodiments, the T cell are measured about 1, 2, 3, 4, 5, 6, or 7 days after administration of the engineered cells. In some embodiments, the T cell are measured about 1, 2, 3, 4, 5, 6, 7, 8, 9, or 10 weeks after administration of the engineered cells. In some embodiments, the T cell are measured about 1, 2, 3, 4, 5, or 6 months after administration of the engineered cells.

In some embodiments, at least about 20% (such as at least about 22%, 24%, 26%, 28%. 30%, 32%, 34%, 36%, 38%, or 40%) of the T cells in the individual are resistant to HIV infection after administration of the engineered cells. In some embodiments, the HIV resistance of the T cells are measured about 1, 2, 3, 4, 5, 6, or 7 days after administration of the engineered cells. In some embodiments, the HIV resistance of the T cells are measured about 1, 2, 3, 4, 5, 6, 7, 8, 9, or 10 weeks after administration of the engineered cells. In some embodiments, the HIV resistance of the T cells are measured about 1, 2, 3, 4, 5, or 6 months after administration of the engineered cells.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or environmentally or genetically prone to one or more of the diseases or disorders described herein (such as cancer or viral infection). In some embodiments, the individual has one or more risk factors associated with one or more diseases or disorders described herein.

In some embodiments, the engineered cell compositions of the invention are administered in combination with a second, third, or fourth agent (including, e.g., an antineoplastic agent, a growth inhibitory agent, a cytotoxic agent, or a chemotherapeutic agent) to treat diseases or disorders involving target antigen expression.

Viral infection treatments can be evaluated, for example, by viral load, duration of survival, quality of life, protein expression and/or activity.

In some embodiments, the pharmaceutical composition is administered at a dosage of at least about any of 10⁴, 10⁵, 10⁶, 10⁷, 10⁸, or 10⁹ cells/kg of body weight. In some embodiments, the pharmaceutical composition is administered at a dosage of any of about 10⁴ to about 10⁵, about 10⁵ to about 10⁶, about 10⁶ to about 10⁷, about 10⁷ to about 10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight.

In some embodiments, more than one type of engineered cells are administered, the different types of engineered cells may be administered to the individual simultaneously, such as in a single composition, or sequentially in any suitable order.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

In some embodiments, the methods of treating an infectious disease described herein further comprises administering to the individual a second anti-infectious disease agent. Suitable anti-infectious disease agents include, but are not limited to, anti-retroviral drugs, broad neutralization antibodies, toll-like receptor agonists, latency reactivation agents, CCR5 antagonists, immune stimulators (e.g., TLR ligands), vaccines, nucleoside reverse transcriptase inhibitors, nucleotide reverse transcriptase inhibitors, non-nucleoside reverse transcriptase inhibitors, HIV protease inhibitors, and fusion inhibitors. In some embodiments, the second anti-infectious agent is administered simultaneously with the engineered cells. In some embodiments, the second anti-infectious agent is administered sequentially with (e.g., prior to or after) the administration of the engineered cells.

### Articles of Manufacture and Kits

In some embodiments of the invention, there is provided an article of manufacture containing materials useful for the treatment of an infectious disease such as viral infection (for example infection by HIV). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an engineered cell of the invention. The label or package insert indicates that the composition is used for treating the particular condition. The label or package insert will further comprise instructions for administering the engineered cell composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In other embodiments, the package insert indicates that the composition is used for treating a target antigen-positive viral infection (for example infection by HIV).

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the invention include one or more containers comprising an engineered cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

Those skilled in the art will recognize that several embodiments are possible within the scope and spirit of this invention. The invention will now be described in greater detail by reference to the following non-limiting exemplary embodiments and examples. The following exemplary embodiments and examples further illustrate the invention but, of course, should not be construed as in any way limiting its scope.

### EXAMPLES

### Example 1. Surface-Anchored scFv Blocks Pseudovirus in Cell Pool

The GPI-scFv constructs were assayed for their capacity of blocking the pseudovirus using TZM-b1 cells. The GPI-anchored antibody constructs expressed on cells are listed in Table 3 below. The constructs also include a nucleic acid encoding GFP for detection.

TABLE 3
scFv and bnAb constructs used for the experiment

| scFv/bnAb constructs | Target |
|---|---|
| X5 | HIV gp120 |
| C1-13 | CCR5 |
| C1-814 | CCR5 |
| C1-816 | CCR5 |
| C2-05 | CD4 |
| C2-11 | CD4 |
| C2-13 | CD4 |
| C3-05 | CXCR4 |
| VRC01 | CD4 |
| 3BNC117 | CD4 |
| 10E8 | MPER |
| PGT121 | V3 |

### Preparation of GPI TZM-b1 Cells

GPI TZM-b1 cells were produced as described below. TZM-b1 cells were digested and centrifuged at 800 rpm for 5 min. Cell pellet was resuspended and counted. 2×10⁵ cells were transferred to a 24-well plate and the cell growth medium was refilled to 1 mL. After 24 hours, the supernatant was discarded, and 1 ml of complete medium containing lentivirus with GPI-scFvs constructs as listed in Table 3 was added to the cells for incubation. Cells were then digested, transferred into a 6-well plate and incubated. Green fluorescence under a fluorescence microscope was measured.

### Pseudovirus Infection

200 ul GPI TZM-b1 cell resuspension for each GPI-anchored antibody was plated in 96-well plates to test for resistance to HIV pseudovirus infection. Pseudovirus was added to each experimental group by replacing the supernatant in each well with 200 μl fresh complete medium with pseudovirus. The amount of virus added was related to the titer of pseudovirus. Positive control (PC) group was set up with blank TZM-b1 cells with pseudovirus infection. Negative control (NC) group was set up without pseudovirus infection. Cells were then incubated for 24 h, after which the supernatant was replaced with 20011.1 fresh complete medium, followed by another 24-hour incubation.

### Detection of Luminescence

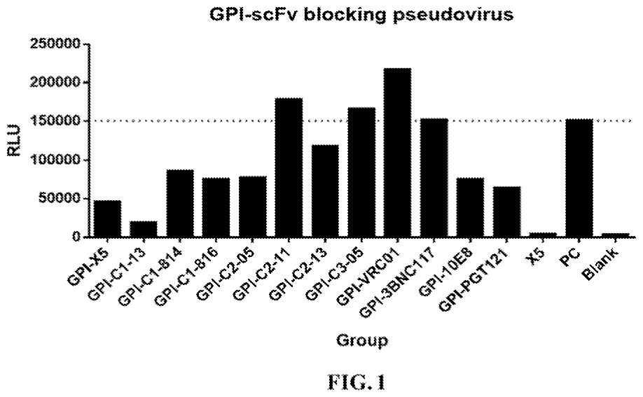

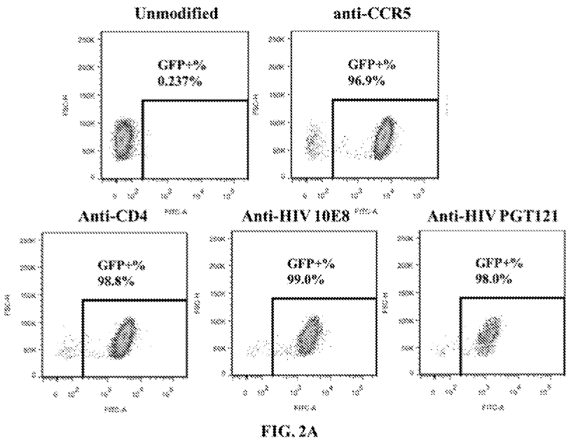

10011.1 of culture supernatant was aspirated. 10011.1 of One-Glo™ luciferase reagent was added to the wells and allowed to react for 15 minutes at room temperature. The average relative luminescence unit (RLU) was detected using a microplate reader. The RLU results are shown in FIG. 1.

FIG. 1 shows that the blocking effects of cells expressing GPI-anchored various antibodies. As shown, three GPI-anchored anti-CCR5 antibodies, i.e., GPI-C1-13, GPI-C1-814, and GPI-C1-816 provided protection against pseudovirus infection. Among those, GPI-C1-13 provided the highest level of protection against virus infection. GPI-anchored anti-CD4 antibodies, e.g., GPI-C2-05 and GPI-C2-13 also provided protection against pseudovirus. Various GPI-anchored HIV bnAbs (e.g., GPI-10E8, GPI-PGT121, GPI-X5) provided different levels of protection.

### Example 2. Cell-Level Herd Immunity to HIV Infection

Generation of Monoclonal GPI-TZM-b1 Cell Line that Express Surface GPI-scFV

GPI-TZM-b1 cells were produced as described in Example 1. The cells were then digested and re-plated in a 96-well plate using a serial dilution method for plating in 20011.1 culture medium. Cells were cultivated in the incubator continuously for 10 days. An inverted microscope was used to observe whether there was a single cell cluster in the wells. Single cell clusters were checked under a fluorescence microscope to confirm whether the clusters have green fluorescence. Single cell clusters with green fluorescence were selected and expanded, and subjected to flow cytometry to detect the purity of monoclonal cells. The results are shown in FIG. 2A, where the monoclonal TZM-b1 cell lines expressing surface GPI-scFVs or HIV bnAbs have high purity, ranging from 96.9% to 99.0%. These results suggest that highly homogenous HIV-resistant cells can be obtained through cell sorting.

### Pseudovirus Infection

200 ul GPI TZM-b1 cell resuspension for each GPI-anchored antibody was plated in 96-well plates to test for resistance to AD8 pseudovirus infection (Giese et al., Viruses. 2020 April; 12(4): 459). Pseudovirus was added to each experimental group by replacing the supernatant in each well with 200 μl fresh complete medium with pseudovirus. The amount of virus added was related to the titer of pseudovirus. Positive control (PC) group was set up with blank TZM-b1 cells with pseudovirus infection. Cells were then incubated for 24 h, after which the supernatant was replaced with 200 μl fresh complete medium, followed by another 24-hour incubation.

As shown in FIG. 2B, all tested GPI-anchored antibody prevented AD8 pseudovirus infection.

### Cell-Level Herd Immunity

Herd immunity generally refers to the resistance to the spread of a contagious disease within a population that results if a sufficiently high proportion of individuals are immune to the disease, especially through vaccination. We tested whether cell-level herd immunity can be achieved by mixing HIV-resistant cells with HIV-nonresistant cells.

Blank TZM-b1 cells and GPI TZM-b1 cells were mixed at 100%, 75%, 60%, 45%, 35%, 15% of GPI TZM-b1 cells. GPI TZM-b1 cells used include mono-GPI-X5 (i.e., monoclonal GPI TZM-b1 cells expressing anti-X5 scFv, also called mono-X5, similar abbreviations for cells described below), mono-GPI-CCR5 (C1-13), mono-GPI-CD4 (C2-13), mono-GPI-C34, mono-GPI-10E8, mono-GPI-PGT121, mono-GPI-1-18, mono-GPI-10-1074, mono-GPI-N6, mono-GPI-VRC07-523, mono-GPI-VRC07 and mono-GPI-PGT128. Culture medium was refilled to 200 ul. Cells were then incubated with pseudovirus for 24 hours. Positive group (PC) was set to be blank TZM-b1 cells with pseudovirus infection. 100% GPI TZM-b1 cells without virus infection were set up as negative control (NC) groups. Another negative control group of blank TZM-b1 cells without pseudovirus infection was also tested.

RLU of each group was detected as described in Example 1. The RLU of each group of GPI TZM-b1 cells without virus infection was basically equivalent to the RLU of blank TZM-b1 cells without pseudovirus infection. Therefore, the RLU of NC group was used as the background of all wells for calculation of blocking effect. Blocking effect (%) was calculated as 1-(RLU_{GPI-TZM-b1}-RLU_{NC})/(RLU_{PC}-RLU_{NC})*100%.

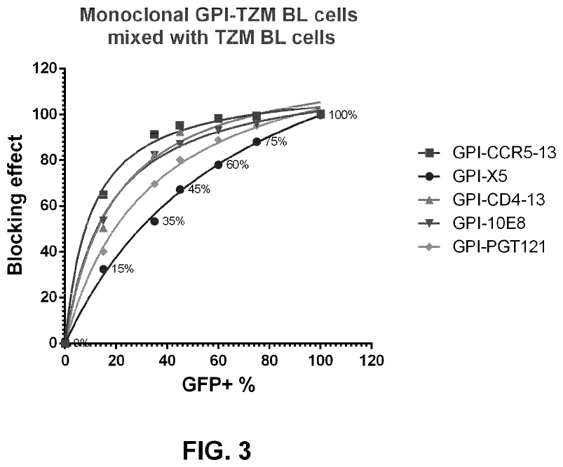

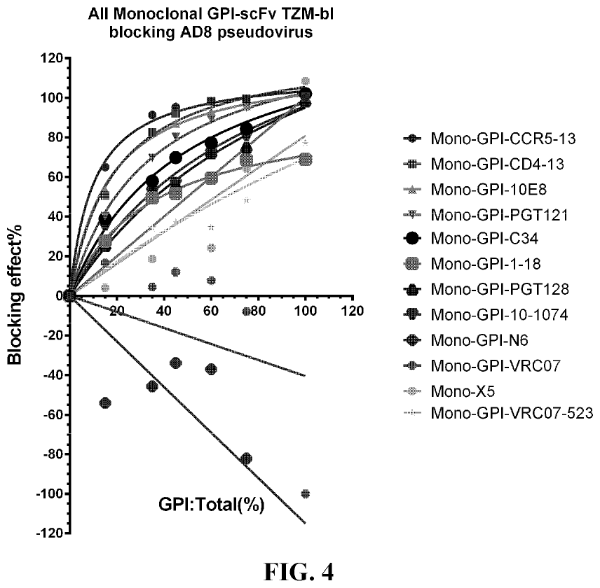

As shown in FIGS. 3 and 4, the percentage of cells expressing GPI-anchored HIV-resistant protein (e.g., antibody moieties specifically binding to CCR5, CD4, HIV antigen, or inhibitory moiety such as C34) was denoted as X-axis and the percentage of cells protected against infection was denoted as Y-axis. The mono-GPI-X5 has a close to linear blocking effect profile (FIG. 3), suggesting that a minimum herd immunity. On the other hand, mono-GPI-N6 shows enhancement of infection at high population percentage (FIG. 4). Strikingly, GPI-anchored anti-CCR5 (GPI-CCR5-13, i.e., GPI-C1-13) scFv expressing cells exhibited cell-level herd immunity, conferring more than 50% blocking at only 15% of the cell population, and approximately 90% protection at 50% population. GPI-C2-13 (i.e., GPI-CD4-13), GPI-10E8, and GPI-PGT121 also exhibited herd immunity to different level. FIG. 4 confirmed the striking herd immunity phenomenon exhibited by mono-GPI-CCR5. FIG. 4 also shows that mono-GPI-C34, mono-GPI-CD4 (C2-13), mono-GPI-10-1074, mono-GPI-10E8 and mono-GPI-PGT121 also exhibited herd immunity.

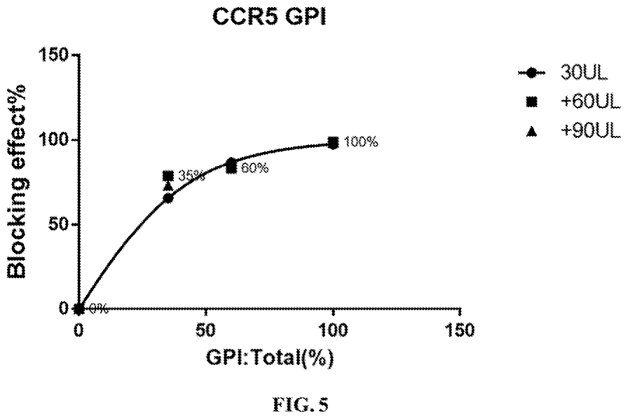

To test whether herd immunity persists in high dose of virus, mono-GPI-CCR5 (i.e., GPI-C1-13) was incubated with different doses of pseudovirus. As shown in FIG. 5, higher doses of virus (60 μl, 80 μl, or 90 μl of virus) did not affect the herd immunity phenomenon for mono-GPI-CCR5. On the other hand, higher dose of virus affected that resistance of mono-GPI-10E8 and mono-GPI-10-1074 (data not shown).

To our knowledge, our findings for the first time discovered a cell-level herd immunity phenomenon. By taking advantage of this effect, we can devise methods of treatment that could largely benefit diseases associated with pathogen (e.g., virus) infection. Specifically, we have provided methods to generate high-purity monoclonal cells expressing GPI-anchored surface molecules, and convincing evidence that these cells (such as cells expressing GPI-anchored anti-CCR5 scFv, GPI-anchored C34, GPI-anchored anti-CD4 scFv, or various GPI-anchored anti-HIV scFv) confer herd immunity.

### Example 3. Blocking Effects of Cells Expressing Various GPI-Anchored Anti-CCR5 Constructs

Construction of GPI-scFvs that Target CCR5

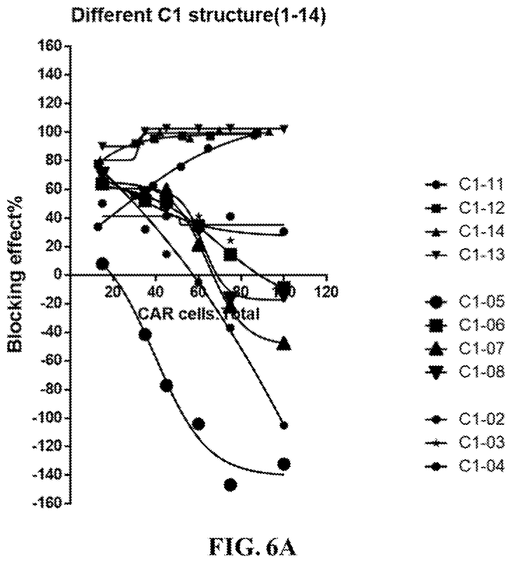

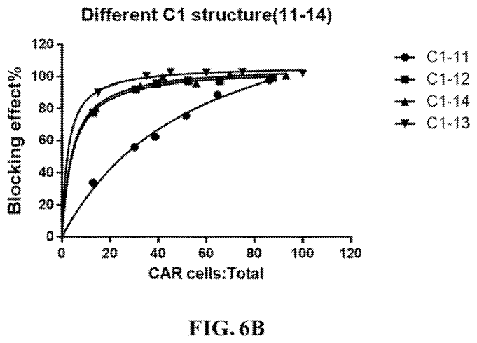

Various GPI-scFvs exhibited in FIG. 6A have from N-terminus to C-terminus: CD8a signal peptide (SEQ ID NO: 148)—[scFv—linker sequence—GPI attachment sequence] (any of SEQ ID NO: 137-147)—GFP (SEQ ID NO: 153).

### Preparation of GPI TZM-b1 Cells

GPI TZM-b1 cells were produced as described below. TZM-b1 cells were digested and centrifuged at 800 rpm for 5 min. Cell pellet was resuspended and counted. 2×10⁵ cells were transferred to a 24-well plate and the cell growth medium was refilled to 1 mL. After 24 hours, the supernatant was discarded, and 1 ml of complete medium containing lentivirus with GPI-scFvs (constructs described above) was added to the cells for incubation. Cells were then digested, transferred into a 6-well plate and incubated. Green fluorescence under a fluorescence microscope was measured.

### Cell-Level Herd Immunity

Cell-level herd immunity of these GPI TZM-b1 cells was measured as described in Example 2. As shown in FIGS. 6A-6B, cells transduced with GPI-anchored C1-11, C1-12, C1-13, or C1-14 exhibited excellent blocking effects against pseudovirus. Among them, cells transduced with GPI-anchored C1-11, C1-12, or C1-13 exhibited cell-level herd immunity.

### Example 4

### Construction of CAR-scFv Constructs

Exemplary CAR-scFv constructs (CAR-CD4-11 (i.e., CAR-C2-11), CAR-CD4-13 (i.e., CAR-C2-13), CAR-CCR5-13 (i.e., CAR-598) (C1-13), CAR-600 (10E8)) have from N-terminus to C-terminus: CD8a signal peptide (SEQ ID NO: 148)—scFv—CD8 transmembrane domain sequence (SEQ ID NO: 166). Exemplary CAR-scFv constructs have amino acid sequences set forth in SEQ ID NO: 167-171.

The CAR-scFv constructs do not have an intracellular signaling domain. A control construct that has the CAR but not the scFv was also constructed.

### Preparation of Primary T Cells Expressing CAR-scFv Construct

CD4+ T cells were isolated from PMBC obtained from healthy donors. These CD4+ T cells were then transduced with CAR-scFv constructs or the control CAR construct prepared as above. CD4+ T cells expressing the CAR-scFv construct were enriched incubating cells with an anti-scFv antibody and sorting out cells that are positive with the anti-scFv antibody.

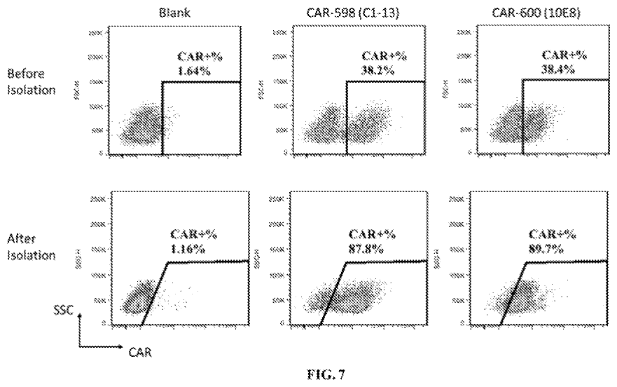

As shown in FIG. 7, a purity of about 90% CAR+ cells was achieved after the enrichment.

### Cell-Level Herd Immunity

Cell-level herd immunity of these primary CD4+ T cells expressing a CAR-scFv construct was evaluated. Specifically, blank PBMC and PBMC expressing CAR-scFv were mixed at a ratio of 0:4, 1:3, 2:2, 3:1 and 4:0. Cells were then incubated with pseudovirus for 24 hours. Positive group (PC) was set to be blank CD4+ primary T cells with pseudovirus infection. 100% CD4+ primary T cells expressing a CAR-scFv construct without virus infection were set up as negative control (NC) groups. Another negative control group of blank CD4+ primary T cells without pseudovirus infection was also tested.

RLU of each group was detected as described in Example 1. The RLU of each group of CD4+ primary T cells expressing a CAR-scFv construct without virus infection was basically equivalent to the RLU of blank CD4+ primary T cells without pseudovirus infection. Therefore, the RLU of NC group was used as the background of all wells for calculation of blocking effect. Blocking effect (%) was calculated as 1-(RLU_{CAR-cD4T}-RLU_{NC})/(RLU_{PC}-RLU_{NC})*100%.

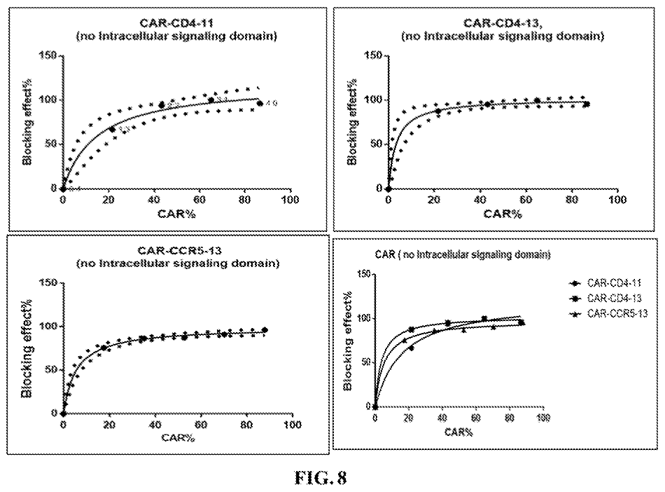

As shown in FIG. 8, primary CD4+ T cells expressing CAR-CD4-11, CAR-CD4-13, or CAR-CCR5-13 all exhibited excellent blocking effect and cell-level herd immunity against the HIV pseudovirus.

### Example 5. Cell-Level Herd Immunity Against HIV Exhibited by Cells Expressing Anti-CCR5 Antibody

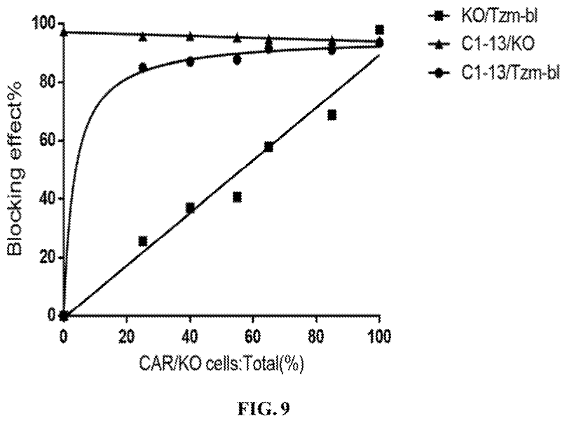

To further evaluate the cell-level herd immunity effect against HIV exhibited by cells expressing an anti-CCR5 antibody, Tzm-b1 cells expressing GPI-C1-13 (i.e., C1-13 as shown in FIG. 9) were prepared as described in Example 3. CCR5 KO Tzm-b1 cells (KO as shown in FIG. 9) were also prepared by introducing to Tzm-b1 cells a single guide RNA that target CCR5.

Two types of cells (Type A and Type B cells) were mixed at 0%, 25%, 40%, 55%, 65%, 85% or 100% of Type B cells as illustrated in Table 4 below. Blocking effect (%) was calculated as described above.

TABLE 4

| | Type A | | Represented in |
|---|---|---|---|
| | cells | Type B cells | FIG. 9 |
| Test #1 | Tzm-bl | CCR5 KO TZM-bl | Line connected by |
| | | cells | triangles |
| Test #2 | CCR5 KO | Tzm-bl cells | Line connected by |
| | TZM-bl | expressing GPI-C1-13 | squares |
| | cells | | |
| Test #3 | Tzm-bl | Tzm-bl cells | Line connected by |
| | | expressing GPI-C1-13 | dots |

As shown in FIG. 9, when the Tzm-b1 cells expressing GPI-C1-13 were mixed with CCR5KO TZM-b1 cells, the blocking effect was always close to 100% regardless of the ratio of the two cells, suggesting that the anti-CCR5 scFv on TZM-b1 cells and CCR5 KO on TZM-b1 cells can both protect cells from virus infection. When TZM-b1 cells were mixed with CCR5 KO TZM-b1 cells, the blocking effect increased in a linear manner when the proportion of CCR5 KO TZM-b1 cells was increased. When the Tzm-b1 cells expressing GPI-C1-13 and Tzm-b1 cells were mixed and when the proportion of the Tzm-b1 cells expressing GPI-C1-13 increased, the blocking effect increased in a non-linear manner, again demonstrating the cell-level herd immunity against the HIV pseudovirus.

SEQUENCE TABLE
Sequences of exemplary constructs according
to embodiments of the invention:

| SEQ | | |
|---|---|---|
| ID | | |
| NO. | Description | Nucleotide or Amino Acid Sequence |
| 1. | C1-13 scFv | QVQLVESGGGVVQPGRSLRLSCAASGFTLSGYGMHWVRQA |
| | | PGKGLEWVSLISYDGSNKYYADSVKGRFTISRDDSKNTLYL |
| | | RMNSLRAEDTAVYYCARGRNDFWSGYYTAGMDVWGQGT |
| | | TVTVSSGGGGSGGGGSGGGGSDIQMTQSPSSLSASVGDRVTI |
| | | TCQASQGIRKYLNWYQQKPGKVPKLLIYDASNLETGVPSRF |
| | | SGSGSGTDFTFAISSLQPEDTATYYCQQYDDFPFTFGQGTRLE |
| | | IKR |
| 2. | C1-814 scFv | EVQLVESGGGLVQPKGSLKLSCAASGFTFNTYAMNWVRQA |
| | | PGKGLEWVARIRNKSNNYATYYAASVKDRFTISRDDSQSML |
| | | YLQMNNLKTEDTAMYYCVSLGEFAYWGQGTLVTVSAGGG |
| | | GSGGGGSGGGGSEIVLTQSPTTMAASPGEKVTITCSATSSINS |
| | | NYLHWYQQRPGFSPKLLIYRTSNLASGVPARFSGSGSGTSYS |
| | | LTIGTMEAEDVATYYCQQGSTLPFTFGSGTKLEIK |
| 3 | C1-816 scFv | EVQLVESGGGLVQPKGSLKLSCAASGFTFNTYAMNWVRQA |
| | | PGKGLEWIARIRNKSNNYATYYAASVKDRFTISRDDSQSML |
| | | YLQMNNLKTEDTAMYYCVSLGEFAYWGQGTLVTVSAGGG |
| | | GSGGGGSGGGGSEIVLTQSPTTMAASPGEKVTITCSATSSINS |
| | | NYLHWYQQKPGFSPKLLIYRTSNLASGVPPRFSGSGSGTSYS |
| | | LTIGTMEAEDVATYYCQQGSTLPFTFGSGTKLEIK |
| 4 | C2-05 scFv | EVQLVESGGGLVQPGRSLRLSCAASGFTFSNYGMAWVRQA |
| | | PGKGLEWVATISYDGSITYYRDSVKGRFTISRDNSKNTLYLQ |
| | | MNSLRAEDTATYYCTREEQYSSWYFDFWGQGILVTVSSGG |
| | | GGSGGGGSGGGGSDIQLTQSPSSLSASVGDRVTITCRASQSV |
| | | SISSHDLMQWYQQKPGKAPKLLIYDAFNLASGVPSRFSGSGS |
| | | GTDFTLTISSLQPEDFATYYCQQSKDDPYTFGQGTKLEIK |
| 5. | C2-11 scFv | QVQLQQSGPEVVKPGASVKMSCKASGYTFTSYVIHWVRQK |
| | | PGQGLDWIGYINPYNDGTDYDEKFKGKATLTSDTSTSTAYM |
| | | ELSSLRSEDTAVYYCAREKDNYATGAWFAYWGQGTLVTVS |
| | | SAGGGGSGGGGSGGGGSDIVMTQSPDSLAVSLGERVTMNC |
| | | KSSQSLLYSTNQKNYLAWYQQKPGQSPKLLIYWASTRESGV |
| | | PDRFSGSGSGTDFTLTISSVQAEDVAVYYCQQYYSYRTFGGG |
| | | TKLEIKR |
| 6. | C2-13 scFv | QVQLQQWGAGLLKPSETLSLTCAVYGGSFSGYYWSWIRQPP |
| | | GKGLEWIGEINHSGSTNYNPSLKSRVTISVDTSKNQFSLKLSS |
| | | VTAADTAVYYCARVINWFDPWGQGTLVTGGGGSGGGGSG |
| | | GGGSDIQMTQSPSSVSASVGDRVTITCRASQDISSWLAWYQ |
| | | HKPGKAPKLLIYAASSLQSGVPSRFSGSGSGTDFTLTISSLQPE |
| | | DFATYYCQQANSFPYTFGQGTKLEIK |
| 7. | 10-1074 scFv | QVQLQESGPGLVKPSETLSVTCSVSGDSMNNYYWTWIRQSP |
| | | GKGLEWIGYISDRESATYNPSLNSRVVISRDTSKNQLSLKLNS |
| | | VTPADTAVYYCATARRGQRIYGVVSFGEFFYYYSMDVWGK |
| | | GTTVTVSSGGGGSGGGGSGGGGSSYVRPLSVALGETARISC |
| | | GRQALGSRAVQWYQHRPGQAPILLIYNNQDRPSGIPERFSGT |
| | | PDINFGTRATLTISGVEAGDEADYYCHMWDSRSGFSWSFGG |
| | | ATRLTVL |
| 8. | C34 | TMKHLWFFLLLVAAPRWVLSSWMEWDREINNYTSLIHSLIE |
| | | ESQNQQEKNEQELL |
| 9. | C1-13/C1-14 | GYGMH |
| | HC-CDR1 | |
| 10. | C1-13/C1-14 | LISYDGSNKYYADSVKG |
| | HC-CDR2 | |
| 11. | C1-13/C1-14 | GRNDFWSGYYTAGMDV |
| | HC-CDR3 | |
| 12. | C1-13/C1-14 | QASQGIRKYLN |
| | LC-CDR1 | |
| 13. | C1-13/C1-14 | DASNLET |
| | LC-CDR2 | |
| 14. | C1-13/C1-14 | QQYDDFPFT |
| | LC-CDR3 | |
| 15. | C1-814/C1- | TYAMN |
| | 816 HC-CDR1 | |
| 16 | C1-814/C1- | RIRNKSNNYATYYAASVKD |
| | 816 HC-CDR2 | |
| 17. | C1-814/C1- | LGEFAY |
| | 816 HC-CDR3 | |
| 18. | C1-814/C1- | SATSSINSNYLH |
| | 816 LC-CDR1 | |
| 19. | C1-814/C1- | RTSNLAS |
| | 816 LC-CDR2 | |
| 20. | C1-814/C1- | QQGSTLPFT |
| | 816 LC-CDR3 | |
| 21 | C2-05 HC- | NYGMA |
| | CDR1 | |
| 22. | C2-05 HC- | TISYDGSITYYRDSVKG |
| | CDR2 | |
| 23 | C2-05 HC- | EEQYSSWYFDF |
| | CDR3 | |
| 24. | C2-05 LC- | RASQSVSISSHDLMQ |
| | CDR1 | |
| 25. | C2-05 LC- | DAFNLAS |
| | CDR2 | |
| 26. | C2-05 LC- | QQSKDDPYT |
| | CDR3 | |
| 27 | C2-11 HC- | SYVIH |
| | CDR1 | |
| 28. | C2-11 HC- | YINPYNDGTDYDEKFKG |
| | CDR2 | |
| 29. | C2-11 HC- | EKDNYATGAWFAY |
| | CDR3 | |
| 30. | C2-11 LC- | KSSQSLLYSTNQKNYLA |
| | CDR1 | |
| 31. | C2-11 LC- | WASTRES |
| | CDR2 | |
| 32. | C2-11 LC- | QQYYSYRT |
| | CDR3 | |
| 33. | C2-13 HC- | GYYWS |
| | CDR1 | |
| 34. | C2-13 HC- | EINHSGSTNYNPSLKS |
| | CDR2 | |
| 35. | C2-13 HC- | VINWFDP |
| | CDR3 | |
| 36. | C2-13 LC- | RASQDISSWLA |
| | CDR1 | |
| 37. | C2-13 LC- | AASSLQS |
| | CDR2 | |
| 38. | C2-13 LC- | QQANSFPYT |
| | CDR3 | |
| 39 | 10-1074 HC- | NYYWT |
| | CDR1 | |
| 40. | 10-1074 HC- | YISDRESATYNPSLNS |
| | CDR2 | |
| 41. | 10-1074 HC- | ARRGQRIYGVVSFGEFFYYYSMDV |
| | CDR3 | |
| 42. | 10-1074 LC- | GRQALGSRAVQ |
| | CDR1 | |
| 43. | 10-1074 LC- | NNQDRPS |
| | CDR2 | |
| 44. | 10-1074 LC- | HMWDSRSGFSWS |
| | CDR3 | |
| 45. | Exemplary | GGGGSGGGGSGGGGS |
| | linker | |
| 46. | Exemplary | (GGGGS)n, n is between 1 and 8. |
| | linker | |
| 47. | Exemplary | (GGGGS)6 |
| | linker | |
| 48. | Exemplary | (GSTSGSGKPGSGEGS)n, |
| | linker | n is between 1 and 3. |
| 49. | Exemplary | (G)n |
| | linker | |
| 50. | Exemplary | (GS)n |
| | linker | |
| 51. | Exemplary | (GSGGS)n |
| | linker | |
| 52. | Exemplary | (GGGS)n |
| | linker | |
| 53. | Exemplary | EPKSSDKTHTSPPSP |
| | linker | |
| 54. | Exemplary | EPKSSDKGHGGPPGP |
| | linker | |
| 55. | Exemplary | ERKSSVESPPSP |
| | linker | |
| 56. | Exemplary | ERKSGVEGPPGP |
| | linker | |
| 57. | Exemplary | ESKYGPPSPPSP |
| | linker | |
| 58. | Exemplary | ESKYGPPGPPGP |
| | linker | |
| 59. | Exemplary | GGGGSGGGGSGS |
| | linker | |
| 60. | Exemplary | GGGGSGGGS |
| | linker | |
| 61. | 10E8 | EVQLVESGGGLVKPGGSLRLSCSASGFDFDNAWMTWVRQP |
| | | PGKGLEWVGRITGPGEGWSVDYAAPVEGRFTISRLNSINFLY |
| | | LEMNNLRMEDSGLYFCARTGKYYDFWSGYPPGEEYFQDWG |
| | | RGTLVTVSSGGGGSGGGGSGGGGSSYELTQETGVSVALGRT |
| | | VTITCRGDSLRSHYASWYQKKPGQAPILLFYGKNNRPSGVP |
| | | DRFSGASGNRASLTISGAQAEDDAEYYCSSRDKSGSRLSVFG |
| | | GGTKLTVL |
| 62. | PGT-121 | QMQLQESGPGLVKPSETLSLTCSVSGASISDSYWSWIRRSPG |
| | | KGLEWIGYVHKSGDTNYSPSLKSRVNLSLDTSKNQVSLSLV |
| | | AATAADSGKYYCARTLHGRRIYGIVAFNEWFTYFYMDVWG |
| | | NGTQVTVSSGGGGSGGGGSGGGGSSDISVAPGETARISCGEK |
| | | SLGSRAVQWYQHRAGQAPSLIIYNNQDRPSGIPERFSGSPDSP |
| | | FGTTATLTITSVEAGDEADYYCHIWDSRVPTKWVFGGGTTL |
| | | TVL |
| 63. | 10E8 HC- | NAWMT |
| | CDR1 | |
| 64. | 10E8 HC- | RITGPGEGWSVDYAAPVEG |
| | CDR2 | |
| 65. | 10E8 HC- | TGKYYDFWSGYPPGEEYFQD |
| | CDR3 | |
| 66. | 10E8 LC- | RGDSLRSHYAS |
| | CDR1 | |
| 67. | 10E8 LC- | GKNNRPS |
| | CDR2 | |
| 68 | 10E8 LC- | SSRDKSGSRLSV |
| | CDR3 | |
| 69. | PGT-121 HC- | DSYWS |
| | CDR1 | |
| 70. | PGT-121 HC- | YVHKSGDTNYSPSLKS |
| | CDR2 | |
| 71. | PGT-121 HC- | TLHGRRIYGIVAFNEWFTYFYMDV |
| | CDR3 | |
| 72. | PGT-121 LC- | GEKSLGSRAVQ |
| | CDR1 | |
| 73. | PGT-121 LC- | NNQDRPS |
| | CDR2 | |
| 74 | PGT-121 LC- | HIWDSRVPTKWV |
| | CDR3 | |
| 75. | C1-13/C1-14 | QVQLVESGGGVVQPGRSLRLSCAASGFTLSGYGMHWVRQA |
| | VH | PGKGLEWVSLISYDGSNKYYADSVKGRFTISRDDSKNTLYL |
| | | RMNSLRAEDTAVYYCARGRNDFWSGYYTAGMDVWGQGT |
| | | TVTVSS |
| 76. | C1-13/C1-14 | DIQMTQSPSSLSASVGDRVTITCQASQGIRKYLNWYQQKPG |
| | VL | KVPKLLIYDASNLETGVPSRFSGSGSGTDFTFAISSLQPEDTA |
| | | TYYCQQYDDFPFTFGQGTRLEIKR |
| 77. | C1-814 VH | EVQLVESGGGLVQPKGSLKLSCAASGFTFNTYAMNWVRQA |
| | | PGKGLEWVARIRNKSNNYATYYAASVKDRFTISRDDSQSML |
| | | YLQMNNLKTEDTAMYYCVSLGEFAYWGQGTLVTVSA |
| 78. | C1-814 VL | EIVLTQSPTTMAASPGEKVTITCSATSSINSNYLHWYQQRPGF |
| | | SPKLLIYRTSNLASGVPARFSGSGSGTSYSLTIGTMEAEDVAT |
| | | YYCQQGSTLPFTFGSGTKLEIK |
| 79 | C1-816 VH | EVQLVESGGGLVQPKGSLKLSCAASGFTFNTYAMNWVRQA |
| | | PGKGLEWIARIRNKSNNYATYYAASVKDRFTISRDDSQSML |
| | | YLQMNNLKTEDTAMYYCVSLGEFAYWGQGTLVTVSA |
| 80. | C1-816 VL | EIVLTQSPTTMAASPGEKVTITCSATSSINSNYLHWYQQKPGF |
| | | SPKLLIYRTSNLASGVPPRFSGSGSGTSYSLTIGTMEAEDVAT |
| | | YYCQQGSTLPFTFGSGTKLEIK |
| 81 | C2-05 VH | EVQLVESGGGLVQPGRSLRLSCAASGFTFSNYGMAWVRQA |
| | | PGKGLEWVATISYDGSITYYRDSVKGRFTISRDNSKNTLYLQ |
| | | MNSLRAEDTATYYCTREEQYSSWYFDFWGQGILVTVSS |
| 82 | C2-05 VL | DIQLTQSPSSLSASVGDRVTITCRASQSVSISSHDLMQWYQQ |
| | | KPGKAPKLLIYDAFNLASGVPSRFSGSGSGTDFTLTISSLQPE |
| | | DFATYYCQQSKDDPYTFGQGTKLEIK |
| 83. | C2-11 VH | QVQLQQSGPEVVKPGASVKMSCKASGYTFTSYVIHWVRQK |
| | | PGQGLDWIGYINPYNDGTDYDEKFKGKATLTSDTSTSTAYM |
| | | ELSSLRSEDTAVYYCAREKDNYATGAWFAYWGQGTLVTVS |
| | | S |
| 84. | C2-11 VL | DIVMTQSPDSLAVSLGERVTMNCKSSQSLLYSTNQKNYLAW |
| | | YQQKPGQSPKLLIYWASTRESGVPDRFSGSGSGTDFTLTISSV |
| | | QAEDVAVYYCQQYYSYRTFGGGTKLEIKR |
| 85. | C2-13 VH | QVQLQQWGAGLLKPSETLSLTCAVYGGSFSGYYWSWIRQPP |
| | | GKGLEWIGEINHSGSTNYNPSLKSRVTISVDTSKNQFSLKLSS |
| | | VTAADTAVYYCARVINWFDPWGQGTLVT |
| 86. | C2-13 VL | DIQMTQSPSSVSASVGDRVTITCRASQDISSWLAWYQHKPG |
| | | KAPKLLIYAASSLQSGVPSRFSGSGSGTDFTLTISSLQPEDFAT |
| | | YYCQQANSFPYTFGQGTKLEIK |
| 87. | 10-1074 VH | QVQLQESGPGLVKPSETLSVTCSVSGDSMNNYYWTWIRQSP |
| | | GKGLEWIGYISDRESATYNPSLNSRVVISRDTSKNQLSLKLNS |
| | | VTPADTAVYYCATARRGQRIYGVVSFGEFFYYYSMDVWGK |
| | | GTTVTVSS |
| 88 | 10-1074 VL | SYVRPLSVALGETARISCGRQALGSRAVQWYQHRPGQAPIL |
| | | LIYNNQDRPSGIPERFSGTPDINFGTRATLTISGVEAGDEADY |
| | | YCHMWDSRSGFSWSFGGATRLTVL |
| 89. | 10E8 VH | EVQLVESGGGLVKPGGSLRLSCSASGFDFDNAWMTWVRQP |
| | | PGKGLEWVGRITGPGEGWSVDYAAPVEGRFTISRLNSINFLY |
| | | LEMNNLRMEDSGLYFCARTGKYYDFWSGYPPGEEYFQDWG |
| | | RGTLVTVSS |
| 90 | 10E8 VL | SYELTQETGVSVALGRTVTITCRGDSLRSHYASWYQKKPGQ |
| | | APILLFYGKNNRPSGVPDRFSGASGNRASLTISGAQAEDDAE |
| | | YYCSSRDKSGSRLSVFGGGTKLTVL |
| 91. | PGT-121 VH | QMQLQESGPGLVKPSETLSLTCSVSGASISDSYWSWIRRSPG |
| | | KGLEWIGYVHKSGDTNYSPSLKSRVNLSLDTSKNQVSLSLV |
| | | AATAADSGKYYCARTLHGRRIYGIVAFNEWFTYFYMDVWG |
| | | NGTQVT |
| 92. | PGT-121 VL | SDISVAPGETARISCGEKSLGSRAVQWYQHRAGQAPSLIIYN |
| | | NQDRPSGIPERFSGSPDSPFGTTATLTITSVEAGDEADYYCHI |
| | | WDSRVPTKWVFGGGTTLTVL |
| 93. | C1-02 HC- | TSGVSVG |
| | CDR1 | |
| 94. | C1-02 HC- | SINWNDDKCYSPSLKS |
| | CDR2 | |
| 95. | C1-02 HC- | DMPPHDSGPQSFDASDV |
| | CDR3 | |
| 96. | C1-02 LC- | SGDNLGDKYAC |
| | CDR1 | |
| 97. | C1-02 LC- | GDNKRPS |
| | CDR2 | |
| 98. | C1-02 LC- | QAWDTSTAV |
| | CDR3 | |
| 99. | C1-02 VH | QVTLKESGPTLVKPTQTLTLTCTLSGFSLSTSGVSVGWIRQPP |
| | | GKALEWLASINWNDDKCYSPSLKSRLTITKDTPKNQVVLAM |
| | | SNMDPADTATYSCALDMPPHDSGPQSFDASDVWGPGTMVT |
| | | VSS |
| 100. | C1-02 VL | SYELMQLPSVSVSPGQTASITCSGDNLGDKYACWYQQKPGR |
| | | SPVLVIYGDNKRPSGIPERFSGSNSGNTATLTISGTQAMDEAD |
| | | YYCQAWDTSTAVFGTGTKLTVL |
| 101. | C1-03/C1-04 | HDYWS |
| | HC-CDR1 | |
| 102. | C1-03/C1-04 | FIFFDGSTNYNPSLNG |
| | HC-CDR2 | |
| 103. | C1-03/C1-04 | LKGAWLLSEPPYFSSDGMDV |
| | HC-CDR3 | |
| 104. | C1-03/C1-04 | SGSSSDIGSNTVN |
| | LC-CDR1 | |
| 105. | C1-03/C1-04 | SNNQRPS |
| | LC-CDR2 | |
| 106. | C1-03/C1-04 | AAWDESLNGVV |
| | LC-CDR3 | |
| 107. | C1-03/C1-04 | QVQLQESGPGLVKPSETLSLTCTVSGGSIGHDYWSWIRQPPG |
| | VH | EGLEWIGFIFFDGSTNYNPSLNGRVTISLDTSKNQLSLRLTSV |
| | | TAADTAVYFCARLKGAWLLSEPPYFSSDGMDVWGQGTTVT |
| | | VPS |
| 108. | C1-03/C1-04 | NFMLTQPPSASGTPGQRVSISCSGSSSDIGSNTVNWYQQLPG |
| | VL | TAPKLLIYSNNQRPSGVPDRFSGFKSGTSASLVISGLQSEDEA |
| | | DYYCAAWDESLNGVVFGGGPR |
| 109. | C1-05/C1-06 | GYYWS |
| | HC-CDR1 | |
| 110. | C1-05/C1-06 | EINHRGSTTYNPSLDG |
| | HC-CDR2 | |
| 111. | C1-05/C1-06 | TVAGTSDY |
| | HC-CDR3 | |
| 112. | C1-05/C1-06 | KASRDVDDDVN |
| | LC-CDR1 | |
| 113. | C1-05/C1-06 | DATTLVP |
| | LC-CDR2 | |
| 114 | C1-05/C1-06 | LQHDNFPLT |
| | LC-CDR3 | |
| 115. | C1-05/C1-06 | QVQLQQWGAGLLKSWGTLSLTCAVSGASFSGYYWSWIRQP |
| | VH | PGKGLEWIGEINHRGSTTYNPSLDGRVTISLDTSTNQISLKLT |
| | | SMTAADTAVYYCARTVAGTSDYWGQGTLVTVSS |
| 116. | C1-05/C1-06 | KTTLTQSPAFMSATPGDKVSISCKASRDVDDDVNWYQQRPG |
| | VL | EAPIFIIEDATTLVPGISPRFSGSGYGTDFTLTINNIDSEDAAYY |
| | | FCLQHDNFPLTFGGGTKVEIK |
| 117. | C1-07/C1-08 | TTGEGVG |
| | HC-CDR1 | |
| 118. | C1-07/C1-08 | LIYWDDDKRYSPSLKS |
| | HC-CDR2 | |
| 119. | C1-07/C1-08 | EQYYYDTSGQPYYFDF |
| | HC-CDR3 | |
| 120. | C1-07/C1-08 | RASQDIRKNLN |
| | LC-CDR1 | |
| 121. | C1-07/C1-08 | DASDLET |
| | LC-CDR2 | |
| 122. | C1-07/C1-08 | QQSDYLPLT |
| | LC-CDR3 | |
| 123. | C1-07/C1-08 | QVTLKESGPTLVKPTQTLTLTCTFSGFSLRTTGEGVGWVRQP |
| | VH | PGKALEWLALIYWDDDKRYSPSLKSRLTITKDTSKKQVVLT |
| | | MTNVDPADTATYYCTHEQYYYDTSGQPYYFDFWGQGTLVT |
| | | VSS |
| 124. | C1-07/C1-08 | NIQVTQSPSSLSASVGDRVTMTCRASQDIRKNLNWYQQKPG |
| | VL | KAPKVLIYDASDLETGIPSRFSGSGSGTDFILTISSLQPEDIATY |
| | | YCQQSDYLPLTFGGGTKVDIK |
| 125. | C1-11/C1-12 | NYWIG |
| | HC-CDR1 | |
| 126 | C1-11/C1-12 | DIYPGGNYIRNNEKFKD |
| | HC-CDR2 | |
| 127. | C1-11/C1-12 | SFGSNYVFAWFTY |
| | HC-CDR3 | |
| 128. | C1-11/C1-12 | RSSQRLLSSYGHTYLH |
| | LC-CDR1 | |
| 129 | C1-11/C1-12 | EVSNRFS |
| | LC-CDR2 | |
| 130. | C1-11/C1-12 | SQSTHVPLT |
| | LC-CDR3 | |
| 131. | C1-11/C1-12 | EVQLVESGGGLVKPGGSLRLSCAASGYTFSNYWIGWVRQAP |
| | VH | GKGLEWIGDIYPGGNYIRNNEKFKDKTTLSADTSKNTAYLQ |
| | | MNSLKTEDTAVYYCGSSFGSNYVFAWFTYWGQGTLVTVSS |
| 132. | C1-11/C1-12 | DIVMTQSPLSLPVTPGEPASISCRSSQRLLSSYGHTYLHWYLQ |
| | VL | KPGQSPQLLIYEVSNRFSGVPDRFSGSGSGTDFTLKISRVEAE |
| | | DVGVYYCSQSTHVPLTFGQGTKVEIK |
| 133. | C1-11 scFv | EVQLVESGGGLVKPGGSLRLSCAASGYTFSNYWIGWVRQAP |
| | | GKGLEWIGDIYPGGNYIRNNEKFKDKTTLSADTSKNTAYLQ |
| | | MNSLKTEDTAVYYCGSSFGSNYVFAWFTYWGQGTLVTVSS |
| | | GGGGSGGGGSGGGGSDIVMTQSPLSLPVTPGEPASISCRSSQ |
| | | RLLSSYGHTYLHWYLQKPGQSPQLLIYEVSNRFSGVPDRFSG |
| | | SGSGTDFTLKISRVEAEDVGVYYCSQSTHVPLTFGQGTKVEI |
| | | K |
| 134. | C1-12 scFv | DIVMTQSPLSLPVTPGEPASISCRSSQRLLSSYGHTYLHWYLQ |
| | | KPGQSPQLLIYEVSNRFSGVPDRFSGSGSGTDFTLKISRVEAE |
| | | DVGVYYCSQSTHVPLTFGQGTKVEIKGGGGSGGGGSGGGGS |
| | | EVQLVESGGGLVKPGGSLRLSCAASGYTFSNYWIGWVRQAP |
| | | GKGLEWIGDIYPGGNYIRNNEKFKDKTTLSADTSKNTAYLQ |
| | | MNSLKTEDTAVYYCGSSFGSNYVFAWFTYWGQGTLVTVSS |
| 135. | C1-14 scFv | DIQMTQSPSSLSASVGDRVTITCQASQGIRKYLNWYQQKPG |
| | | KVPKLLIYDASNLETGVPSRFSGSGSGTDFTFAISSLQPEDTA |
| | | TYYCQQYDDFPFTFGQGTRLEIKRGGGGSGGGGSGGGGSQV |
| | | QLVESGGGVVQPGRSLRLSCAASGFTLSGYGMHWVRQAPG |
| | | KGLEWVSLISYDGSNKYYADSVKGRFTISRDDSKNTLYLRM |
| | | NSLRAEDTAVYYCARGRNDFWSGYYTAGMDVWGQGTTVT |
| | | VSS |
| 136. | GPI-10-1074 | QVQLQESGPGLVKPSETLSVTCSVSGDSMNNYYWTWIRQSP |
| | | GKGLEWIGYISDRESATYNPSLNSRVVISRDTSKNQLSLKLNS |
| | | VTPADTAVYYCATARRGQRIYGVVSFGEFFYYYSMDVWGK |
| | | GTTVTVSSGGGGSGGGGSGGGGSSYVRPLSVALGETARISC |
| | | GRQALGSRAVQWYQHRPGQAPILLIYNNQDRPSGIPERFSGT |
| | | PDINFGTRATLTISGVEAGDEADYYCHMWDSRSGFSWSFGG |
| | | ATRLTVLPGTPLGDTTHTSGYPYDVPDYALEGSGTTSGTTRL |
| | | LSGHTCFTLTGLLGTLVTMGLLT |
| 137. | GPI-C1-02 | SYELMQLPSVSVSPGQTASITCSGDNLGDKYACWYQQKPGR |
| | | SPVLVIYGDNKRPSGIPERFSGSNSGNTATLTISGTQAMDEAD |
| | | YYCQAWDTSTAVFGTGTKLTVLGGGGSGGGGSGGGGSQVT |
| | | LKESGPTLVKPTQTLTLTCTLSGFSLSTSGVSVGWIRQPPGKA |
| | | LEWLASINWNDDKCYSPSLKSRLTITKDTPKNQVVLAMSNM |
| | | DPADTATYSCALDMPPHDSGPQSFDASDVWGPGTMVTVSSP |
| | | GTPLGDTTHTSGYPYDVPDYALEGSGTTSGTTRLLSGHTCFT |
| | | LTGLLGTLVTMGLLT |
| 138. | GPI-C1-03 | QVQLQESGPGLVKPSETLSLTCTVSGGSIGHDYWSWIRQPPG |
| | | EGLEWIGFIFFDGSTNYNPSLNGRVTISLDTSKNQLSLRLTSV |
| | | TAADTAVYFCARLKGAWLLSEPPYFSSDGMDVWGQGTTVT |
| | | VPSGGGGSGGGGSGGGGSNFMLTQPPSASGTPGQRVSISCSG |
| | | SSSDIGSNTVNWYQQLPGTAPKLLIYSNNQRPSGVPDRFSGF |
| | | KSGTSASLVISGLQSEDEADYYCAAWDESLNGVVFGGGPRP |
| | | GTPLGDTTHTSGYPYDVPDYALEGSGTTSGTTRLLSGHTCFT |
| | | LTGLLGTLVTMGLLT |
| 139. | GPI-C1-04 | NFMLTQPPSASGTPGQRVSISCSGSSSDIGSNTVNWYQQLPG |
| | | TAPKLLIYSNNQRPSGVPDRFSGFKSGTSASLVISGLQSEDEA |
| | | DYYCAAWDESLNGVVFGGGPRGGGGSGGGGSGGGGSQVQ |
| | | LQESGPGLVKPSETLSLTCTVSGGSIGHDYWSWIRQPPGEGL |
| | | EWIGFIFFDGSTNYNPSLNGRVTISLDTSKNQLSLRLTSVTAA |
| | | DTAVYFCARLKGAWLLSEPPYFSSDGMDVWGQGTTVTVPS |
| | | PGTPLGDTTHTSGYPYDVPDYALEGSGTTSGTTRLLSGHTCF |
| | | TLTGLLGTLVTMGLLT |
| 140. | GPI-C1-05 | QVQLQQWGAGLLKSWGTLSLTCAVSGASFSGYYWSWIRQP |
| | | PGKGLEWIGEINHRGSTTYNPSLDGRVTISLDTSTNQISLKLT |
| | | SMTAADTAVYYCARTVAGTSDYWGQGTLVTVSSGSASAPT |
| | | GGGGSGGGGSGGGGSKTTLTQSPAFMSATPGDKVSISCKAS |
| | | RDVDDDVNWYQQRPGEAPIFIIEDATTLVPGISPRFSGSGYGT |
| | | DFTLTINNIDSEDAAYYFCLQHDNFPLTFGGGTKVEIKPGTPL |
| | | GDTTHTSGYPYDVPDYALEGSGTTSGTTRLLSGHTCFTLTGL |
| | | LGTLVTMGLLTGSGATNFSLLKQAGDVEENPGPMVSKGEEL |
| | | FTGVVPILVELDGDVNGHKFSVSGEGEGDATYGKLTLKFICT |
| | | TGKLPVPWPTLVTTLTYGVQCFSRYPDHMKQHDFFKSAMPE |
| | | GYVQERTIFFKDDGNYKTRAEVKFEGDTLVNRIELKGIDFKE |
| | | DGNILGHKLEYNYNSHNVYIMADKQKNGIKVNFKIRHNIED |
| | | GSVQLADHYQQNTPIGDGPVLLPDNHYLSTQSALSKDPNEK |
| | | RDHMVLLEFVTAAGITLGMDELYKSG |
| 141. | GPI-C1-06 | KTTLTQSPAFMSATPGDKVSISCKASRDVDDDVNWYQQRPG |
| | | EAPIFIIEDATTLVPGISPRFSGSGYGTDFTLTINNIDSEDAAYY |
| | | FCLQHDNFPLTFGGGTKVEIKGGGGSGGGGSGGGGSQVQLQ |
| | | QWGAGLLKSWGTLSLTCAVSGASFSGYYWSWIRQPPGKGL |
| | | EWIGEINHRGSTTYNPSLDGRVTISLDTSTNQISLKLTSMTAA |
| | | DTAVYYCARTVAGTSDYWGQGTLVTVSSGSASAPTPGTPLG |
| | | DTTHTSGYPYDVPDYALEGSGTTSGTTRLLSGHTCFTLTGLL |
| | | GTLVTMGLLT |
| 142. | GPI-C1-07 | QVTLKESGPTLVKPTQTLTLTCTFSGFSLRTTGEGVGWVRQP |
| | | PGKALEWLALIYWDDDKRYSPSLKSRLTITKDTSKKQVVLT |
| | | MTNVDPADTATYYCTHEQYYYDTSGQPYYFDFWGQGTLVT |
| | | VSSGGGGSGGGGSGGGGSNIQVTQSPSSLSASVGDRVTMTC |
| | | RASQDIRKNLNWYQQKPGKAPKVLIYDASDLETGIPSRFSGS |
| | | GSGTDFILTISSLQPEDIATYYCQQSDYLPLTFGGGTKVDIKP |
| | | GTPLGDTTHTSGYPYDVPDYALEGSGTTSGTTRLLSGHTCFT |
| | | LTGLLGTLVTMGLLT |
| 143. | GPI-C1-08 | NIQVTQSPSSLSASVGDRVTMTCRASQDIRKNLNWYQQKPG |
| | | KAPKVLIYDASDLETGIPSRFSGSGSGTDFILTISSLQPEDIATY |
| | | YCQQSDYLPLTFGGGTKVDIKGGGGSGGGGSGGGGSQVTL |
| | | KESGPTLVKPTQTLTLTCTFSGFSLRTTGEGVGWVRQPPGKA |
| | | LEWLALIYWDDDKRYSPSLKSRLTITKDTSKKQVVLTMTNV |
| | | DPADTATYYCTHEQYYYDTSGQPYYFDFWGQGTLVTVSSP |
| | | GTPLGDTTHTSGYPYDVPDYALEGSGTTSGTTRLLSGHTCFT |
| | | LTGLLGTLVTMGLLT |
| 144. | GPI-C1-11 | EVQLVESGGGLVKPGGSLRLSCAASGYTFSNYWIGWVRQAP |
| | | GKGLEWIGDIYPGGNYIRNNEKFKDKTTLSADTSKNTAYLQ |
| | | MNSLKTEDTAVYYCGSSFGSNYVFAWFTYWGQGTLVTVSS |
| | | GGGGSGGGGSGGGGSDIVMTQSPLSLPVTPGEPASISCRSSQ |
| | | RLLSSYGHTYLHWYLQKPGQSPQLLIYEVSNRFSGVPDRFSG |
| | | SGSGTDFTLKISRVEAEDVGVYYCSQSTHVPLTFGQGTKVEI |
| | | KPGTPLGDTTHTSGYPYDVPDYALEGSGTTSGTTRLLSGHTC |
| | | FTLTGLLGTLVTMGLLT |
| 145. | GPI-C1-12 | DIVMTQSPLSLPVTPGEPASISCRSSQRLLSSYGHTYLHWYLQ |
| | | KPGQSPQLLIYEVSNRFSGVPDRFSGSGSGTDFTLKISRVEAE |
| | | DVGVYYCSQSTHVPLTFGQGTKVEIKGGGGSGGGGSGGGGS |
| | | EVQLVESGGGLVKPGGSLRLSCAASGYTFSNYWIGWVRQAP |
| | | GKGLEWIGDIYPGGNYIRNNEKFKDKTTLSADTSKNTAYLQ |
| | | MNSLKTEDTAVYYCGSSFGSNYVFAWFTYWGQGTLVTVSS |
| | | PGTPLGDTTHTSGYPYDVPDYALEGSGTTSGTTRLLSGHTCF |
| | | TLTGLLGTLVTMGLLT |
| 146. | GPI-C1-13 | QVQLVESGGGVVQPGRSLRLSCAASGFTLSGYGMHWVRQA |
| | | PGKGLEWVSLISYDGSNKYYADSVKGRFTISRDDSKNTLYL |
| | | RMNSLRAEDTAVYYCARGRNDFWSGYYTAGMDVWGQGT |
| | | TVTVSSGGGGSGGGGSGGGGSDIQMTQSPSSLSASVGDRVTI |
| | | TCQASQGIRKYLNWYQQKPGKVPKLLIYDASNLETGVPSRF |
| | | SGSGSGTDFTFAISSLQPEDTATYYCQQYDDFPFTFGQGTRLE |
| | | IKRPGTPLGDTTHTSGYPYDVPDYALEGSGTTSGTTRLLSGH |
| | | TCFTLTGLLGTLVTMGLLT |
| 147. | GPI-C1-14 | DIQMTQSPSSLSASVGDRVTITCQASQGIRKYLNWYQQKPG |
| | | KVPKLLIYDASNLETGVPSRFSGSGSGTDFTFAISSLQPEDTA |
| | | TYYCQQYDDFPFTFGQGTRLEIKRGGGGSGGGGSGGGGSQV |
| | | QLVESGGGVVQPGRSLRLSCAASGFTLSGYGMHWVRQAPG |
| | | KGLEWVSLISYDGSNKYYADSVKGRFTISRDDSKNTLYLRM |
| | | NSLRAEDTAVYYCARGRNDFWSGYYTAGMDVWGQGTTVT |
| | | VSSPGTPLGDTTHTSGYPYDVPDYALEGSGTTSGTTRLLSGH |
| | | TCFTLTGLLGTLVTMGLLT |
| 148. | CD8a signal | MALPVTALLLPLALLLHAARP |
| | peptide | |
| 149. | GPI | GSGTTSGTTRLLSGHTCFTLTGLLGTLVTMGLLT |
| | attachment | |
| | sequence | |
| 150. | Linker | PGTPLGDTTHTSG |
| | (derived from | |
| | human IgG3 | |
| | hinge region) | |
| 151. | HA tag | YPYDVPDYALE |
| 152. | Linker + HA | PGTPLGDTTHTSGYPYDVPDYALE |
| | tag | |
| 153. | GFP | GSGATNFSLLKQAGDVEENPGPMVSKGEELFTGVVPILVELD |
| | | GDVNGHKFSVSGEGEGDATYGKLTLKFICTTGKLPVPWPTL |
| | | VTTLTYGVQCFSRYPDHMKQHDFFKSAMPEGYVQERTIFFK |
| | | DDGNYKTRAEVKFEGDTLVNRIELKGIDFKEDGNILGHKLE |
| | | YNYNSHNVYIMADKQKNGIKVNFKIRHNIEDGSVQLADHYQ |
| | | QNTPIGDGPVLLPDNHYLSTQSALSKDPNEKRDHMVLLEFV |
| | | TAAGITLGMDELYKSG |
| 154 | X5 HC-CDR1 | MYGFN |
| 155. | X5 HC-CDR2 | GIIPIFGTSNYAQKFRG |
| 156. | X5 HC-CDR3 | DFGPDWEDGDSYDGSGRGFFDF |
| 157. | X5 LC-CDR1 | RASQSVSSGSLA |
| 158. | X5 LC-CDR2 | GASTRAT |
| 159. | X5 LC-CDR3 | QQYGTSPYT |
| 160. | X5 VH | RVQLLEQSGAEVKKPGSSVQVSCKASGGTFSMYGFNWVRQAPGH |
| | | GLEWMGGIIPIFGTSNYAQKFRGRVTFTADQATSTAYMELTNLRS |
| | | DDTAVYYCARDFGPDWEDGDSYDGSGRGFFDFWGQGTLVTVSS |
| 161. | X5 VL | VLTQSPGTLSLSAGERATLSCRASQSVSSGSLAWYQQKPGQAPRL |
| | | LIYGASTRATGIPDRFSGSGSGTDFTLTIGRLEPEDLAVYYCQQYGT |
| | | SPYTFGQGTKLEIKR |
| 162. | X5 scFv | RVQLLEQSGAEVKKPGSSVQVSCKASGGTFSMYGFNWVRQAPGH |
| | | GLEWMGGIIPIFGTSNYAQKFRGRVTFTADQATSTAYMELTNLRS |
| | | DDTAVYYCARDFGPDWEDGDSYDGSGRGFFDFWGQGTLVTVSS |
| | | AGGGGSGGGGSGGGGSVLTQSPGTLSLSAGERATLSCRASQSVSS |
| | | GSLAWYQQKPGQAPRLLIYGASTRATGIPDRFSGSGSGTDFTLTIG |
| | | RLEPEDLAVYYCQQYGTSPYTFGQGTKLEIKR |
| 163. | GPI-X5 | TMKHLWFFLLLVAAPRWVLSSRVQLLEQSGAEVKKPGSSVQVSC |
| | | KASGGTFSMYGFNWVRQAPGHGLEWMGGIIPIFGTSNYAQKFRG |
| | | RVTFTADQATSTAYMELTNLRSDDTAVYYCARDFGPDWEDGDSY |
| | | DGSGRGFFDFWGQGTLVTVSSAGGGGSGGGGSGGGGSVLTQSPG |
| | | TLSLSAGERATLSCRASQSVSSGSLAWYQQKPGQAPRLLIYGASTR |
| | | ATGIPDRFSGSGSGTDFTLTIGRLEPEDLAVYYCQQYGTSPYTFGQ |
| | | GTKLEIKRPGTPLGDTTHTSGYPYDVPDYALEGSGTTSGTTRLLSG |
| | | HTCFTLTGLLGTLVTMGLLT |
| 164. | signal peptide | TMKHLWFFLLLVAAPRWVLSS |
| | of human | |
| | antibody heavy | |
| | chain | |
| 165. | Consensus | CC(A/T)6GG |
| | sequence in | |
| | Erg promoter | |
| 166. | CD8 | TTTPAPRPPTPAPTIASQPLSLRPEACRPAAGGAVHTRGLDFA |
| | transmembrane | CDIYIWAPLAGTCGVLLLSLVITLYC |
| | domain | |
| 167. | CAR-C2-11 | MALPVTALLLPLALLLHAARPQVQLQQSGPEVVKPGASVK |
| | | MSCKASGYTFTSYVIHWVRQKPGQGLDWIGYINPYNDGTD |
| | | YDEKFKGKATLTSDTSTSTAYMELSSLRSEDTAVYYCAREK |
| | | DNYATGAWFAYWGQGTLVTVSSAGGGGSGGGGSGGGGSD |
| | | IVMTQSPDSLAVSLGERVTMNCKSSQSLLYSTNQKNYLAWY |
| | | QQKPGQSPKLLIYWASTRESGVPDRFSGSGSGTDFTLTISSVQ |
| | | AEDVAVYYCQQYYSYRTFGGGTKLEIKRTTTPAPRPPTPAPT |
| | | IASQPLSLRPEACRPAAGGAVHTRGLDFACDIYIWAPLAGTC |
| | | GVLLLSLVITLYC |
| 168. | CAR-C1-13 | MALPVTALLLPLALLLHAARPQVQLVESGGGVVQPGRSLRL |
| | | SCAASGFTLSGYGMHWVRQAPGKGLEWVSLISYDGSNKYY |
| | | ADSVKGRFTISRDDSKNTLYLRMNSLRAEDTAVYYCARGRN |
| | | DFWSGYYTAGMDVWGQGTTVTVSSGGGGSGGGGSGGGGS |
| | | DIQMTQSPSSLSASVGDRVTITCQASQGIRKYLNWYQQKPG |
| | | KVPKLLIYDASNLETGVPSRFSGSGSGTDFTFAISSLQPEDTA |
| | | TYYCQQYDDFPFTFGQGTRLEIKRTTTPAPRPPTPAPTIASQP |
| | | LSLRPEACRPAAGGAVHTRGLDFACDIYIWAPLAGTCGVLL |
| | | LSLVITLYC |
| 169. | CAR-C2-13 | MALPVTALLLPLALLLHAARPQVQLQQWGAGLLKPSETLSL |
| | | TCAVYGGSFSGYYWSWIRQPPGKGLEWIGEINHSGSTNYNP |
| | | SLKSRVTISVDTSKNQFSLKLSSVTAADTAVYYCARVINWFD |
| | | PWGQGTLVTGGGGSGGGGSGGGGSDIQMTQSPSSVSASVG |
| | | DRVTITCRASQDISSWLAWYQHKPGKAPKLLIYAASSLQSGV |
| | | PSRFSGSGSGTDFTLTISSLQPEDFATYYCQQANSFPYTFGQG |
| | | TKLEIKTTTPAPRPPTPAPTIASQPLSLRPEACRPAAGGAVHT |
| | | RGLDFACDIYIWAPLAGTCGVLLLSLVITLYC |
| 170. | CAR-10E8 | MALPVTALLLPLALLLHAARPEVQLVESGGGLVKPGGSLRL |
| | | SCSASGFDFDNAWMTWVRQPPGKGLEWVGRITGPGEGWSV |
| | | DYAAPVEGRFTISRLNSINFLYLEMNNLRMEDSGLYFCARTG |
| | | KYYDFWSGYPPGEEYFQDWGRGTLVTVSSGGGGSGGGGSG |
| | | GGGSSYELTQETGVSVALGRTVTITCRGDSLRSHYASWYQK |
| | | KPGQAPILLFYGKNNRPSGVPDRFSGASGNRASLTISGAQAE |
| | | DDAEYYCSSRDKSGSRLSVFGGGTKLTVLTTTPAPRPPTPAP |
| | | TIASQPLSLRPEACRPAAGGAVHTRGLDFACDIYIWAPLAGT |
| | | CGVLLLSLVITLYC |
| 171. | CAR-C34 | MALPVTALLLPLALLLHAARPTMKHLWFFLLLVAAPRWVLS |
| | | SWMEWDREINNYTSLIHSLIEESQNQQEKNEQELLTTTPAPR |
| | | PPTPAPTIASQPLSLRPEACRPAAGGAVHTRGLDFACDIYIWA |
| | | PLAGTCGVLLLSLVITLYC |

1. An engineered cell comprising a nucleic acid encoding a surface molecule comprising:
a) a binding moiety, wherein the binding moiety
i) specifically binds to a T cell surface antigen and prevents the binding of the T cell surface antigen to its cognitive ligand on HIV, or
ii) specifically binds to a HIV antigen competitively with an anti-HIV antibody, or binds to the same epitope as that of the anti-HIV antibody and prevents HIV from infecting the engineered cell; and
b) a membrane domain that tethers the molecule to the membrane or facilitates the tethering of the surface molecule to the membrane; wherein the membrane domain is derived from, or is a transmembrane domain from CD4, CD8, CD28, 4-1 BB, or PD-1, or the membrane domain comprises a Glycosylphosphatidylinositol(GPI) attachment signal sequence.

2. (canceled)

3. The engineered cell of claim 1, wherein the T cell surface antigen is selected from the group consisting of CCR5, CD4, and CXCR4.

4-6. (canceled)

7. The engineered cell of claim 3, wherein the binding moiety specifically binds to CCR5 competitively with C1-13, C1-14, C1-11, C1-12, C1-814, or C1-816, or specifically binds to the same epitope as that of C1-13, C1-14, C1-11, C1-12, C1-814, or C1-816.

8. The engineered cell of claim 7, wherein the binding moiety comprises an antibody moiety comprising a heavy chain variable region (V_{H}) and a light chain variable region (V_{L}), wherein:
(1) the V_{H} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14,
(2) V_{H} comprises the HC-CDR1 comprising the amino acid sequence of SEQ ID NO: 125, the HC-CDR2 comprising the amino acid sequence of SEQ ID NO: 126, and the HC-CDR3 comprising the amino acid sequence of SEQ ID NO: 127, and the V_{L} comprises the LC-CDR1 comprising the amino acid sequence of SEQ ID NO: 128, the LC-CDR2 comprising the amino acid sequence of SEQ ID NO: 129, and the LC-CDR3 comprising the amino acid sequence of SEQ ID NO: 130, or
(3) the V_{H} comprises the HC-CDR1 comprising the amino acid sequence of SEQ ID NO: 15, the HC-CDR2 comprising the amino acid sequence of SEQ ID NO: 16, and the HC-CDR3 comprising the amino acid sequence of SEQ ID NO: 17, and the V_{L} comprises the LC-CDR1 comprising the amino acid sequence of SEQ ID NO: 18, the LC-CDR2 comprising the amino acid sequence of SEQ ID NO: 19, and the LC-CDR3 comprising the amino acid sequence of SEQ ID NO: 20.

9. The engineered cell of claim 8, wherein:
(1) the V_{H} comprising the amino acid sequence set forth in SEQ ID NO: 75 or a variant comprising an amino acid sequence having at least about 80% sequence identity, and the V_{L} comprising the amino acid sequence of SEQ ID NO: 76 or a variant comprising an amino acid sequence having at least about 80% sequence identity,
(2) the V_{H} comprising the amino acid sequence set forth in SEQ ID NO: 131 or a variant comprising an amino acid sequence having at least about 80% sequence identity, and the V_{L} comprising the amino acid sequence of SEQ ID NO: 132 or a variant comprising an amino acid sequence having at least about 80% sequence identity, or
(3) the V_{H} comprising the amino acid sequence set forth in SEQ ID NO: 77 or 79, or a variant comprising an amino acid sequence having at least about 80% sequence identity, and the V_{L} comprising the amino acid sequence of SEQ ID NO: 78 or 80, or a variant comprising an amino acid sequence having at least about 80% sequence identity.

10. (canceled)

11. The engineered cell of claim 3, wherein the T cell surface antigen is CD4, wherein the binding moiety specifically binds to CD4 competitively with C2-05, C2-11, or C2-13, or specifically binds to the same epitope as that of C2-05, C2-11, or C2-13.

12. (canceled)

13. The engineered cell of claim 11, wherein the binding moiety comprises an antibody moiety comprising a heavy chain variable region (V_{H}) and a light chain variable region (V_{L}), wherein:
(1) the V_{H} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38,
(2) the V_{H} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26, or
(3) the V_{H} comprises the HC-CDR1 comprising the amino acid sequence of SEQ ID NO: 27, the HC-CDR2 comprising the amino acid sequence of SEQ ID NO: 28, and the HC-CDR3 comprising the amino acid sequence of SEQ ID NO: 29, and the V_{L} comprises the LC-CDR1 comprising the amino acid sequence of SEQ ID NO: 30, the LC-CDR2 comprising the amino acid sequence of SEQ ID NO: 31, and the LC-CDR3 comprising the amino acid sequence of SEQ ID NO: 32.

14. The engineered cell of claim 13, wherein:
(1) the V_{H} comprising the amino acid sequence set forth in SEQ ID NO: 85, or a variant comprising an amino acid sequence having at least about 80% sequence identity, and the V_{L} comprising the amino acid sequence of SEQ ID NO: 86, or a variant comprising an amino acid sequence having at least about 80% sequence identity,
(2) the V_{H} comprising the amino acid sequence set forth in SEQ ID NO: 81, or a variant comprising an amino acid sequence having at least about 80% sequence identity, and the V_{L} comprising the amino acid sequence of SEQ ID NO: 82, or a variant comprising an amino acid sequence having at least about 80% sequence identity, or
(3) the V_{H} comprising the amino acid sequence set forth in SEQ ID NO: 83, or a variant comprising an amino acid sequence having at least about 80% sequence identity, and the V_{L} comprising the amino acid sequence of SEQ ID NO: 84, or a variant comprising an amino acid sequence having at least about 80% sequence identity.

15. (canceled)

16. The engineered cell of claim 1, wherein the surface molecule comprises a binding moiety that specifically binds to HIV competitively with 10-1074, 10E8, or PGT121, or specifically binds to the same epitope as that of 10-1074, 10E8, or PGT121.

17-19. (canceled)

20. The engineered cell of claim 16, wherein the surface molecule comprises a binding moiety that specifically binds to HIV competitively with 10E8, or specifically binds to the same epitope as that of 10E8; wherein the binding moiety comprises an antibody moiety comprising a heavy chain variable region (V_{H}) and a light chain variable region (VU, wherein the V_{H} comprises the HC-CDR1 comprising the amino acid sequence of SEQ ID NO: 63, the HC-CDR2 comprising the amino acid sequence of SEQ ID NO: 64, and the HC-CDR3 comprising the amino acid sequence of SEQ ID NO: 65, and the V_{L} comprises the LC-CDR1 comprising the amino acid sequence of SEQ ID NO: 66, the LC-CDR2 comprising the amino acid sequence of SEQ ID NO: 67, and the LC-CDR3 comprising the amino acid sequence of SEQ ID NO: 68.

21. (canceled)

22. The engineered cell of claim 20, wherein the antibody moiety comprises a) the V_{H} comprising the amino acid sequence set forth in SEQ ID NO: 89, or a variant comprising an amino acid sequence having at least about 80% sequence identity, and b) the V_{L} comprising the amino acid sequence of SEQ ID NO: 90, or a variant comprising an amino acid sequence having at least about 80% sequence identity.

23. The engineered cell of claim 16, wherein the surface molecule comprises a binding moiety that specifically binds to HIV competitively with PGT121, or specifically binds to the same epitope as that of PGT121; wherein the binding moiety comprises an antibody moiety comprising a heavy chain variable region (V_{H}) and a light chain variable region (VU, wherein the V_{H} comprises the HC-CDR1 comprising the amino acid sequence of SEQ ID NO: 69, the HC-CDR2 comprising the amino acid sequence of SEQ ID NO: 70, and the HC-CDR3 comprising the amino acid sequence of SEQ ID NO: 71, and the V_{L} comprises the LC-CDR1 comprising the amino acid sequence of SEQ ID NO: 72, the LC-CDR2 comprising the amino acid sequence of SEQ ID NO: 73, and the LC-CDR3 comprising the amino acid sequence of SEQ ID NO: 74.

24. (canceled)

25. The engineered cell of claim 23, wherein the antibody moiety comprises a) the V_{H} comprising the amino acid sequence set forth in SEQ ID NO: 91, or a variant comprising an amino acid sequence having at least about 80% sequence identity, and b) the V_{L} comprising the amino acid sequence of SEQ ID NO: 92, or a variant comprising an amino acid sequence having at least about 80% sequence identity.

26-29. (canceled)

30. The engineered cell of claim 1, wherein the binding moiety is a sdAb, a scFv, a Fab′, a (Fab′)₂, an Fv, or a peptide ligand.

31. (canceled)

32. The engineered cell of claim 1, wherein the engineered cell is a stem cell, an immune cell or a Natural Killer cell.

33-36. (canceled)

37. The engineered cell of claim 1, wherein the GPI attachment signal sequence comprises an amino acid sequence set forth in SEQ ID NO: 149.

38-53. (canceled)

54. A surface molecule comprising:
a) a binding moiety that specifically binds to a T cell surface antigen and prevents the binding of the T cell surface antigen to its cognitive ligand on HIV, a binding moiety that specifically binds to a HIV antigen competitively with an anti-HIV antibody, or binds to the same epitope as that of the anti-HIV antibody and prevents HIV from infecting the engineered cell, or an inhibitory moiety that inhibits membrane fusion of HIV; and
b) a membrane domain that can tether the molecule to a cell membrane or facilitate the tethering of the surface molecule to the cell membrane after being expressed in a cell,
wherein upon being expressed by a cell, the cell confers herd immunity against HIV; wherein the cell expresses CCR5, CD4 or CXCR4.

55-56. (canceled)

57. An engineered cell expressing the surface molecule of claim 54, wherein the cell is a stem cell or an immune cell.

58-59. (canceled)

60. A pharmaceutical composition comprising the engineered cell claim 1.

61-64. (canceled)

65. A method of treating an individual infected with HIV, comprising administering to the individual an effective amount of the engineered cell of claim 1.

66-69. (canceled)
